(19) United States
(12) Patent Application Publication
(10) Pub. No.: US 2025/0101525 A1
(43) Pub. Date: Mar. 27, 2025

(54) BIOMARKERS AND USES THEREFOR

(71) Applicant: Griffith University, Nathan (AU)

(72) Inventors: Chamindie PUNYADEERA, Nathan (AU); Chameera WEERAMANGE, Nathan (AU)

(21) Appl. No.: 18/730,665

(22) PCT Filed: Jan. 23, 2023
(86) PCT No.: PCT/AU2023/050039

§ 371 Date: Jul. 19, 2024

(30) Foreign Application Priority Data
Jan. 21, 2022 (AU) 2022900109

(51) Int. Cl. C12Q 1/6886; C12Q 1/48

(52) U.S. Cl. CPC C12Q 1/6886; C12Q 1/485; C12Q 2600/112; C12Q 2600/118; C12Q 2600/158; C12Q 2600/178

(57) ABSTRACT

Disclosed herein are biomarkers of cancer. More particularly, the present disclosure relates to miRNA biomarkers and their use in methods, compositions, apparatuses, devices and kits for determining an indicator that is useful for assessing a likelihood that oropharyngeal cancer is present or absent in a human subject, or for assessing a likelihood of a human subject with OPC having a poor survival prognosis or good survival prognosis.

### RELATED APPLICATIONS

This application claims priority to Australian Provisional Application No. 2022900109 entitled “Biomarkers and uses therefor” filed 21 Jan. 2022, the contents of which are incorporated herein by reference in their entirety.

### FIELD

This disclosure relates generally to biomarkers of cancer. More particularly, the present disclosure relates to miRNA biomarkers and their use in methods, compositions, apparatuses, devices and kits for determining an indicator that is useful for assessing a likelihood that oropharyngeal cancer is present or absent in a human subject, or for assessing a likelihood of a human subject with OPC having a poor survival prognosis or good survival prognosis.

### BACKGROUND

Micro RNA (miRNA) mediated messenger RNA regulation can be identified as one of the most efficient post-transcriptional gene expression regulation modalities that cells employ. As such, these approximately 22-nucleotide long, small non-coding RNAs play an integral role in virtually every cellular process including development, differentiation, and maintenance of cells (Wilczynska et al., Cell Death & Differentiation. 2015; 22(1):22-33). Due to their dynamic engagement in biological pathways, cells maintain a precise composition of miRNAs required depending on the cellular state (Treiber et al., Nature Reviews Molecular Cell Biology. 2019; 20(1):5-20; O'Brien et al., Mechanisms of Actions, and Circulation. 2018; 9(402)). However, in disease conditions, this composure often gets disturbed, leading to active or passive changes in miRNA expression. Hence, miRNAs are recognized as robust diagnostic, prognostics, monitoring, and therapeutic targets for a range of disease conditions (Rupaimoole et al., Nature Reviews Drug Discovery. 2017; 16(3):203-22; Condrat et al., Cells. 2020; 9(2):276; Naidu et al., Journal of Hematology & Oncology. 2015; 8(1)).

Cancer is one such disease where dysregulation of miRNA is well documented (Chakraborty et al., Tumor Biology. 2016; 37(10):13039-48; Peng et al., Signal Transduction and Targeted Therapy. 2016; 1(1):15004). These changes are not only detectable in tumor tissue and tumor microenvironment, but also can be detected in surrounding body fluids (Lu et al., Journal of Allergy and Clinical Immunology. 2018; 141(4):1202-7; Rupaimoole et al., Cancer Discovery. 2016; 6(3):235-46). As such, liquid biopsy-based miRNA evaluation can be considered as a potential tool for cancer detection. Oropharyngeal cancer (OPC), which is a subject of the present disclosure, is no exception for miRNA expression changes (Gao et al., Cancer. 2013; 119(1):72-80; Miller et al., Am J Pathol. 2015; 185(3):679-92; Salazar et al., Expert Review of Molecular Diagnostics. 2014; 14(8):1033-40). However, liquid biopsy-based miRNA changes that enable OPC identification are yet to be elucidated. Being in close contact with these tumors, saliva provides an ideal avenue for such biomarker identification.

The presence of two clinically and biologically distinct categories of OPC depending on the etiology, however, complicates miRNA identification for OPC detection. A subset of OPC is caused by carcinogenic types of Human papillomavirus (HPV) and the others are mainly caused by behavioral risk factors such as smoking and excessive alcohol consumption (Chow et al., N Engl J Med. 2020; 382(1):60-72). Even though behavioral risk factors accounted for the majority of OPCs in the past, the epidemiological and demographic landscape of OPC has been changed considerably over the last two decades due to the alarming rise in HPV associated OPC incidence, especially in the western world (Gillison et al., J Clin Oncol. 2015; 33(29):3235-42; Anantharaman et al., Int J Cancer. 2017; 140(9):1968-75).

### SUMMARY

The present disclosure arises from the determination that certain miRNA biomarkers from saliva have strong discrimination performance for differentiating between human subjects with HPV-positive OPC and HPV-positive controls, whilst others have strong discrimination performance for distinguishing between human subjects with HPV-negative OPC and HPV-negative controls, and still others can be used to differentiate OPC from controls regardless of their HPV status. Additionally, a single miRNA biomarker (Hsa-miR-07-5p) was found to be a predictor of patient survival. Based on these determinations, methods, apparatuses, compositions, devices and kits are disclosed, which take advantage of biomarkers disclosed herein to determine a likelihood that HPV-positive OPC, OPC-negative HPV infection (i.e., OPC-negative, HPV-positive condition), HPV-negative OPC (i.e., OPC-positive, HPV-negative condition), OPC regardless of HPV status (also referred to herein as HPV-agnostic OPC) or an OPC-negative, HPV-negative condition is present or absent in a human subject, including a human subject presenting with at least one clinical sign of OPC. In certain embodiments, methods, apparatuses, compositions, devices and kits are disclosed for predicting survival of patients with OPC.

Accordingly, in one aspect, disclosed herein are methods for determining an indicator used in assessing a likelihood that HPV-positive OPC or OPC-negative HPV infection is present or absent in a human subject. These methods general comprise, consist or consist essentially of:
(1) determining a biomarker value for at least one miRNA biomarker (e.g., 1, 2 or 3 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-501-3p and Hsa-miR-548K; and
(2) determining the indicator using the biomarker value(s).

Another aspect of the present disclosure provides methods for determining an indicator used in assessing a likelihood that HPV-positive OPC or an OPC-negative, HPV-positive condition is present or absent in a human subject. These methods general comprise, consist or consist essentially of:
(1) determining a biomarker value for at least one miRNA biomarker (e.g., 1, 2, 3, 4, 5, 6, 7, 8 or 9 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K; and
(2) determining the indicator using the biomarker value(s).

Disclosed herein in another aspect are methods for determining an indicator used in assessing a likelihood that HPV-negative OPC or an OPC-negative, HPV-negative condition is present or absent in a human subject. These methods general comprise, consist or consist essentially of:
(1) determining a biomarker value for at least one miRNA biomarker (e.g., 1, 2, 3, 4, 5, 6 or 7 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K; and
(2) determining the indicator using the biomarker value(s).

Disclosed herein in yet another aspect are methods for determining an indicator used in assessing a likelihood that HPV-negative OPC or an OPC-negative, HPV-negative condition is present or absent in a human subject. These methods general comprise, consist or consist essentially of:
(1) determining a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-449a, Hsa-miR-07-5p, Hsa-miR-501-3p, Hsa-miR-1290 and Hsa-miR-548K; and
(2) determining the indicator using the biomarker value(s).

In yet another aspect, methods are disclosed for determining an indicator used in assessing a likelihood that HPV-agnostic OPC or an OPC-negative condition is present or absent in a human subject. These methods general comprise, consist or consist essentially of:
(1) determining a biomarker value for at least one miRNA biomarker (e.g., 1, 2, 3 or 4 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-3614-5p, Hsa-miR-501-3p, Hsa-miR-1290 and Hsa-miR-1246; and
(2) determining the indicator using the biomarker value(s).

A further aspect of the present disclosure provides methods for determining an indicator used in assessing a likelihood that HPV-agnostic OPC or an OPC-negative condition (e.g., HPV-positive, OPC-negative condition, HPV-negative, OPC-negative condition) is present or absent in a human subject. These methods general comprise, consist or consist essentially of:
(1) determining a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-449a, Hsa-miR-3614-5p, Hsa-miR-07-5p, Hsa-miR-501-3p, Hsa-miR-1290 and Hsa-miR-548K; and
(2) determining the indicator using the biomarker value(s).

In any of the aspects disclosed herein, the subject suitably has at least one clinical sign of OPC (e.g., sore throat that does not go away, a lump in the throat, mouth or neck, trouble swallowing, trouble opening the mouth fully, trouble moving the tongue, weight loss for no reason, ear pain, a change in voice, a white patch on the tongue or lining of the mouth that does not go away, and coughing up blood). Alternatively, the subject may be asymptomatic but at risk of OPC, illustrative examples of which include subjects having a history of smoking or oral tobacco use, drinking mate (i.e., a stimulant drink common in South America), chewing betel quid (i.e., a stimulant commonly used in parts of Asia), are HPV-positive, are HIV-positive, have had multiple sexual partners and/or engage in oral sex. In other embodiments, the subject may be a healthy subject.

In some embodiments, the methods further comprise applying a function to biomarker values to yield at least one functionalized biomarker value and determining the indicator using the at least one functionalized biomarker value. In representative examples, the function includes at least one of: (a) multiplying biomarker values; (b) dividing biomarker values; (c) adding biomarker values; (d) subtracting biomarker values; (e) a weighted sum of biomarker values; (f) a log sum of biomarker values; (g) a geometric mean of biomarker values; (h) a sigmoidal function of biomarker values; and (i) normalization of biomarker values.

In some embodiments, the methods further comprise combining the biomarker values to provide a composite score and determining the indicator using the composite score. In non-limiting examples of this type, the biomarker values are combined by adding, multiplying, subtracting, and/or dividing biomarker values.

In some embodiments, the methods further comprise analyzing the biomarker value(s) or composite score with reference to one or more reference biomarker values, value ranges or cut-off values, or reference composite scores, composite score ranges or composite score cut-offs, to determine the indicator. A respective reference biomarker value, value range or cut-off value, or reference composite score, composite score range or composite score cut-off may be a biomarker value, value range or cut-off value, or reference composite score, composite score range or composite score cut-off corresponding to a control subject or control population of subjects. The condition of the control subject or control population of subjects is suitably the condition analyzed by the indicator. In other embodiments, the condition of the control subject or control population of subjects is a different condition than the condition analyzed by the indicator. In representative examples of this type, the condition analyzed by the indicator represents a first condition assessed by a method disclosed herein, and the condition of the control subject or control population of subjects represents a second condition assessed by that method.

Suitably, the indicator indicates a likelihood of a presence of a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition, if the biomarker value(s) or composite score is indicative of the level of the biomarker(s) in the sample that correlates with an increased likelihood of a presence of the condition relative to a predetermined reference biomarker value, value range or cut-off value, or to a predetermined reference composite score, composite score range or composite score cut-off.

Alternatively, the indicator may indicate a likelihood of an absence of a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition, if the biomarker value(s) or composite score is indicative of the level of the biomarker(s) in the sample that correlates with an increased likelihood of an absence of the condition relative to a predetermined reference biomarker value, value range or cut-off value, or to a predetermined reference composite score, composite score range or composite score cut-off.

In any of the aspects disclosed herein, individual biomarker values may represent a measured amount or concentration of a corresponding miRNA biomarker in the sample. Alternatively, individual biomarker values may be a logarithmic representation of a measured amount or concentration of a corresponding miRNA biomarker in the sample.

Another aspect of the present disclosure provides methods for determining an indicator used in assessing a likelihood of a human subject with OPC having a survival prognosis selected from a decreased or poor survival prognosis and an increased or good survival prognosis. These methods generally comprise, consist or consist essentially of:
(1) determining a biomarker value for Hsa-miR-07-5p in a saliva sample obtained from the subject, wherein the biomarker value is indicative of a level of Hsa-miR-07-5p in the sample; and
(2) determining the indicator using the biomarker value.

These methods may further comprise analyzing the biomarker value with reference to a corresponding reference biomarker value, value range or cut-off value, to determine the indicator. In representative examples of this type, the indicator indicates a likelihood of a decreased or poor survival prognosis if the biomarker value for Hsa-miR-07-5p is indicative of a level of Hsa-miR-07-5p in the sample that correlates with an increased likelihood of a decreased or poor survival prognosis relative to a predetermined reference biomarker value, value range or cut-off value. Alternatively, the indicator may indicate a likelihood of an increased or good survival prognosis if the biomarker value for Hsa-miR-07-5p is indicative of a level of Hsa-miR-07-5p in the sample that correlates with an increased likelihood of an increased or good survival prognosis relative to a predetermined reference biomarker value, value range or cut-off value.

Disclosed herein in another aspect are apparatuses for determining an indicator used in assessing a likelihood that HPV-positive OPC or OPC-negative HPV infection is present or absent in a human subject. These apparatuses general comprise, consist or consist essentially of at least one electronic processing device that:
determines a biomarker value for at least one miRNA biomarker (e.g., 1, 2 or 3 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-501-3p and Hsa-miR-548K; and
determines the indicator using the biomarker value(s).

In a further aspect, apparatuses are disclosed for determining an indicator used in assessing a likelihood that HPV-positive OPC or an OPC-negative, HPV-positive condition is present or absent in a human subject. These apparatuses general comprise, consist or consist essentially of at least one electronic processing device that:
determines a biomarker value for at least one miRNA biomarker (e.g., 1, 2, 3, 4, 5, 6, 7, 8 or 9 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K; and
determines the indicator using the biomarker value(s).

In still another aspect, apparatuses are disclosed for determining an indicator used in assessing a likelihood that HPV-negative OPC or an OPC-negative, HPV-negative condition is present or absent in a human subject. These apparatuses general comprise, consist or consist essentially of at least one electronic processing device that:
determines a biomarker value for at least one miRNA biomarker (e.g., 1, 2, 3, 4, 5, 6 or 7 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K; and
determines the indicator using the biomarker value(s).

Disclosed herein in yet another aspect are apparatuses for determining an indicator used in assessing a likelihood that HPV-negative OPC or an OPC-negative, HPV-negative condition is present or absent in a human subject. These apparatuses general comprise, consist or consist essentially of at least one electronic processing device that:
determines a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-449a, Hsa-miR-07-5p, Hsa-miR-501-3p, Hsa-miR-1290 and Hsa-miR-548K; and
determines the indicator using the biomarker value(s).

Another aspect of the present disclosure provides apparatuses for determining an indicator used in assessing a likelihood that HPV-agnostic OPC or an OPC-negative condition is present or absent in a human subject. These apparatuses general comprise, consist or consist essentially of at least one electronic processing device that:
determines a biomarker value for at least one miRNA biomarker (e.g., 1, 2, 3 or 4 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-3614-5p, Hsa-miR-501-3p, Hsa-miR-1290 and Hsa-miR-1246; and
determines the indicator using the biomarker value(s).

A further aspect of the present disclosure provides apparatuses for determining an indicator used in assessing a likelihood that HPV-agnostic OPC or an OPC-negative condition (e.g., HPV-positive, OPC-negative condition, HPV-negative, OPC-negative condition) is present or absent in a human subject. These apparatuses general comprise, consist or consist essentially of at least one electronic processing device that:
determines a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-449a, Hsa-miR-3614-5p, Hsa-miR-07-5p, Hsa-miR-501-3p, Hsa-miR-1290 and Hsa-miR-548K; and
determines the indicator using the biomarker value(s).

In still another aspect, apparatuses are disclosed for determining an indicator used in assessing a likelihood of a human subject with OPC having a survival prognosis selected from a decreased or poor survival prognosis and an increased or good survival prognosis. These apparatuses general comprise, consist or consist essentially of at least one electronic processing device that:
determines a biomarker value for Hsa-miR-07-5p in a saliva sample obtained from the subject, wherein the biomarker value is indicative of a level of Hsa-miR-07-5p in the sample; and
determines the indicator using the biomarker value.

In yet another aspect, disclosed herein are compositions, suitably for determining an indicator used in assessing a likelihood that HPV-positive OPC or OPC-negative HPV infection is present or absent in a human subject. These compositions generally comprise, consist or consist essentially of a mixture of a DNA polymerase (e.g., a thermostable DNA polymerase), saliva cDNA from a human subject (e.g., a subject with at least one clinical sign of OPC, a subject at risk of OPC, or a healthy subject), wherein the saliva cDNA comprises at least one cDNA biomarker (e.g., 1, 2 or 3 cDNA biomarkers) selected from Hsa-miR-194-5p cDNA, Hsa-miR-501-3p cDNA and Hsa-miR-548K cDNA, and wherein the composition further comprises for a respective cDNA biomarker at least one oligonucleotide primer or probe that hybridizes to the cDNA biomarker.

Another aspect of the present disclosure provides compositions, suitably for determining an indicator used in assessing a likelihood that HPV-positive OPC or an OPC-negative, HPV-positive condition is present or absent in a human subject. These compositions generally comprise, consist or consist essentially of a mixture of a DNA polymerase (e.g., a thermostable DNA polymerase), saliva cDNA from a human subject (e.g., a subject with at least one clinical sign of OPC, a subject at risk of OPC, or a healthy subject), wherein the saliva cDNA comprises at least one cDNA biomarker (e.g., 1, 2, 3, 4, 5, 6, 7, 8 or 9 cDNA biomarkers) selected from Hsa-miR-194-5p cDNA, Hsa-miR-449a cDNA, Hsa-miR-3614-5p cDNA, Hsa-miR-07-5p cDNA, Hsa-miR-3529-3p cDNA, Hsa-miR-99A-3p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

Disclosed herein in yet another aspect are compositions, suitably for determining an indicator used in assessing a likelihood that HPV-negative OPC or an OPC-negative, HPV-negative condition is present or absent in a human subject. These compositions generally comprise, consist or consist essentially of a mixture of a DNA polymerase (e.g., a thermostable DNA polymerase), saliva cDNA from a human subject (e.g., a subject with at least one clinical sign of OPC, a subject at risk of OPC, or a healthy subject), wherein the saliva cDNA comprises at least one cDNA biomarker (e.g., 1, 2, 3, 4, 5, 6 or 7 cDNA biomarkers) selected from Hsa-miR-215-3p cDNA, Hsa-miR-194-5p cDNA, Hsa-miR-449a cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

Disclosed herein in another aspect are compositions, suitably for determining an indicator used in assessing a likelihood that HPV-negative OPC or an OPC-negative, HPV-negative condition is present or absent in a human subject. These compositions generally comprise, consist or consist essentially of a mixture of a DNA polymerase (e.g., a thermostable DNA polymerase), saliva cDNA from a human subject (e.g., a subject with at least one clinical sign of OPC, a subject at risk of OPC, or a healthy subject), wherein the saliva cDNA comprises at least one cDNA biomarker (e.g., 1, 2, 3, 4, 5 or 6 cDNA biomarkers) selected from Hsa-miR-194-5p cDNA, Hsa-miR-449a cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

Still another aspect of the present disclosure provides compositions, suitably for determining an indicator used in assessing a likelihood that HPV-agnostic OPC or an OPC-negative condition is present or absent in a human subject. These compositions generally comprise, consist or consist essentially of a mixture of a DNA polymerase (e.g., a thermostable DNA polymerase), saliva cDNA from a human subject (e.g., a subject with at least one clinical sign of OPC, a subject at risk of OPC, or a healthy subject), wherein the saliva cDNA comprises at least one cDNA biomarker (e.g., 1, 2, 3 or 4 cDNA biomarkers) selected from Hsa-miR-3614-5p cDNA, Hsa-miR-501-3p cDNA, Hsa-miR-1290 cDNA and Hsa-miR-1246 cDNA, and wherein the composition further comprises for a respective cDNA biomarker at least one oligonucleotide primer or probe that hybridizes to the cDNA biomarker.

Yet another aspect of the present disclosure provides compositions, suitably for determining an indicator used in assessing a likelihood that HPV-agnostic OPC or an OPC-negative condition (e.g., HPV-positive, OPC-negative condition, HPV-negative, OPC-negative condition) is present or absent in a human subject. These compositions generally comprise, consist or consist essentially of a mixture of a DNA polymerase (e.g., a thermostable DNA polymerase), saliva cDNA from a human subject (e.g., a subject with at least one clinical sign of OPC, a subject at risk of OPC, or a healthy subject), wherein the saliva cDNA comprises at least one cDNA biomarker (e.g., 1, 2, 3, 4, 5 or 6 cDNA biomarkers) selected from Hsa-miR-449a cDNA, Hsa-miR-3614-5p cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

In another aspect, disclosed herein are compositions, suitably for determining an indicator used in assessing a likelihood of a human subject with OPC having a survival prognosis selected from a decreased or poor survival prognosis and an increased or good survival prognosis. These compositions generally comprise, consist or consist essentially of a mixture of a DNA polymerase (e.g., a thermostable DNA polymerase), saliva cDNA from a human subject with OPC, wherein the saliva cDNA comprises a cDNA biomarker corresponding to Hsa-miR-07-5p, and at least one oligonucleotide primer or probe that hybridizes to the cDNA biomarker.

In some embodiments, the compositions comprise for respective cDNA biomarker two oligonucleotide primers that hybridize to opposite complementary strands of a corresponding cDNA biomarker. In some of the same or other embodiments, the compositions comprise for a respective cDNA biomarker an oligonucleotide probe that hybridizes to a corresponding cDNA biomarker or a polynucleotide corresponding to the cDNA biomarker (e.g., a polynucleotide product resulting from nucleic acid amplification of the cDNA biomarker). The oligonucleotide probe may comprise a heterologous label (e.g., a fluorescent label). In embodiments in which the oligonucleotide probe comprises a heterologous label, the labeled oligonucleotide probe may comprise a fluorophore. In representative examples of this type, the labeled oligonucleotide probe further comprises a quencher. In certain embodiments, different labeled oligonucleotide probes are included in the composition for hybridizing to different cDNAs, wherein individual oligonucleotide probes comprise detectably distinct labels (e.g. different fluorophores), or at least a subset of oligonucleotide probes comprises the same label (e.g. same fluorophore). In some embodiments, the compositions comprise for each of at least 1, 2, 3, 4, 5, 6, 7, 8 or 9 cDNA biomarkers at least one oligonucleotide primer and/or probe that hybridizes to the cDNA biomarker. In other embodiments, the compositions comprise for each of up to 1, 2, 3, 4, 5, 6, 7, 8 or 9 cDNA biomarkers at least one oligonucleotide primer and/or probe that hybridizes to the cDNA biomarker. Individual cDNA biomarkers and their corresponding oligonucleotide primer(s) and/or probe(s) may be present in separate reaction vessels or in the same reaction vessel.

In still another aspect, devices are disclosed for nucleic acid amplification of saliva cDNA. These devices comprise a plurality of reaction vessels, wherein individual reaction vessels comprise a composition as broadly described above and elsewhere herein. Devices disclosed herein may consist of 1 to 12, 1 to 10, 1 to 8, 1 to 6 or 1 to 4 reaction vessels (and all integer vessels in between). In non-limiting examples, the devices consist of 1, 2, 3, 4, 5, 6, 7, 8 or 9 reaction vessels. In representative examples of this type, one or more reaction vessels are used for single-plex amplification of cDNA, and/or one or more reaction vessels are used for multiplex amplification of cDNA (e.g., 2-plex, 3-plex, 4-plex, 5-plex or 6-plex amplifications).

In a further aspect, methods are disclosed for inhibiting the development or progression OPC in a human subject. These methods generally comprise, consist or consist essentially of exposing the subject to a treatment regimen for OPC at least in part on the basis that the subject is determined by the indicator-determining method as broadly described above and elsewhere herein as having a likelihood of a presence of an OPC condition selected from HPV-positive OPC, HPV-negative OPC and HPV-agnostic OPC.

In some embodiments, the methods further comprise: taking a sample from the subject and determining an indicator indicative of a likelihood of a presence of the OPC condition using the indicator-determining method. In some of the same or other embodiments, the methods further comprise: sending a sample obtained from the subject to a laboratory at which the indicator is determined according to the indicator-determining method, and optionally receiving the indicator from the laboratory.

Disclosed herein in still another aspect are kits for determining an indicator used in assessing a likelihood that HPV-positive OPC or OPC-negative HPV infection is present or absent in a human subject. These kits generally comprise for each of at least one miRNA biomarker (e.g., 1, 2 or 3 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-194-5p, Hsa-miR-501-3p and Hsa-miR-548K.

In still another aspect, kits are disclosed herein for determining an indicator used in assessing a likelihood that HPV-positive OPC or an OPC-negative, HPV-positive condition is present or absent in a human subject. These kits generally comprise for each of at least one miRNA biomarker (e.g., 1, 2, 3, 4, 5, 6, 7, 8 or 9 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K.

In one aspect, kits are disclosed herein for determining an indicator used in assessing a likelihood that HPV-negative OPC or an OPC-negative, HPV-negative condition is present or absent in a human subject. These kits generally comprise for each of at least one miRNA biomarker (e.g., 1, 2, 3, 4, 5, 6 or 7 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K.

Disclosed herein in yet another aspect are kits for determining an indicator used in assessing a likelihood that HPV-negative OPC or an OPC-negative, HPV-negative condition is present or absent in a human subject. These kits generally comprise for each of at least one miRNA biomarker (e.g., 1, 2, 3, 4, 5 or 6 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-194-5p, Hsa-miR-449a, Hsa-miR-07-5p, Hsa-miR-501-3p, Hsa-miR-1290 and Hsa-miR-548K.

A further aspect of the present disclosure provides kits for determining an indicator used in assessing a likelihood that HPV-agnostic OPC or an OPC-negative condition is present or absent in a human subject. These kits generally comprise for each of at least one miRNA biomarker (e.g., 1, 2, 3 or 4 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-3614-5p, Hsa-miR-501-3p, Hsa-miR-1290 and Hsa-miR-1246.

Yet another aspect of the present disclosure provides kits for determining an indicator used in assessing a likelihood that HPV-agnostic OPC or an OPC-negative condition (e.g., HPV-positive, OPC-negative condition, HPV-negative, OPC-negative condition) is present or absent in a human subject. These kits generally comprise for each of at least one miRNA biomarker (e.g., 1, 2, 3, 4, 5 or 6 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-449a, Hsa-miR-3614-5p, Hsa-miR-07-5p, Hsa-miR-501-3p, Hsa-miR-1290 and Hsa-miR-548K.

In another aspect, kits are disclosed herein for determining an indicator used in assessing a likelihood of a human subject with OPC having a survival prognosis selected from a decreased or poor survival prognosis and an increased or good survival prognosis. These kits generally at least one oligonucleotide primer and/or at least one oligonucleotide probe that hybridizes to Hsa-miR-07-5p or to a cDNA corresponding to Hsa-miR-07-5p.

The kits may further comprise any one or more of: a DNA polymerase (e.g., a thermostable DNA polymerase); for each cDNA corresponding to a respective miRNA biomarker, a pair of forward and reverse oligonucleotide primers that permit nucleic acid amplification of at least a portion of the cDNA to produce an amplicon; for each cDNA corresponding to a respective miRNA biomarker, an oligonucleotide probe that comprises a heterologous label and hybridizes to the cDNA or to an amplicon of the cDNA; one or more reagents for preparing miRNA from a cell or cell population from a saliva sample; one or more reagents for preparing cDNA from the miRNA; one or more reagents for amplifying cDNA; and one or more of deoxynucleotides, buffer(s), positive and negative controls, and reaction vessel(s). Suitably, the kits may further comprise instructions for performing the indicator-determining method as broadly described above and elsewhere herein.

### BRIEF DESCRIPTION OF THE DRAWINGS

FIG. 1 is a graphical representation showing differentially expressed salivary miRNAs. In particular, a volcano plot is presented, showing differentially expressed miRNAs (FDR (false discovery rate)<0.05) and with a log FC (log fold change)>1.5 are shown for: A) OPC vs. control HPV-negative; C) OPC vs control HPV-positive; and E) control HPV-negative vs. control HPV-positive. Heat map clustering of differentially expressed miRNAs (FDR<0.05) were plotted for: B) OPC vs. control HPV-negative; D) OPC vs. control HPV-positive; and F) control HPV-negative vs. control HPV-positive pairwise comparisons.

FIG. 2 is a diagrammatic representation showing one-way Kruskal-Wallis and Steel-Dwass pairwise comparison of salivary miRNA expression—miScript™ primer PCR Assays.

FIG. 3 is a graphical representation showing HPV-positive OPC vs HPV-positive controls; performance evaluation of miRNA diagnostic panel (A) ROC analysis, (B) Active parameter estimates considered in the panel, (C) Leave One Out Cross-Validation Mosaic Plot, (D) Leave One Out Cross-Validation Contingency Table.

FIG. 4 is a graphical representation showing HPV-negative OPC vs HPV-negative controls; performance evaluation of miRNA diagnostic panel (A) ROC analysis, (B) Active parameter estimates considered in the panel, (C) Leave One Out Cross-Validation Mosaic Plot, (D) Leave One Out Cross-Validation Contingency Table.

FIG. 5 is a graphical representation showing OPC (HPV-positive and -negative) vs Controls (HPV-positive and -negative); performance evaluation of miRNA diagnostic panel (A) ROC analysis, (B) Active parameter estimates considered in the panel, (C) Leave One Out Cross-Validation Mosaic Plot, (D) Leave One Out Cross-Validation Contingency Table.

FIG. 6 is a graphical representation showing a Kaplan Meier estimate of OPC patient survival by salivary expression of Hsa-miR-07-5p. Median Split cut-off of linear predictor, cut-off=1.246.

FIG. 7 is a graphical representation depicting a one-way Kruskal-Wallis analysis and Steel-Dwass pairwise comparison of salivary miRNA expression—Revalidation using miRCURY™ LNA primer PCR assays.

FIG. 8 is a graphical representation depicting HPV-OPC vs HPV-positive controls; performance evaluation of miRNA diagnostic panel (A) ROC analysis (B) Active parameter estimates considered in the panel (C) ROC analysis—revalidation (D) Active parameter estimates considered in the panel—revalidation. (A,B) miScript™ primer PCR Assays (C,D) miRCURY™ LNA primer PCR assays.

FIG. 9 is a graphical representation depicting HPV-negative OPC vs HPV-negative controls; performance evaluation of miRNA diagnostic panel (A) ROC analysis (B) Active parameter estimates considered in the panel (A,B) miScript™ primer PCR Assays.

FIG. 10 is a graphical representation depicting OPC (HPV-positive and negative) vs Controls (HPV-positive and negative); performance evaluation of miRNA diagnostic panel (A) ROC analysis (B) Active parameter estimates considered in the panel (C) ROC analysis-revalidation (D) Active parameter estimates considered in the panel—revalidation. (A,B) miScript™ primer PCR Assays (C,D) miRCURY™ LNA primer PCR assays.

Some figures and text contain color representations or entities. Color illustrations are available from the Applicant upon request or from an appropriate Patent Office. A fee may be imposed if obtained from a Patent Office.

### DETAILED DESCRIPTION

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, the term “HPV-agnostic OPC” refers to an OPC condition that is independent of HPV infection or non-infection.

The term “aiding diagnosis” is used herein to refer to methods that assist in making a clinical determination regarding the presence, or nature, of a particular type of symptom or condition of a disease or disorder (e.g., HPV-positive OPC, HPV-negative OPC or HPV-agnostic OPC). For example, a method of aiding diagnosis of a disease or condition as disclosed for example herein can comprise measuring certain biomarkers (e.g., the miRNA biomarkers disclosed herein) in a biological sample of an individual.

The “amount” or “level” of a biomarker is a detectable level or amount in a sample. These can be measured by methods known to one skilled in the art and also disclosed herein. These terms encompass a quantitative amount or level (e.g., weight or moles), a semi-quantitative amount or level, a relative amount or level (e.g., weight % or mole % within class), a concentration, and the like. Thus, these terms encompass absolute or relative amounts or levels or concentrations of a biomarker in a sample. The expression level or amount of biomarker assessed can be used to determine the response to treatment.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

As used herein, the term “amplicon” refers to a nucleic acid that is the product of amplification. Thus an amplicon may be homologous to a reference sequence, a target sequence, or any sequence of nucleic acid that has been subjected to amplification. Generally, within a reaction sample, the concentration of amplicon sequence will be significantly greater than the concentration of original (template) nucleic acid sequence.

As used herein, “and/or” refers to and encompasses any and all possible combinations of one or more of the associated listed items, as well as the lack of combinations when interpreted in the alternative (or).

The term “biomarker” as used herein refers to an indicator, e.g., predictive, diagnostic, and/or prognostic, which can be detected in a sample. The biomarker may serve as an indicator of a particular subtype of a disease or disorder (e.g., HPV-positive OPC, HPV-negative OPC and HPV-agnostic OPC, etc.), characterized by certain, molecular, pathological, histological, and/or clinical features, and/or may serve as an indicator of a particular cell type or state and/or or response to therapy. Biomarkers include, but are not limited to, polynucleotides (e.g., DNA, and/or RNA), polynucleotide copy number alterations (e.g., DNA copy numbers), polypeptides, polypeptide and polynucleotide modifications (e.g., posttranslational modifications), carbohydrates, and/or glycolipid-based molecular markers. A biomarker may be present in a sample obtained from a subject before the onset of a physiological or pathophysiological state (e.g., HPV-positive OPC, HPV-negative OPC and HPV-agnostic OPC, etc.), including a symptom, thereof (e.g., sore throat that does not go away, a lump in the throat, mouth or neck, trouble swallowing, trouble opening the mouth fully, trouble moving the tongue, weight loss for no reason, ear pain, a white patch on the tongue or lining of the mouth that does not go away, coughing up blood, etc.). Thus, the presence of the biomarker in a sample obtained from the subject can be indicative of an increased likelihood that the subject has or will develop the physiological or pathophysiological state or symptom thereof. Alternatively, or in addition, the biomarker may be normally expressed in an individual, but its expression may change (i.e., it is increased (upregulated; over-expressed) or decreased (downregulated; under-expressed) before the onset of a physiological or pathophysiological state, including a symptom thereof. Thus, a change in the level of the biomarker may be indicative of an increased likelihood that the subject has or will develop the physiological or pathophysiological state or symptom thereof. Alternatively, or in addition, a change in the level of a biomarker may reflect a change in a particular physiological or pathophysiological state, or symptom thereof, in a subject, thereby allowing the nature (e.g., severity) of the physiological or pathophysiological state, or symptom thereof, to be tracked over a period of time. This approach may be useful in, for example, monitoring a treatment regimen for the purpose of assessing its effectiveness (or otherwise) in a subject. As herein described, reference to the level of a biomarker includes the concentration of a biomarker, or the level of expression of a biomarker, or the activity of the biomarker.

The term “biomarker value” refers to a value measured or functionalized for at least one corresponding biomarker of a subject and which is typically indicative of an abundance or concentration of a biomarker in a sample obtained from the subject. Thus, the biomarker values could be measured biomarker values, which are values of biomarkers measured for the subject. These values may be quantitative or qualitative. For example, a measured biomarker value may refer to the presence or absence of a biomarker or may refer to a level of a biomarker in a sample. The measured biomarker values can be values relating to raw or normalized biomarker levels (e.g., a raw, non-normalized biomarker level, or a normalized biomarker levels that is determined relative to an internal or external control biomarker level) and to mathematically transformed biomarker levels (e.g., a logarithmic representation of a biomarker level such as amplification amount, cycle time, etc.). Alternatively, the biomarker values could be functionalized biomarker values, which are values that have been functionalized from one or more measured biomarker values, for example by applying a function to the one or more measured biomarker values. Biomarker values can be of any appropriate form depending on the manner in which the values are determined. For example, the biomarker values could be determined using high-throughput technologies such as mass spectrometry, sequencing platforms, array and hybridization platforms, immunoassays, flow cytometry, or any combination of such technologies and in representative examples, the biomarker values relate to a level of activity or abundance of an expression product or other measurable molecule, quantified using a nucleic acid assay such as real-time polymerase chain reaction (RT-PCR), sequencing or the like. In the context of nucleic acid amplification assays such as PCR-based assays, the biomarker values can be in the form of amplification amounts, or cycle times, which are a logarithmic representation of the levels of the biomarker within a sample and which thus correspond to mathematical transformations of raw or normalized biomarker levels, as will be appreciated by persons skilled in the art. Thus, in situations in which mathematically transformed biomarker values are used as measured biomarker values, the expression “functionalized biomarker value” in the context, for example, of a ratio of levels of a pair of biomarkers in a sample obtained from a subject does not necessarily mean that the functionalized biomarker value is one that results from a division of one measured biomarker value by another measured biomarker value. Instead, the measured biomarker values can be combined using any suitable function, whereby the resulting functionalized biomarker value is one that corresponds to or reflects a ratio of non-normalized (e.g., raw) or normalized biomarker levels.

The terms “biomarker signature”, “signature”, “biomarker expression signature”, or “expression signature” are used interchangeably herein and refer to one or a combination of biomarkers whose expression is an indicator, e.g., predictive, diagnostic, and/or prognostic. The biomarker signature may serve as an indicator of a particular subtype of a disease or disorder (e.g., HPV-positive OPC, HPV-negative OPC, HPV-agnostic OPC, etc.) or symptom thereof (e.g., response to therapy, drug resistance, and/or disease burden) characterized by certain molecular, pathological, histological, and/or clinical features. In some embodiments, the biomarker signature is a “gene signature”. The term “gene signature” is used interchangeably with “gene expression signature” and refers to one or a combination of polynucleotides (e.g., miRNA genes) whose expression is an indicator, e.g., predictive, diagnostic, and/or prognostic. A biomarker signature may comprise at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 or more biomarkers. A biomarker signature can further comprise one or more controls or internal standards. In certain embodiments, a biomarker signature comprises at least one biomarker, or indication thereof, that serves as an internal standard. In other embodiments, a biomarker signature comprises an indication of one or more types of biomarkers. The term “indication” as used herein in this context merely refers to a situation where the biomarker signature contains symbols, data, abbreviations or other similar indicia for a biomarker, rather than the biomarker molecular entity itself. The term “biomarker signature” is also used herein to refer to a biomarker value or combination of at least two biomarker values, wherein individual biomarker values correspond to values of biomarkers that can be measured or functionalized from one or more subjects, which combination is characteristic of a discrete condition, stage of condition, subtype of condition or a prognosis for a discrete condition, stage of condition, subtype of condition. The term “signature biomarkers” is used to refer to a subset of the biomarkers that have been identified for use in a biomarker signature that can be used in performing a clinical assessment, such as to rule in or rule out a specific condition, different stages or severity of conditions, subtypes of different conditions or different prognoses. The number of signature biomarkers will vary, but is typically of the order of 16 or less (e.g., 16, 15, 14, 13, 12, 11, 10, 9, 8, 7, 6, 5, 4, 3, 2 or 1).

The term “clinical parameter”, as used herein, refers any clinical measure of a health or disease status of a subject, such as, without limitation, age, ethnicity, gender, diastolic blood pressure and systolic blood pressure, family history, height, weight, waist and hip circumference, body-mass index, resting heart rate, β-cell function, macrovascular function, microvascular function, atherogenic index, blood pressure, low-density lipoprotein/high-density lipoprotein ratio, intima-media thickness, soreness (e.g., throat, mouth or neck) lump presence (e.g., throat, mouth or neck), swallow function, mouth function, tongue function, weight loss, pain (e.g., ear), voice function, tissue discoloration (e.g., the tongue or lining of the mouth), hemoptysis, etc., as well as risk factors such as smoking and chewing tobacco, heavy alcohol use, a diet low in fruits and vegetables, drinking mate (i.e., a stimulant drink common in South America), chewing betel quid (i.e., a stimulant commonly used in parts of Asia), and being infected with human papillomavirus (HPV).

As used herein, the term “clinical sign”, or simply “sign”, refers to objective evidence of the presence of disease or condition (e.g., OPC) in a subject. Symptoms and/or signs associated with a particular disease or condition and the evaluation of such signs are routine and known in the art. Examples of signs of OPC sore throat that does not go away, a lump in the throat, mouth or neck, trouble swallowing, trouble opening the mouth fully, trouble moving the tongue, weight loss for no reason, a change in voice, ear pain, a white patch on the tongue or lining of the mouth that does not go away, and coughing up blood.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “composite score” refers to an aggregation of the obtained values for biomarkers measured in a sample from a subject optionally in combination with one or more patient clinical parameters or signs. In some embodiments, the obtained biomarker values are normalized to provide a composite score for each subject tested. When used in the context of a risk categorization table and correlated to a stratified population grouping or cohort population grouping based on a range of composite scores in a risk categorization table, the “biomarker composite score” may be used, at least in part, by a machine learning system to determine the “risk score” for each subject tested wherein the numerical value (e.g., a multiplier, a percentage, etc.) indicating increased likelihood of having an OPC condition for the stratified grouping becomes the “risk score”.

As used herein, the term “correlates” or “correlates with” and like terms, refers to a statistical association between two or more things, such as events, characteristics, outcomes, numbers, data sets, etc., which may be referred to as “variables”. It will be understood that the things may be of different types. Often the variables are expressed as numbers (e.g., measurements, values, likelihood, risk), wherein a positive correlation means that as one variable increases, the other also increases, and a negative correlation (also called anti-correlation) means that as one variable increases, the other variable decreases. In various embodiments, correlating a biomarker or biomarker signature with the presence or absence of a condition (e.g., a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition), or with a survival prognosis (e.g., decreased or poor survival prognosis, or increased or good survival prognosis) comprises determining the presence, absence, level or amount of at least one miRNA biomarker in a subject that has that condition or prognosis; or in persons known to be free of that condition or prognosis. In specific embodiments, a profile of biomarker levels, absences or presences is correlated to a global probability or a particular outcome, using receiver operating characteristic (ROC) curves.

The term “cut-off value” as used herein is a level (or concentration) which may be an absolute level or a relative level, which is indicative of whether a subject has a particular disease or condition (e.g., a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition), or is at risk of having a particular disease or condition (e.g., a condition selected from HPV-positive OPC, OPC-negative HPV infection, HPV-negative OPC and HPV-agnostic OPC), or has a particular survival prognosis (e.g., decreased or poor survival prognosis, or increased or good survival prognosis). Depending on the biomarker or combination of biomarkers, a subject is regarded as having the disease or condition, or being at risk of having the disease or condition, or having a particular prognosis, if either the level of the biomarker(s) detected and determined, respectively, is lower than the cut-off value, or the level of the biomarker(s) detected and determined, respectively, is higher than the cut-off value.

As used herein, the terms “detectably distinct” and “detectably different” are used interchangeably herein to refer to a signal that is distinguishable or separable by a physical property either by observation or by instrumentation. For example, a fluorophore is readily distinguishable either by spectral characteristics or by fluorescence intensity, lifetime, polarization or photo-bleaching rate from another fluorophore in a sample, as well as from additional materials that are optionally present. In certain embodiments, the terms “detectably distinct” and “detectably different” refer to a set of labels (such as dyes, suitably organic dyes) that can be detected and distinguished simultaneously.

As used herein, the phrase “developing a classifier” refers to using input variables to generate an algorithm or classifier capable of distinguishing between two or more states (e.g., a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition), or a survival prognosis (e.g., decreased or poor survival prognosis, or increased or good survival prognosis).

As used herein, the terms “diagnosis”, “diagnosing” and the like are used interchangeably herein to encompass determining the likelihood that a subject will develop a condition, or the existence or nature of a condition in a subject. These terms also encompass determining the severity of disease or episode of disease, as well as in the context of rational therapy, in which the diagnosis guides therapy, including initial selection of therapy, modification of therapy (e.g., adjustment of dose or dosage regimen), and the like. By “likelihood” is meant a measure of whether a subject with particular measured or functionalized biomarker values actually has a condition (or not) based on a given mathematical model. An increased likelihood for example may be relative or absolute and may be expressed qualitatively or quantitatively. For instance, an increased likelihood may be determined simply by determining the subject's measured biomarker values for at least 1, 2, 3, 4, 5, 6 or 7 biomarkers and placing the subject in an “increased likelihood” category, based upon previous population studies. The term “likelihood” is also used interchangeably herein with the term “probability”. The term “risk” relates to the possibility or probability of a particular event occurring at some point in the future. “Risk stratification” refers to an arraying of known clinical risk factors to allow physicians to classify patients into a low, moderate, high or highest risk of having or developing a particular disease or condition.

The term “differentially expressed” refers to differences in the quantity and/or the frequency of a biomarker present in a sample obtained from patients having, for example, a first condition (e.g., HPV-positive OPC) as compared to subjects with a second condition (e.g., HPV-negative OPC). For example, a biomarker can be a polynucleotide (e.g., miRNA) which is present at an elevated level or at a decreased level in samples of patients with HPV-positive OPC compared to samples of subjects with HPV-negative OPC. Alternatively, a biomarker can be a polynucleotide (e.g., miRNA) which is detected at a higher frequency or at a lower frequency in samples of patients with a first condition (e.g., HPV-negative OPC) compared to samples of subjects with a second condition (e.g., an OPC-negative, HPV-negative condition). A biomarker can be differentially present in terms of quantity, frequency or both.

The term “discrimination performance” refers to numeric representation of the index including, for example, sensitivity, specificity, positive predictability, negative predictability or accuracy. The term “discrimination performance” may also refer to a value computed by the functions of the indexes. For example, sensitivity, specificity, positive predictive value, negative predictive value and accuracy may each be used as the discrimination performance, or alternatively, the sum of two or more indexes, e.g., the sum of sensitivity and specificity, the sum of sensitivity and positive predictive value, or the sum of negative predictive value and accuracy, may be used as the discrimination performance.

The term “expression product”, as used herein, refers to any product produced during the process of gene expression including polynucleotide products and polypeptide products.

“Fluorophore” as used herein to refer to a moiety that absorbs light energy at a defined excitation wavelength and emits light energy at a different defined wavelength. Examples of fluorescence labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red and Texas Red-X.

The term “gene”, as used herein, refers to a stretch of nucleic acid that codes for a polypeptide or for an RNA chain that has a function. While it is the exon region of a gene that is transcribed to form mRNA, the term “gene” also includes regulatory regions such as promoters and enhancers that govern expression of the exon region.

As used herein, the term “gene expression profiling” is used in the broadest sense, and includes methods of quantification of RNA (e.g., mRNA, miRNA, and/or protein levels in a biological sample.

As used herein, the term “higher” with reference to a biomarker measurement refers to a statistically significant and measurable difference in the level of a biomarker compared to the level of another biomarker or to a control level where the biomarker measurement is greater than the level of the other biomarker or the control level. The difference is suitably at least about 10%, or at least about 20%, or of at least about 30%, or of at least about 40%, or at least about 50%.

As used herein the terms “homology”, “homologous” and the like refer to the level of similarity between two or more nucleic acid sequences in terms of percent of sequence identity. Generally, homologous sequences or sequences with homology refer to nucleic acid sequences that exhibit at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to one another. Alternatively, or in addition, homologs, homologous sequences or sequences with homology refer to nucleic acid sequences that hybridize under high stringency conditions to one another. High stringency conditions include and encompass from at least about 31% v/v to at least about 50% v/v formamide and from at least about 0.01 M to at least about 0.15 M salt for hybridization at 42° C., and at least about 0.01 M to at least about 0.15 M salt for washing at 42° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

The term “immobilized” means that a molecular species of interest is fixed to a solid support, suitably by covalent linkage. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physicochemical interactions typically occur in interactions between molecules. In particular embodiments, all that is required is that the molecules (e.g., nucleic acid molecules) remain immobilized or attached to a support under conditions in which it is intended to use the support, for example in applications requiring nucleic acid amplification and/or sequencing. For example, oligonucleotides or primers are immobilized such that a 3′ end is available for enzymatic extension and/or at least a portion of the sequence is capable of hybridizing to a complementary sequence. In some embodiments, immobilization can occur via hybridization to a surface attached primer, in which case the immobilized primer or oligonucleotide may be in the 3′-5′ orientation. In other embodiments, immobilization can occur by means other than base-pairing hybridization, such as the covalent attachment.

As used herein, the term “increase” or “increased” with reference to a biomarker level refers to a statistically significant and measurable increase in the biomarker level compared to the level of another biomarker or to a control level. The increase is suitably an increase of at least about 10%, or an increase of at least about 20%, or an increase of at least about 30%, or an increase of at least about 40%, or an increase of at least about 50%.

The term “indicator” as used herein refers to a result or representation of a result, including any information, number (e.g., biomarker value including functionalized biomarker value and composite score), ratio, signal, sign, mark, or note by which a skilled artisan can estimate and/or determine a likelihood or risk of whether or not a subject is suffering from a given disease or condition. In the case of the present invention, the “indicator” may optionally be used together with other clinical characteristics, to arrive at a diagnosis (that is, the occurrence or nonoccurrence) of an OPC condition disclosed herein, or a prognosis for a disclosed OPC condition in a subject. That such an indicator is “determined” is not meant to imply that the indicator is 100% accurate. The skilled clinician may use the indicator together with other clinical parameters or signs to arrive at a diagnosis.

The term “label” is used herein in a broad sense to refer to an agent that is capable of providing a detectable signal, either directly or through interaction with one or more additional members of a signal producing system and that has been artificially added, linked or attached via chemical manipulation to a molecule. Labels can be visual, optical, photonic, electronic, acoustic, optoacoustic, by mass, electro-chemical, electro-optical, spectrometry, enzymatic, or otherwise chemically, biochemically hydrodynamically, electrically or physically detectable. Labels can be, for example tailed reporter, marker or adapter molecules. In specific embodiments, a molecule such as a nucleic acid molecule is labeled with a detectable molecule selected form the group consisting of radioisotopes, fluorescent compounds, bioluminescent compounds, chemiluminescent compounds, metal chelators or enzymes. Examples of labels include, but are not limited to, the following radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin, e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags).

As used herein, the term “lower” with reference to a biomarker measurement refers to a statistically significant and measurable difference in the level of a biomarker compared to the level of another biomarker or to a control level where the biomarker measurement is less than the level of the other biomarker or the control level. The difference is suitably at least about 10%, or at least about 20%, or of at least about 30%, or of at least about 40%, or at least about 50%.

The term “microarray” refers to an arrangement of array elements, e.g., probes (including primers), ligands, biomarker nucleic acid sequence or protein sequences on a substrate. The term “microarray” includes within its scope “high-density arrays” and “low-density arrays”. In specific embodiments, the microarray refers to a substrate or collection of substrates or surfaces bearing a plurality of array elements (e.g., discrete regions having particular moieties, e.g., proteins (e.g., antibodies), nucleic acids (e.g., oligonucleotide probes), etc., immobilized thereto), where the array elements are present at a density of about 100 elements/cm² or more, about 1,000 elements/cm² or more, about 10,000 elements/cm² or more, or about 100,000 elements/cm² or more. In specific embodiments, a “high-density array” is one that comprises a plurality of array elements for detecting about 100 or more different biomarkers, about 1,000 or more different biomarkers, about 10,000 or more different biomarkers, or about 100,000 or more different biomarkers. In representative example of these embodiments, a “high-density array” is one that comprises a plurality of array elements for detecting biomarkers of about 100 or more different genes, of about 1,000 or more different genes, of about 10,000 or more different genes, or of about 100,000 or more different genes. Generally, the elements of a high-density array are not labeled. The term “low-density array” refers to a substrate or collection of substrates or surfaces bearing a plurality of array elements (e.g., discrete regions having particular moieties, e.g., proteins (e.g., antibodies), nucleic acids (e.g., oligonucleotide probes), etc., immobilized thereto), where the array elements are present at a density of about 100 elements/cm² or less, about 50 elements/cm² or less, about 20 elements/cm² or less, or about 10 elements/cm² or less. In specific embodiments, a “low-density array” is one that comprises a plurality of array elements for detecting about 100 or less different biomarkers, about 50 or less different biomarkers, about 20 or less different biomarkers (e.g., 20, 19, 18, 17, 16, 15, 14, 13, 12, 11, 10, 9, 8, 7, 6, 5, 4, 3, 2 or 1 distinct biomarker(s)), or about 10 or less different biomarkers (e.g., 10, 9, 8, 7, 6, 5, 4, 3, 2 or 1 distinct biomarker(s)). In representative example of these embodiments, a “low-density array” is one that comprises a plurality of array elements for detecting biomarkers of about 100 or less different genes, of about 50 or less different genes, of about 20 or less different genes (e.g., 20, 19, 18, 17, 16, 15, 14, 13, 12, 11, 10, 9, 8, 7, 6, 5, 4, 3, 2 or 1 distinct gene(s)), or of about 10 or less different genes (e.g., 10, 9, 8, 7, 6, 5, 4, 3, 2 or 1 distinct gene(s)). Generally, the elements of a low-density array are not labeled.

As used herein, “microRNA” or “miRNA” refers to a non-coding RNA, typically between about 18 and 25 nucleotides in length that hybridizes to and regulates the expression of a coding RNA. In certain embodiments, a miRNA is the product of cleavage of a precursor (pre-miRNA), for example by the enzyme Dicer. As used herein, “pre-miRNA” refers to a non-coding RNA having a hairpin structure, which contains a miRNA. Typically the term “pre-miRNA” refers to a precursor molecule, the processing and cleavage of which gives rise to a mature miRNA. In certain embodiments, a pre-miRNA is the product of cleavage of a pri-miR by a double-stranded RNA-specific ribonuclease.

As used herein, the term “normalization” and its derivatives, when used in conjunction with measurement of biomarkers across samples and time, refer to mathematical methods, including but not limited to multiple of the median (MoM), standard deviation normalization, sigmoidal normalization, etc., where the intention is that these normalized values allow the comparison of corresponding normalized values from different datasets in a way that eliminates or minimizes differences and gross influences.

The term “nucleic acid” or “polynucleotide” as used herein includes RNA, mRNA, miRNA, cRNA, cDNA, mtDNA, or DNA. The term typically refers to a polymeric form of nucleotides of at least 10 bases in length, either ribonucleotides or deoxynucleotides or a modified form of either type of nucleotide. The term includes single and double stranded forms of DNA or RNA. It also includes within its scope products of amplification, synthetically created, products of reverse transcription of RNA or naturally occurring. In some embodiments, nucleic acid can be composed of non-naturally occurring nucleotides and/or modifications to naturally occurring nucleotides. Examples include, but are not limited to: phosphorylation of 5′ or 3′ nucleotides to allow for ligation or prevention of exonuclease degradation/polymerase extension, respectively; amino, thiol, alkyne, or biotinyl modifications for covalent and near covalent attachments; fluorophores and quenchers; phosphorothioate, methylphosphonates, phosphoroamidates and phosphotriester linkages between nucleotides to prevent degradation; methylation; and modified bases or nucleosides such as deoxy-inosine, 5-bromo-dU, 2′-deoxy-uridine, 2-aminopurine, 2′,3′-dideoxy-cytidine, 5-methyl-dC, locked nucleic acids (LNAs), iso-dC and dG bases, 2′-O-methyl RNA bases and fluorine modified nucleosides.

By “obtained” is meant to come into possession. Samples so obtained include, for example, nucleic acid extracts or polypeptide extracts isolated or derived from a particular source. For instance, the extract may be isolated directly from a biological fluid or tissue of a subject.

“Oropharyngeal cancer” or “OPC” is a disease in which malignant (cancer) cells form in the tissues of the oropharynx. The oropharynx is the middle part of the pharynx (throat) behind the mouth, and includes the back one-third of the tongue, the soft palate, the side and back walls of the throat, and the tonsils. The pharynx is a hollow tube about 5 inches long that starts behind the nose and ends at the top of the trachea (windpipe) and esophagus (the tube that goes from the throat to the stomach). Air and food pass through the pharynx on the way to the trachea or the esophagus. Most oropharyngeal cancers are squamous cell carcinomas. Squamous cells are the thin, flat cells that line the inside of the oropharynx.

As used herein, the term “panel” refers to specific combination of biomarkers used to determine an indicator for assessing a likelihood that a condition as disclosed herein is present or absent in a subject. The term “panel” may also refer to an assay comprising a set of biomarkers used for such a determination. This term can also refer to a profile or index of expression patterns of one or more biomarkers described herein. The number of biomarkers useful for a biomarker panel is based on the sensitivity and specificity value for the particular combination of biomarker values.

As used herein, the term “positive response” means that the result of a treatment regimen includes some clinically significant benefit, such as the prevention, or reduction of severity, of symptoms, or a slowing of the progression of the condition. By contrast, the term “negative response” means that a treatment regimen provides no clinically significant benefit, such as the prevention, or reduction of severity, of symptoms, or increases the rate of progression of the condition.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the primer may be at least about 5, 6, 7, 8, 9, 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, to one base shorter in length than the template sequence at the 3′ end of the primer to allow extension of a nucleic acid chain, though the 5′ end of the primer may extend in length beyond the 3′ end of the template sequence. In certain embodiments, primers can be large polynucleotides, such as from about 35 nucleotides to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Desirably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

As used herein, the term “probe” refers to a molecule that binds to a specific sequence or sub-sequence or other moiety of another molecule. Unless otherwise indicated, the term “probe” typically refers to a nucleic acid probe that binds to another nucleic acid, also referred to herein as a “target polynucleotide”, through complementary base pairing. Probes can bind target polynucleotides lacking complete sequence complementarity with the probe, depending on the stringency of the hybridization conditions. Probes can be labeled directly or indirectly and include primers within their scope.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The skilled artisan will understand that the term “prognosis” refers to an increased probability that a certain course or outcome (e.g., survival) will occur; that is, that a course or outcome is more likely to occur in a subject exhibiting a given condition, when compared to those individuals not exhibiting the condition.

The term “proximal to” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. In general, the term indicates an element is located relatively near to the reference point than another element.

As used herein, the term “quencher” includes any moiety that in close proximity to a donor fluorophore, takes up emission energy generated by the donor fluorophore and either dissipates the energy as heat or emits light of a longer wavelength than the emission wavelength of the donor fluorophore. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan™ probes while proximal quenching is used in molecular beacon and Scorpion™ type probes. Suitable quenchers are selected based on the fluorescence spectrum of the particular fluorophore. Useful quenchers include, for example, the Black Hole™ quenchers BHQ-1, BHQ-2, and BHQ-3 (Biosearch Technologies, Inc.), and the ATTO-series of quenchers (ATTO 540Q, ATTO 580Q, and ATTO 612Q; Atto-Tec GmbH).

As used herein, a “reaction vessel” refers to any container, chamber, device, or assembly, in which a reaction can occur in accordance with the present disclosure. In some embodiments, a reaction vessel may be a microtube, for example, but not limited to, a 0.2 mL or a 0.5 mL reaction tube such as a MicroAmp™ Optical tube (Applied Biosystems™, Thermo Fisher Scientific) or a micro-centrifuge tube, or other containers of the sort in common practice in molecular biology laboratories. In some embodiments, a reaction vessel may be a well in a microtiter plate (e.g., 96-well plate, 384-well plate) such as a TaqMan™ Array plate (Applied Biosystems™; Thermo Fisher Scientific), a spot on a glass slide, a well in an Applied Biosystems™ TaqMan™ Array Card or Plate (Thermo Fisher Scientific) or a through-hole of an Applied Biosystems™ TaqMan™ OpenArray™ plate (Thermo Fisher Scientific). For example, a plurality of reaction vessels may reside on the same support. In some embodiments, lab-on-a-chip-like devices, available for example from Caliper, Fluidigm and Life Technologies Corp., including the Ion 316™ and Ion318™ Chip, may serve as reaction vessels in the disclosed methods and devices. In some embodiments, various microfluidic approaches may be employed. It will be recognized that a variety of reaction vessels are available in the art and fall within the scope of the present disclosure.

As used herein, the term “reduce” or “reduced” with reference to a biomarker level refers to a statistically significant and measurable reduction in the biomarker level compared to the level of another biomarker or to a control level. The reduction is suitably a reduction of at least about 10%, or a reduction of at least about 20%, or a reduction of at least about 30%, or a reduction of at least about 40%, or a reduction of at least about 50%.

The term “saliva sample” as used herein includes any biological specimen that may be extracted, untreated, treated, diluted or concentrated from a sample of saliva obtained from a subject. The term “saliva sample” includes saliva obtained from within the mouth, saliva obtained as spit, and saliva obtained from an oral rinse with a sampling fluid, such as sterile water.

The term “solid support” as used herein refers to a solid inert surface or body to which a molecular species, such as a nucleic acid can be immobilized. Non-limiting examples of solid supports include glass surfaces, plastic surfaces, latex, dextran, polystyrene surfaces, polypropylene surfaces, polyacrylamide gels, gold surfaces, and silicon wafers. In some embodiments, the solid supports are in the form of membranes, chips or particles. For example, the solid support may be a glass surface (e.g., a planar surface of a flow cell channel). In some embodiments, the solid support may comprise an inert substrate or matrix which has been “functionalized”, such as by applying a layer or coating of an intermediate material comprising reactive groups which permit covalent attachment to molecules such as polynucleotides. By way of non-limiting example, such supports can include polyacrylamide hydrogels supported on an inert substrate such as glass. The molecules (e.g., polynucleotides) can be directly covalently attached to the intermediate material (e.g., a hydrogel) but the intermediate material can itself be non-covalently attached to the substrate or matrix (e.g., a glass substrate). The support can include a plurality of particles or beads each having a different attached molecular species.

The terms “human subject”, “human individual”, “human patient”, “subject”, “individual” and “patient” are used interchangeably herein to refer to a normal healthy human individual and/or any human individual who may be at risk of OPC, or suffering from OPC, and/or has at least one clinical sign of OPC.

As used herein, the term “treatment regimen” refers to prophylactic and/or therapeutic (i.e., after onset of a specified condition) treatments, unless the context specifically indicates otherwise. The term “treatment regimen” encompasses natural substances and pharmaceutical agents (i.e., “drugs”) as well as any other treatment regimen including but not limited to dietary treatments, physical therapy or exercise regimens, surgical interventions, and combinations thereof.

It will be appreciated that the terms used herein and associated definitions are used for the purpose of explanation only and are not intended to be limiting.

### 2. Salivary miRNA Biomarkers for Discriminating Between HPV-Positive OPC, HPV-Negative OPC, HPV-Agnostic OPC and OPC-Negative Individuals

Disclosed herein are methods, compositions, apparatuses, devices and kits for aiding in distinguishing between subjects with HPV-positive OPC and OPC-negative, HPV-positive subjects, or between subjects with HPV-negative OPC and OPC-negative, HPV-negative subjects, or between subjects with HPV-agnostic OPC and OPC-negative subjects. Additionally, methods, compositions, apparatuses, devices and kits for aiding in predicting survival prognosis of OPC patients are disclosed. These methods, compositions, apparatuses, devices and kits are useful for early detection of HPV-positive OPC, OPC-negative HPV infection, HPV-negative OPC, HPV-agnostic OPC, OPC-negative, HPV-negative condition, OPC-negative condition and OPC-negative, HPV-negative condition, and predicting survival prognosis of OPC patients, thus allowing better triaging and/or treatment decisions for subjects with one or more clinical signs of OPC.

The present inventors have determined that certain miRNA biomarkers are commonly, specifically and differentially expressed in saliva samples obtained from different patient populations disclosed herein. The results presented herein provide clear evidence that specific miRNA biomarkers can be used, optionally in combination with clinical parameters or signs, to differentiate between HPV-positive OPC and OPC-negative HPV infection, or between HPV-negative OPC and an OPC-negative, HPV-negative condition, or between HPV-agnostic OPC and an OPC-negative condition, with a remarkable degree of accuracy. Additionally, it has been determined that a single miRNA biomarker can be used to predict decreased or poor patient survival, and may thus be useful for making better triaging decisions and management of affected patients.

MiRNA biomarkers that can be used in the practice of the methods, compositions, apparatuses, devices and kits disclosed herein include: (1) Hsa-miR-194-5p, Hsa-miR-501-3p and Hsa-miR-548K, which are differentially expressed in saliva of patients with HPV-positive OPC as compared to saliva of patients with OPC-negative HPV infection; (2) Hsa-miR-215-3p, Hsa-miR-194-5p, Hsa-miR-449a, Hsa-miR-07-5p, Hsa-miR-501-3p, Hsa-miR-1290 and Hsa-miR-548K, which are differentially expressed in saliva of patients with HPV-negative OPC as compared to saliva of OPC-negative, HPV-negative patients; (3) Hsa-miR-3614-5p, Hsa-miR-501-3p, Hsa-miR-1290 and Hsa-miR-1246, which are differentially expressed in saliva of patients with HPV-agnostic OPC as compared to saliva of OPC-negative patients; and (4) Hsa-miR-07-5p, which is differentially expressed in saliva of OPC-positive patients with decreased or poor survival prognosis as compared to saliva of OPC-positive patients with increased or good survival prognosis. Differential expression of one or more of these “salivary miRNA biomarkers” is useful therefore for providing an indicator that aids in the diagnosis of and in distinguishing between these conditions, or in survival prognosis.

In various embodiments, the methods, compositions, apparatuses, devices and kits of the present disclosure are used to provide an indicator that aids in the diagnosis of any one of the following conditions: HPV-positive OPC, OPC-negative HPV infection, HPV-negative OPC, HPV-agnostic OPC, OPC-negative, HPV-negative condition, OPC-negative condition and OPC-negative, HPV-negative condition (also referred to herein as the “disclosed conditions”) in a subject with at least one clinical sign of OPC. In other embodiments, the disclosed methods, compositions, apparatuses, devices and kits are used as an aid to screen asymptomatic patients who are at risk of OPC for the presence or absence of any one of the disclosed conditions. In illustrative examples of this type, the asymptomatic patients may be ones determined to be persistently HPV-positive in the oral cavity (e.g., over at least two consecutive years) and thus at risk of OPC. Alternatively, the present methods, compositions, apparatuses, devices and kits may be used in health and wellness embodiments as an aid to screen healthy individuals for the presence or absence of any one of the disclosed conditions.

In one aspect, methods are disclosed herein for determining an indicator used in assessing a likelihood that HPV-positive OPC or OPC-negative HPV infection is present or absent in a subject, wherein the methods comprise, consist or consist essentially of: (1) determining a miRNA biomarker value for at least one biomarker (e.g., 1 to 3 miRNA biomarkers, and all integer biomarkers in between) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-501-3p and Hsa-miR-548K; and (2) determining the indicator using the biomarker value(s). Methods are also disclosed herein for determining an indicator used in assessing a likelihood that HPV-negative OPC or an OPC-negative, HPV-negative condition is present or absent in a subject wherein the methods comprise, consist or consist essentially of: (1) determining a miRNA biomarker value for at least one biomarker (e.g., 1 to 7 miRNA biomarkers, and all integer biomarkers in between) in a saliva sample obtained from the subject, wherein a respective biomarker value is indicative of a level of a corresponding miRNA biomarker in the sample, wherein the at least one miRNA biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K; and (2) determining the indicator using the biomarker value(s). In addition, methods are disclosed herein for determining an indicator used in assessing a likelihood that HPV-agnostic OPC or an OPC-negative condition is present or absent in a subject, wherein the methods comprise, consist or consist essentially of: (1) determining a miRNA biomarker value for at least one biomarker (e.g., 1 to 4 miRNA biomarkers, and all integer biomarkers in between) in a saliva sample obtained from the subject, wherein a respective biomarker value is indicative of a level of a corresponding miRNA biomarker in the sample, wherein the at least one miRNA biomarker is selected from Hsa-miR-3614-5p, Hsa-miR-501-3p, Hsa-miR-1290 and Hsa-miR-1246; and (2) determining the indicator using the biomarker value(s). Moreover, methods are disclosed herein for determining an indicator used in assessing a likelihood of a human subject with OPC having a decreased or poor survival prognosis, wherein the. These methods generally comprise, consist or consist essentially of methods comprise, consist or consist essentially of: (1) determining a biomarker value for Hsa-miR-07-5p in a saliva sample obtained from the subject, wherein the biomarker value is indicative of a level of Hsa-miR-07-5p in the sample; and (2) determining the indicator using the biomarker value.

Biomarker panels disclosed herein typically comprise at least 1 miRNA biomarker and up to 14 miRNA biomarkers, including any number of miRNA biomarkers in between, such as 2, 3, 4, 5, 6, 7, 8, 9, 10, 11, 12 and 13 miRNA biomarkers. In certain embodiments, an miRNA biomarker panel comprises at least 2, or least 3, or at least 4, or at least 5, or at least 6, or at least 7, or more miRNA biomarkers. In some embodiments, a miRNA biomarker panel comprises up to 3, or up to 4, or up to 5, or up to 6, or up to 7 miRNA biomarkers.

Biomarker values that are indicative of the levels of miRNA biomarkers in a patient sample may be obtained by any suitable means known in the art. A saliva sample can be saliva obtained from within the mouth, or obtained as spit. A saliva sample can also be a sample comprising saliva, as obtained by oral rinsing with a sampling rinse fluid, typically, e.g., sterile water, and then collecting the rinse, which then comprises saliva diluted with the rinse fluid.

Test samples of saliva may be obtained from a subject using any suitable means known in the art. The subject may have at least one clinical sign of OPC (e.g., sore throat that does not go away, a lump in the throat, mouth or neck, trouble swallowing, trouble opening the mouth fully, trouble moving the tongue, weight loss for no reason, ear pain, a change in voice, a white patch on the tongue or lining of the mouth that does not go away, and coughing up blood). Alternatively, the subject may be asymptomatic but at risk of OPC, illustrative examples of which include subjects having a history of smoking or oral tobacco use, drinking mate (i.e., a stimulant drink common in South America), chewing betel quid (i.e., a stimulant commonly used in parts of Asia), are HPV-positive, are HIV-positive, have had multiple sexual partners and/or engage in oral sex. In other embodiments, the subject is a healthy subject.

Methods of obtaining saliva samples may include but are not limited to forcible ejection from the subject's mouth (e.g., spitting), aspiration, or removal by a swab or other collection tool. In some embodiments, the saliva may be separated into cellular and non-cellular fractions by suitable methods (e.g., centrifugation). In some embodiments, nucleic acids may be extracted from the cellular or non-cellular fractions.

Variability in sample preparation of cell-containing samples can be corrected by normalizing the data by, for example, protein content or cell number. In certain embodiments, the sample may be normalized relative to the total protein content in the sample. Total protein content in the sample can be determined using standard procedures, including, without limitation, Bradford assay and the Lowry method. In some embodiments, the sample may be normalized relative to cell number.

Nucleic acids, including miRNA, can be obtained from a saliva sample using any suitable technique. The nucleic acid content may also be obtained from an extraction performed on a fresh or fixed biological sample. The sample can be treated with an agent to preserve the miRNA prior to further processing, e.g., cell lysis and extraction. Samples can include frozen samples collected for other purposes. Samples can be associated with relevant information such as age, gender, and clinical symptoms present in the subject; source of the sample; and methods of collection and storage of the sample.

Measurement of the expression level of a miRNA biomarker in the sample can be direct or indirect. For example, the abundance levels of miRNA can be directly quantitated. Alternatively, the amount of a miRNA biomarker can be determined indirectly by measuring abundance levels of cDNAs, amplified RNAs or DNAs, or by measuring quantities or activities of RNAs that are indicative of the expression level of the miRNA biomarker. The methods for measuring miRNA biomarkers in a saliva sample have many applications. For example, one or more miRNA biomarkers can be measured to aid in survival prognosis or diagnosis of HPV-positive OPC, OPC-negative HPV infection, HPV-negative OPC, HPV-agnostic OPC, OPC-negative, HPV-negative condition, OPC-negative condition and OPC-negative, HPV-negative condition, to determine the appropriate treatment for a subject, or to monitor responses in a subject to treatment.

Thus, a salivary sample comprising miRNA (also referred to herein as a salivary miRNA sample) is assessed to determine biomarker values, which reflect the level of expression of at least one salivary miRNA associated with any one of the conditions disclosed herein. Representative nucleic acid sequences for the miRNA biomarkers of the present disclosure are presented in TABLE A below:

TABLE A
Sequences of miRNAs

| | miRBase | | SEQ |
|---|---|---|---|
| | Accession | | ID |
| Target | Number | Sequence | NO: |
| Hsa-miR-215-3p | MIMAT0026476 | ucugucauuucu | 1 |
| | | uuaggccaaua | |
| Hsa-miR-194-5p | MIMAT0000460 | uguaacagcaac | 2 |
| | | uccaugugga | |
| Hsa-miR-126-3p | MIMAT0000445 | ucguaccgugag | 3 |
| | | uaauaaugcg | |
| Hsa-miR-449a | MIMAT0001541 | uggcaguguauu | 4 |
| | | guuagcuggu | |
| Hsa-miR-199a-5p | MIMAT0000231 | cccaguguucag | 5 |
| | | acuaccuguuc | |
| Hsa-miR-3614-5p | MIMAT0017992 | ccacuuggaucu | 6 |
| | | gaaggcugccc | |
| Hsa-miR-619-5p | MIMAT0026622 | gcugggauuaca | 7 |
| | | ggcaugagcc | |
| Hsa-miR-07-5p | MIMAT0000252 | uggaagacuagu | 8 |
| | | gauuuuguuguu | |
| Hsa-miR-3529-3p | MIMAT0022741 | aacaacaaaauc | 9 |
| | | acuagucuucca | |
| Hsa-miR-99A-3p | MIMAT0004511 | caagcucgcuuc | 10 |
| | | uaugggucug | |
| Hsa-miR-501-3p | MIMAT0004774 | aaugcacccggg | 11 |
| | | caaggauucu | |
| Hsa-miR-1290 | MIMAT0005880 | uggauuuuugga | 12 |
| | | ucaggga | |
| Hsa-miR-548K | MIMAT0005882 | aaaaguacuugc | 13 |
| | | ggauuuugcu | |
| Hsa-miR-1246 | MIMAT0005898 | aauggauuuuug | 14 |
| | | gagcagg | |

miRNA expression levels may be determined using any suitable technique. For example, hybridization methods, such as Northern analysis can be used (see Current Protocols in Molecular Biology, 2012, Ausubel, F. et al., eds., John Wiley & Sons, including all supplements). For example, the presence of a miRNA disclosed herein can be indicated by hybridization to a nucleic acid probe. In representative examples of this type, a hybridization sample is formed by contacting the saliva sample with at least one nucleic acid probe. An exemplary probe for detecting miRNA is a labeled nucleic acid probe capable of hybridizing to miRNA. The nucleic acid probe can be, for example, a full-length nucleic acid molecule, or a portion thereof, such as an oligonucleotide of at least 10, 15, or 25 nucleotides in length and sufficient to specifically hybridize under stringent conditions to appropriate miRNA. The hybridization sample is maintained under conditions which are sufficient to allow specific hybridization of the nucleic acid probe to a miRNA target of interest. Specific hybridization can be performed under high stringency conditions or moderate stringency conditions, as appropriate. In some embodiments, the hybridization conditions for specific hybridization are high stringency. Specific hybridization, if present, is then detected using standard methods. If specific hybridization occurs between the nucleic acid probe and a miRNA in the test sample, the sequence that is present in the nucleic acid probe is also present in the miRNA of the subject. More than one nucleic acid probe can also be used concurrently in this method. Specific hybridization of any one of the nucleic acid probes is indicative of the presence of the miRNA of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen et al., Bioconjugate Chemistry 1994; 5:1). The PNA probe can be designed to specifically hybridize to a nucleic acid sequence comprising at least one miRNA of interest. Hybridization of the PNA probe to a nucleic acid sequence is indicative of the presence of a miRNA of interest.

Direct sequence analysis can also be used to detect miRNAs of interest. A sample comprising nucleic acid can be used, and polymerase chain reaction (PCR) or other appropriate methods can be used to amplify all or a fragment of the nucleic acid, and/or its flanking sequences.

In some embodiments, arrays of oligonucleotide probes that are complementary to target nucleic acid sequences from a subject can be used to detect, identify and quantify miRNAs associated with endometriosis. For example, in some embodiments, an oligonucleotide array can be used. Oligonucleotide arrays typically comprise a plurality of different oligonucleotide probes that are coupled to a surface of a substrate in different pre-defined or addressed locations. These oligonucleotide arrays, also referred to as “Genechips”, have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See, Fodor et al., Science 1991; 251:767-777; Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261. After an oligonucleotide array is prepared, a sample containing miRNA is hybridized with the array and scanned for miRNAs. Hybridization and scanning are generally carried out by methods described herein and also in, e.g., Published PCT Application Nos. WO 92/10092 and WO 95/11995, and U.S. Pat. No. 5,424,186. In brief, a target miRNA sequence is amplified by suitable amplification techniques, e.g., reverse transcriptase polymerase chain reaction (RT-PCR). Typically, this involves the use of primer sequences that are complementary to the target miRNA. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Other methods of nucleic acid analysis can be used to detect miRNAs of interest. Representative methods include direct manual sequencing (Church and Gilbert, Proc. Natl. Acad. Sci. USA 1988; 81:1991-1995; Sanger et al., Proc. Natl. Acad. Sci. USA 1977; 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield et al., Proc. Natl. Acad. Sci. USA 1981; 86:232-236), mobility shift analysis (Orita et al., Proc. Natl. Acad. Sci. USA 1989; 86:2766-2770; Rosenbaum and Reissner, Biophys. Chem. 1987; 265:1275; Keen et al., Trends Genet. 1991; 7:5); RNase protection assays (Myers, et al., Science 1985; 230:1242); Luminex xMAP™ technology; high-throughput sequencing (HTS) (Gundry and Vijg, Mutat Res. 2011; doi: 10.1016/j.mrfmmm.2011.10.001); next-generation sequencing (NGS) (Voelkerding et al., Clinical Chemistry 2009; 55:641-658; Su et al., Expert Rev Mol Diagn. 2011; 11:333-343; Ji and Myllykangas, Biotechnol Genet Eng Rev. 2011; 27:135-158); and/or ion semiconductor sequencing (Rusk, Nature Methods 2011; doi:10.1038/nmeth.f.330; Rothberg et al., Nature 2011; 475:348-352). These and other methods, alone or in combination, can be used to detect and quantify at least one miRNA of interest in a saliva sample obtained from a subject.

In some embodiments, sequencing can be performed for quantifying miRNA Suitable sequencing technologies including DNA sequencing and RNA sequencing, such as Sanger sequencing, pyrosequencing, sequencing by ligation, massively parallel sequencing, also called “Next-generation sequencing” (NGS), whole transcriptome shotgun sequence (WTSS) (also referred to as “RNAseq”), nanopore sequencing, nanostring sequencing and other high-throughput sequencing approaches with or without sequence amplification of the target can also be used to detect or quantify the presence of salivary miRNA biomarkers in a sample. Sequence-based methods can provide further information regarding alternative splicing and sequence variation in previously identified genes. Sequencing technologies include a number of steps that are grouped broadly as template preparation, sequencing, detection and data analysis. Current methods for template preparation involve randomly breaking genomic DNA into smaller sizes from which each fragment is immobilized to a support. The immobilization of spatially separated fragment allows thousands to billions of sequencing reaction to be performed simultaneously. A sequencing step may use any of a variety of methods that are commonly known in the art. One specific example of a sequencing step uses the addition of nucleotides to the complementary strand to provide the DNA sequence. The detection steps range from measuring bioluminescent signal of a synthesized fragment to four-color imaging of single molecule. In some embodiments in which NGS is used to detect or quantify the presence of salivary miRNA biomarkers in a sample, the methods are suitably selected from semiconductor sequencing (Ion Torrent; Personal Genome Machine); Helicos True Single Molecule Sequencing (tSMS) (Harris et al. 2008, Science 320:106-109); 454 sequencing (Roche) (Margulies et al. 2005, Nature, 437, 376-380); SOLiD technology (Applied Biosystems); SOLEXA sequencing (Illumina); single molecule, real-time (SMRT™) technology of Pacific Biosciences; nanopore sequencing (Soni and Meller, 2007. Clin Chem 53: 1996-2001); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys). In some cases, the sequencing assay uses nanopore sequencing. In some cases, the sequencing assay includes some form of Sanger sequencing. In some cases, the sequencing involves shotgun sequencing; in some cases, the sequencing includes bridge PCR. In some cases, the sequencing is broad spectrum. In some cases, the sequencing is targeted.

Probes and primers that are specific to the miRNAs of the present disclosure can be labeled directly or indirectly with a radioactive or nonradioactive compound in order to obtain a detectable and/or quantifiable signal; the labeling of the primers or of the probes according to the disclosure is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxygenin, haptenes, dyes, and luminescent agents such as radioluminescent, chemiluminescent, bioluminescent, fluorescent or phosphorescent agents.

There are many suitable methods for the detection of specific nucleic acid sequences and new methods are continually reported. One such category of methods involves specific hybridization reactions as detailed below.

In the Northern blot, the nucleic acid probe is preferably labeled with a tag. That tag can be a radioactive isotope, a fluorescent dye or another conveniently detectable moiety. Another type of process for the specific detection of nucleic acids of exogenous organisms in a body sample are the hybridization methods as exemplified by U.S. Pat. Nos. 6,159,693 and 6,270,974, and related patents. To briefly summarize one of those methods, a nucleic acid probe of at least 10 nucleotides, preferably at least 15 nucleotides, more preferably at least 25 nucleotides, having a sequence complementary to a region of the target nucleic acid of interest is hybridized in a sample, subjected to depolymerizing conditions, and the sample is treated with an ATP/luciferase system, which will luminesce if the nucleic sequence is present.

In quantitative Northern blotting, levels of the polymorphic nucleic acid can be compared to wild-type levels of the nucleic acid.

Alternatively, miRNA is quantified using a template-dependent nucleic acid amplification technique. Numerous nucleic acid amplification processes are available to amplify the miRNA biomarkers of the present disclosure. For example, the amplification may be performed with a polymerase, e.g., in one or more PCRs. Amplification may be performed using other suitable methods. These methods often depend on the product catalyzed formation of multiple copies of a nucleic acid or its complement. One of such methods is polymerase chain reaction (PCR), including AFLP (amplified fragment length polymorphism) PCR, allele-specific PCR, Alu PCR, assembly, asymmetric PCR, colony PCR, helicase dependent PCR, hot start PCR, inverse PCR, in situ PCR, intersequence-specific PCR or IS SR PCR, digital PCR, droplet digital PCR, linear-after-the-exponential-PCR or Late PCR, long PCR, nested PCR, real-time PCR, duplex PCR, multiplex PCR, quantitative PCR, or single cell PCR. Other amplification methods may also be used, including ligase chain reaction (LCR), nucleic acid sequence-based amplification (NASBA), linear amplification, isothermal linear amplification, Qβ-replicase method, 3SR, Transcription Mediated Amplification (TMA), Strand Displacement Amplification (SDA), or Rolling Circle Amplification (RCA).

In preferred embodiments, PCR is employed for miRNA quantification, which is described for example in U.S. Pat. Nos. 4,683,195, 4,683,202, and 4,800,159. To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product.

In PCR, the nucleic acid probe can be labeled with a tag as discussed before. Most preferably the detection of the duplex is done using at least one primer directed to the target nucleic acid. In some embodiments of PCR, the detection of the hybridized duplex comprises electrophoretic gel separation followed by dye-based visualization.

Nucleic acid amplification procedures by PCR are described in e.g. U.S. Pat. No. 4,683,202. Briefly, the primers anneal to the target nucleic acid at sites distinct from one another and in an opposite orientation. A primer annealed to the target sequence is extended by the enzymatic action of a heat stable polymerase. The extension product is then denatured from the target sequence by heating, and the process is repeated. Successive cycling of this procedure on both strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

Stem-loop RT-PCR is a PCR method that is useful in the methods of the disclosure to amplify and quantify miRNAs of interest (See Caifu et al., Nucleic Acids Research 2005; 33:e179; Mestdagh et al., Nucleic Acids Research 2008; 36:e143; Varkonyi-Gasic et al., Methods Mol Biol. 2011; 744:145-57). Briefly, the method includes two steps: RT and real-time PCR. First, a stem-loop RT primer is hybridized to a miRNA molecule and then reverse transcribed with a reverse transcriptase. Then, the RT products are quantified using conventional real-time PCR.

The primers and/or probes used in nucleic acid amplification are generally designed to specifically hybridize under stringent conditions where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods. In some embodiments, the disclosure includes a primer that is complementary to a nucleic acid sequence of the miRNA of interest, and more particularly the primer includes 12 or more contiguous nucleotides substantially complementary to the sequence of the miRNA of interest. A primer featured in the disclosure suitably includes a nucleotide sequence sufficiently complementary to hybridize to a nucleic acid sequence of about 12 to 25 nucleotides. Typically, the primer differs by no more than 1, 2, or 3 nucleotides from the target miRNA nucleotide sequence. In some embodiments, the length of the primer varies in length, and is suitably from about 15 to 28 nucleotides in length (e.g., 15, 16, 17, 18, 19, 20, 21, 22, 23, 24, 25, 26, 27 or 28 nucleotides in length).

Hybridization is typically accomplished by annealing the oligonucleotide probe or primer to the template nucleic acid under conditions of stringency that prevent non-specific binding but permit binding of this template nucleic acid which has a significant level of homology with the probe or primer.

Among the conditions of stringency is the melting temperature (Tm) for the amplification step using the set of primers, which is in the range of about 50° C. to about 95° C. Typical hybridization and washing stringency conditions depend in part on the size (i.e., number of nucleotides in length) of the template nucleic acid or the oligonucleotide probe, the base composition and monovalent and divalent cation concentrations (Ausubel et al., 1994-1998, eds Current Protocols in Molecular Biology).

In some embodiments, the level of expression of miRNA in a sample is determined by real-time amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

Real-time amplification, such as real-time PCR, can be performed in various different iterations; it can be performed e.g. using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present disclosure is the Tm, which is in the range of about 50° C. to 95° C. Suitably, the Tm for any one of the hydrolysis-probes of the present disclosure is in the range of about 55° C. to about 80° C. In specific embodiments, the Tm applied for any one of the hydrolysis-probes of the present disclosure is about 75° C.

In certain advantageous embodiments, the template-dependent amplification involves quantification of transcripts in real-time. For example, RNA or DNA may be quantified using the Real-Time PCR (RT-PCR) technique (Higuchi, 1992, et al., Biotechnology 10: 413-417). By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundance of the specific RNA (e.g., mRNA or miRNA) from which the target sequence was derived can be determined for the respective tissues or cells. This direct proportionality between the concentration of the PCR products and the relative RNA (e.g., mRNA or miRNA) abundance is only true in the linear range of the PCR reaction. The final concentration of the target DNA in the plateau portion of the curve is determined by the availability of reagents in the reaction mix and is independent of the original concentration of target DNA. In some embodiments, multiplexed, tandem PCR (MT-PCR) is employed, which uses a two-step process for gene expression profiling from small quantities of RNA or DNA, as described for example in US Pat. Appl. Pub. No. 20070190540. In the first step, RNA is converted into cDNA and amplified using multiplexed gene specific primers. In the second step each individual gene is quantitated by RT-PCR. Real-time PCR is typically performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments of RT-PCR assays, a TaqMan™ probe is used for quantitating nucleic acid. Such assays may use energy transfer (“ET”), such as fluorescence resonance energy transfer (“FRET”), to detect and quantitate the synthesized PCR product. Typically, the TaqMan™ probe comprises a fluorescent label (e.g., a fluorescent dye) coupled to one end (e.g., the 5′-end) and a quencher molecule is coupled to the other end (e.g., the 3-end), such that the fluorescent label and the quencher are in close proximity, allowing the quencher to suppress the fluorescence signal of the dye via FRET. When a polymerase replicates the chimeric amplicon template to which the fluorescent labeled probe is bound, the 5′-nuclease of the polymerase cleaves the probe, decoupling the fluorescent label and the quencher so that label signal (such as fluorescence) is detected. Signal (such as fluorescence) increases with each PCR cycle proportionally to the amount of probe that is cleaved.

TaqMan™ probes typically comprise a region of contiguous nucleotides having a sequence that is identically present in or complementary to a region of a disclosed miRNA biomarker such that the probe is specifically hybridizable to the resulting PCR amplicon. In some embodiments, the probe comprises a region of at least 6 contiguous nucleotides having a sequence that is fully complementary to or identically present in a region of a miRNA biomarker, such as comprising a region of at least 8 contiguous nucleotides, at least 10 contiguous nucleotides, at least 12 contiguous nucleotides, at least 14 contiguous nucleotides, or at least 16 contiguous nucleotides having a sequence that is complementary to or identically present in a region of a target miRNA biomarker to be detected and/or quantitated.

In addition to the TaqMan™ assays, other real-time PCR chemistries useful for detecting PCR products in the methods presented herein include, but are not limited to, Molecular Beacons, Scorpion probes and intercalating dyes, such as SYBR Green, EvaGreen, thiazole orange, YO-PRO, TO-PRO, etc. For example, Molecular Beacons, like TaqMan™ probes, use FRET to detect and quantitate a PCR product via a probe having a fluorescent label (e.g., a fluorescent dye) and a quencher attached at the ends of the probe. Unlike TaqMan™ probes, however, Molecular Beacons remain intact during the PCR cycles. Molecular Beacon probes form a stem-loop structure when free in solution, thereby allowing the fluorescent label and quencher to be in close enough proximity to cause fluorescence quenching. When the Molecular Beacon hybridizes to a target, the stem-loop structure is abolished so that the fluorescent label and the quencher become separated in space and the fluorescent label fluoresces. Molecular Beacons are available, e.g., from Gene Link™ (see, www.genelink.com).

In some embodiments, Scorpion probes can be used as both sequence-specific primers and for PCR product detection and quantitation. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent label (e.g., a fluorescent dye molecule) is attached to the 5′-end of the Scorpion probe, and a quencher is attached to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the fluorescent label on the 5′-end to fluoresce and generate a signal. Scorpion probes are available from, e.g., Premier Biosoft International (see www.premierbiosoft.com/tech notes/Scorpion.html).

In some embodiments, labels that can be used on the FRET probes include colorimetric and fluorescent dyes such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some embodiments, the same quencher may be used for multiple dyes, for example, a broad spectrum quencher, such as an Iowa Black™ quencher (Integrated DNA Technologies, Coralville, Iowa) or a Black Hole Quencher™ (BHQ™; Sigma-Aldrich, St. Louis, Mo.).

In some embodiments, for example, in a multiplex reaction in which two or more moieties (such as amplicons) are detected simultaneously, each probe comprises a detectably different dye such that the dyes may be distinguished when detected simultaneously in the same reaction. One skilled in the art can select a set of detectably different dyes for use in a multiplex reaction. In some embodiments, multiple target miRNA biomarkers are detected and/or quantitated in a single multiplex reaction. In some embodiments, each probe that is targeted to a different miRNA biomarker is spectrally distinguishable when released from the probe. Thus, each target miRNA biomarker is detected by a unique fluorescence signal.

Specific examples of fluorescently labeled ribonucleotides useful in the preparation of real-time PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of real-time PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Invitrogen.

In some embodiments, SAGE analysis is used to determine RNA abundances in a cell sample (see, e.g., Velculescu et al., Science 1995; 270:484-7; Carulli, et al., Journal of Cellular Biochemistry 1998; Supplements 30/31:286-96). SAGE analysis does not require a special device for detection, and is one of the preferable analytical methods for simultaneously detecting the expression of a large number of transcription products. First, poly A⁺ RNA is extracted from cells. Next, the RNA is converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a read-out of consecutive ditags of constant length. The presence of mRNA corresponding to each tag can then be identified from the nucleotide sequence of the clone and information on the sequence tags.

In some embodiments, amplification of miRNA is characterized in that the amplification products can be elongated, wherein the elongation products are separated relative to their length. The signal obtained for the elongation products is measured, and a quantitative and/or qualitative profile of the labeling intensity relative to the elongation product length is established. The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In non-limiting embodiments of the miRNA assays of the present disclosure, compositions are prepared for use in the indicator-determining methods disclosed herein. These compositions may comprise a mixture of a DNA polymerase (e.g., a thermostable DNA polymerase), saliva cDNA from a subject with least one clinical sign of OPC, wherein the saliva cDNA comprises at least one cDNA corresponding to a miRNA biomarker disclosed herein, and wherein the composition further comprises for each cDNA at least one oligonucleotide primer or probe that hybridizes to that cDNA. In some of the same or other embodiments, the compositions comprise for respective cDNA two oligonucleotide primers that hybridize to opposite complementary strands of the cDNA. In some of the same or other embodiments, the compositions comprise for a respective cDNA an oligonucleotide probe that hybridizes to the cDNA or a polynucleotide corresponding thereto (e.g., a polynucleotide product resulting from nucleic acid amplification of the cDNA). The oligonucleotide probe may comprise a heterologous label (e.g., a fluorescent label). In embodiments in which the oligonucleotide probe comprises a heterologous label, the labeled oligonucleotide probe may comprise a fluorophore. In representative examples of this type, the labeled oligonucleotide probe further comprises a quencher. In certain embodiments, different labeled oligonucleotide probes are included in the composition for hybridizing to different cDNAs, wherein individual oligonucleotide probes comprise detectably distinct labels (e.g. different fluorophores), or at least a subset of oligonucleotide probes comprises the same label (e.g. same fluorophore). In some embodiments, the compositions comprise for each of at least 2, 4, 5, 6, 7, or 8 of the cDNAs at least one oligonucleotide primer and/or probe that hybridizes to the cDNA. In other embodiments, the compositions comprise for each of up to 2, 4, 5, 6, 7, or 8 of the cDNAs at least one oligonucleotide primer and/or probe that hybridizes to the cDNA. Individual cDNAs and their corresponding oligonucleotide primer(s) and/or probe(s) may be present in separate reaction vessels or in the same reaction vessel.

### 2.1 Biomarker Panels

The present inventors have determined that certain salivary miRNA biomarkers have strong discrimination performance when combined with one or more other salivary miRNA biomarkers. In advantageous embodiments, specific combinations of salivary miRNA biomarkers have been identified that can be used to determine the indicator. Accordingly, in representative examples, an indicator is determined that correlates to biomarker values for a combination of salivary miRNA biomarkers, which can be used in assessing a likelihood that a condition selected from HPV-positive OPC, OPC-negative HPV-positive, HPV-negative OPC, HPV-agnostic OPC, OPC-negative, HPV-negative condition, OPC-negative condition and OPC-negative, HPV-negative condition is present or absent in a subject.

In these examples, the indicator-determining methods suitably include determining biomarker values for a plurality of miRNA biomarkers, wherein each biomarker value is a value measured for at least one corresponding miRNA biomarker of the subject and is indicative of a concentration of the miRNA biomarker in a saliva sample obtained from the subject. The biomarker values are typically used to determine a combined biomarker value (also referred to herein as a “composite score”) on which at least in part an indicator for assessing a likelihood that one of the conditions disclosed herein is present or absent in a subject is determined.

In some embodiments relating to discrimination between HPV-positive OPC and OPC-negative HPV infection, biomarker values are determined for a first miRNA biomarker, a second miRNA biomarker and a third miRNA biomarker, wherein the first miRNA biomarker is Hsa-miR-194-5p that is expressed at a lower level in HPV-positive OPC than in OPC-negative HPV infection, wherein the second miRNA biomarker is Hsa-miR-501-3p that is expressed at a lower level in HPV-positive OPC than in OPC-negative HPV infection, and wherein the third miRNA biomarker is Hsa-miR-548K that is expressed at a lower level in HPV-positive OPC than in OPC-negative HPV infection.

In some embodiments relating to discrimination between HPV-positive OPC and an OPC-negative, HPV-positive condition, biomarker values are determined for a first miRNA biomarker, a second miRNA biomarker, a third miRNA biomarker, a fourth miRNA biomarker, a fifth miRNA biomarker, a sixth miRNA biomarker, a seventh miRNA biomarker, an eighth miRNA biomarker and a ninth miRNA biomarker, wherein the first miRNA biomarker is Hsa-miR-194-5p that is expressed at a lower level in HPV-positive OPC than in OPC-negative, HPV positive condition, wherein the second miRNA biomarker is Hsa-miR-449a that is expressed at a lower level in HPV-positive OPC than in OPC-negative, HPV positive condition, wherein the third miRNA biomarker is Hsa-miR-3614-5p that is expressed at a lower level in HPV-positive OPC than in OPC-negative, HPV positive condition, wherein the fourth miRNA biomarker is Hsa-miR-07-5p that is expressed at a lower level in HPV-positive OPC than in OPC-negative, HPV positive condition, wherein the fifth miRNA biomarker is Hsa-miR-3529-3p that is expressed at a lower level in HPV-positive OPC than in OPC-negative, HPV positive condition, wherein the sixth miRNA biomarker is Hsa-miR-99A-3p that is expressed at a lower level in HPV-positive OPC than in OPC-negative, HPV positive condition, wherein the seventh miRNA biomarker is Hsa-miR-501-3p that is expressed at a lower level in HPV-positive OPC than in OPC-negative, HPV positive condition, wherein the eighth miRNA biomarker is Hsa-miR-1290 that is expressed at a lower level in HPV-positive OPC than in OPC-negative, HPV positive condition, and wherein the ninth miRNA biomarker is Hsa-miR-548K that is expressed at a lower level in HPV-positive OPC than in OPC-negative, HPV positive condition.

In some embodiments relating to distinguishing between HPV-negative OPC and OPC-negative, HPV-negative condition, biomarker values are determined for a first miRNA biomarker, a second miRNA biomarker, a third miRNA biomarker, a fourth miRNA biomarker, a fifth miRNA biomarker, a sixth miRNA biomarker and a seventh miRNA biomarker, wherein the first miRNA biomarker is Hsa-miR-215-3p that is expressed at a lower level in HPV-negative OPC than in OPC-negative, HPV-negative condition, wherein the second miRNA biomarker is Hsa-miR-194-5p, that is expressed at a lower level in HPV-negative OPC than in OPC-negative, HPV-negative condition, wherein the third miRNA biomarker is Hsa-miR-449a that is expressed at a lower level in HPV-negative OPC than in OPC-negative, HPV-negative condition, wherein the fourth miRNA biomarker is Hsa-miR-07-5p that is expressed at a lower level in HPV-negative OPC than in OPC-negative, HPV-negative condition, wherein the fifth miRNA biomarker is Hsa-miR-501-3p that is expressed at a lower level in HPV-negative OPC than in OPC-negative, HPV-negative condition, wherein the sixth miRNA biomarker is Hsa-miR-1290 that is expressed at a lower level in HPV-negative OPC than in OPC-negative, HPV-negative condition, and wherein the seventh miRNA biomarker is Hsa-miR-548K; that is expressed at a lower level in HPV-negative OPC than in OPC-negative, HPV-negative condition.

In some embodiments relating to distinguishing between HPV-negative OPC and OPC-negative, HPV-negative condition, biomarker values are determined for a first miRNA biomarker, a second miRNA biomarker, a third miRNA biomarker, a fourth miRNA biomarker, a fifth miRNA biomarker and a sixth miRNA biomarker, wherein the first miRNA biomarker is Hsa-miR-194-5p that is expressed at a lower level in HPV-negative OPC than in OPC-negative, HPV-negative condition, wherein the second miRNA biomarker is Hsa-miR-449a, that is expressed at a lower level in HPV-negative OPC than in OPC-negative, HPV-negative condition, wherein the third miRNA biomarker is Hsa-miR-07-5p that is expressed at a lower level in HPV-negative OPC than in OPC-negative, HPV-negative condition, wherein the fourth miRNA biomarker is Hsa-miR-501-3p that is expressed at a lower level in HPV-negative OPC than in OPC-negative, HPV-negative condition, wherein the fifth miRNA biomarker is Hsa-miR-1290 that is expressed at a lower level in HPV-negative OPC than in OPC-negative, HPV-negative condition, and wherein the sixth miRNA biomarker is Hsa-miR-548K that is expressed at a lower level in HPV-negative OPC than in OPC-negative, HPV-negative condition.

In some embodiments relating to differentiating between HPV-agnostic OPC and OPC-negative condition, biomarker values are determined for a first miRNA biomarker, a second miRNA biomarker, a third miRNA biomarker and a fourth miRNA biomarker, wherein the first miRNA biomarker is Hsa-miR-3614-5p that is expressed at a lower level in HPV-agnostic OPC than in OPC-negative condition, wherein the second miRNA biomarker is Hsa-miR-501-3p that is expressed at a lower level in HPV-agnostic OPC than in OPC-negative condition, wherein the third miRNA biomarker is Hsa-miR-1290 that is expressed at a lower level in HPV-agnostic OPC than in an OPC-negative condition, and wherein the fourth miRNA biomarker is Hsa-miR-1246 that is expressed at a lower level in HPV-agnostic OPC than in OPC-negative condition.

In some embodiments relating to differentiating between HPV-agnostic OPC and OPC-negative condition (e.g., HPV-positive, OPC-negative condition, HPV-negative, OPC-negative condition), biomarker values are determined for a first miRNA biomarker, a second miRNA biomarker, a third miRNA biomarker, a fourth miRNA biomarker, a fifth miRNA biomarker and a sixth miRNA biomarker, wherein the first miRNA biomarker is Hsa-miR-449a that is expressed at a lower level in HPV-agnostic OPC than in OPC-negative condition, wherein the second miRNA biomarker is Hsa-miR-3614-5p that is expressed at a lower level in HPV-agnostic OPC than in OPC-negative condition, wherein the third miRNA biomarker is Hsa-miR-07-5p that is expressed at a lower level in HPV-agnostic OPC than in an OPC-negative condition, wherein the fourth miRNA biomarker is Hsa-miR-501-3p that is expressed at a lower level in HPV-agnostic OPC than in OPC-negative condition, wherein the fifth miRNA biomarker is Hsa-miR-1290 that is expressed at a lower level in HPV-agnostic OPC than in OPC-negative condition, and wherein the sixth miRNA biomarker is Hsa-miR-548K that is expressed at a lower level in HPV-agnostic OPC than in OPC-negative condition.

In some embodiments relating to prognosis survival, a biomarker value is determined for Hsa-miR-07-5p, which is expressed at a lower level in patients with decreased or poor survival prognosis than in patients with increased or good prospects for survival.

The detection methods disclosed herein may further comprise applying a function to biomarker values to yield at least one functionalized biomarker value and determining the indicator using the at least one functionalized biomarker value. The function may include at least one of: (a) multiplying biomarker values; (b) dividing biomarker values; (c) adding biomarker values; (d) subtracting biomarker values; (e) a weighted sum of biomarker values; (f) a log sum of biomarker values; (g) a geometric mean of biomarker values; (h) a sigmoidal function of biomarker values; and (i) normalization of biomarker values.

In various embodiments employing panels of salivary miRNA biomarkers, the detection methods may further comprise combining the biomarker values to provide a composite score and determining the indicator using the composite score. Biomarker values may be combined by a combining function including, but not limited to, adding, multiplying, subtracting, and/or dividing biomarker values.

If desired, the detection methods may further comprise analyzing the biomarker value(s) or composite score with reference to a reference biomarker value, value range or threshold (e.g., cut-off) value, or composite score, score range or threshold value, to determine the indicator. For example, the indicator generally indicates a likelihood of a condition disclosed herein (e.g., HPV-positive OPC, OPC-negative HPV infection, HPV-negative OPC, HPV-agnostic OPC, OPC-negative, HPV-negative condition, OPC-negative condition and OPC-negative, HPV-negative condition) if the biomarker value(s) or composite score is indicative of the level of the biomarker(s) in the sample that correlates with an increased likelihood of a presence of the condition relative to a predetermined reference biomarker value, value range or cut-off value.

In some embodiments, a composite score is aggregated with one or more clinical parameters or signs to a composite score on which the indicator is determined.

### 2.2 Analysis of Biomarker Data

Biomarker data may be analyzed by a variety of methods to identify miRNA biomarkers and determine the statistical significance of differences in observed levels of miRNA biomarkers between test and reference expression profiles in order to evaluate whether a patient has a condition disclosed herein (e.g., HPV-positive OPC, OPC-negative HPV infection, HPV-negative OPC, HPV-agnostic OPC, OPC-negative, HPV-negative condition, OPC-negative condition and OPC-negative, HPV-negative condition) or to evaluate survival prognosis. For any particular miRNA biomarker, a distribution of miRNA biomarker levels for subjects with different conditions of the disclosure will likely overlap. Under such conditions, a test does not absolutely distinguish a first condition and a second condition with 100% accuracy, and the area of overlap indicates where the test cannot distinguish the first condition and the second condition. A threshold is selected, above which (or below which, depending on how miRNA biomarker changes with a specified condition or prognosis) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982)).

Alternatively, or in addition, thresholds may be established by obtaining an earlier miRNA biomarker result from the same patient, to which later results may be compared. In these embodiments, the individual in effect acts as their own “control group.” In miRNA biomarkers that decrease with condition severity or prognostic risk, a decrease over time in the same patient can indicate a worsening of the condition or a failure of a treatment regimen, while an increase over time can indicate remission of the condition or success of a treatment regimen.

In some embodiments, a positive likelihood ratio, negative likelihood ratio, odds ratio, and/or AUC or receiver operating characteristic (ROC) values are used as a measure of a method's ability to predict risk or to diagnose a condition disclosed herein (e.g., HPV-positive OPC, OPC-negative HPV infection, HPV-negative OPC, HPV-agnostic OPC, OPC-negative, HPV-negative condition, OPC-negative condition and OPC-negative, HPV-negative condition), or to prognose patient survival. As used herein, the term “likelihood ratio” is the probability that a given test result would be observed in a subject with a condition of interest divided by the probability that that same result would be observed in a patient without the condition of interest. Thus, a positive likelihood ratio is the probability of a positive result observed in subjects with the specified condition (e.g., HPV-positive OPC, OPC-negative HPV infection, HPV-negative OPC, HPV-agnostic OPC, OPC-negative, HPV-negative condition, OPC-negative condition or OPC-negative, HPV-negative condition), or survival prognosis, divided by the probability of a positive results in subjects without the specified condition or survival prognosis. A negative likelihood ratio is the probability of a negative result in subjects without the specified condition or survival prognosis divided by the probability of a negative result in subjects with specified condition or survival prognosis. The term “odds ratio,” as used herein, refers to the ratio of the odds of an event occurring in one group (e.g., one of the disclosed conditions or survival prognoses) to the odds of it occurring in another group (e.g., another of the disclosed conditions or survival prognoses), or to a data-based estimate of that ratio. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., one of the disclosed conditions or survival prognoses and another of the disclosed conditions or survival prognoses). ROC curves are useful for plotting the performance of a particular feature (e.g., any of the salivary miRNA biomarkers disclosed herein and/or any item clinical parameter or symptom information) in distinguishing or discriminating between two populations (e.g., one of the disclosed conditions or survival prognoses and another of the disclosed conditions or survival prognoses). Typically, the feature data across the entire population (e.g., subjects with one of the disclosed conditions or survival prognoses and subjects with another of the disclosed conditions or survival prognoses) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls. Although this definition refers to scenarios in which a feature is elevated in one patient group compared to another patient group, this definition also applies to scenarios in which a feature is lower in one patient group compared to the other patient group (in such a scenario, samples below the value for that feature would be counted). ROC curves can be generated for a single feature as well as for other single outputs, for example, a combination of two or more features (e.g., a combination of two or more biomarker values) can be mathematically combined (e.g., added, subtracted, multiplied, etc.) to produce a single value, and this single value can be plotted in a ROC curve. Additionally, any combination of multiple features (e.g., a combination of multiple biomarker values), in which the combination derives a single output value, can be plotted in a ROC curve. These combinations of features may comprise a test. The ROC curve is the plot of the sensitivity of a test against the specificity of the test, where sensitivity is traditionally presented on the vertical axis and specificity is traditionally presented on the horizontal axis. Thus, “AUC ROC values” are equal to the probability that a classifier will rank a randomly chosen positive instance higher than a randomly chosen negative one. An AUC ROC value may be thought of as equivalent to the Mann-Whitney U test, which tests for the median difference between scores obtained in the two groups considered if the groups are of continuous data, or to the Wilcoxon test of ranks.

In some embodiments, at least one (e.g., 1, 2, 3, 4, 5, 6, 7, 8, 9, 10, or more) miRNA biomarker or a panel of miRNA biomarkers is selected to discriminate between subjects with a first condition (e.g., one of HPV-positive OPC, OPC-negative HPV infection, HPV-negative OPC, HPV-agnostic OPC, OPC-negative, HPV-negative condition, OPC-negative condition and OPC-negative, HPV-negative condition) and subjects with a second condition (e.g., another of HPV-positive OPC, OPC-negative HPV infection, HPV-negative OPC, HPV-agnostic OPC, OPC-negative, HPV-negative condition, OPC-negative condition and OPC-negative, HPV-negative condition), or to discriminate between OPC-positive subjects with decreased or poor survival prognosis and OPC-positive subjects with increased or good survival prognosis, with at least about 50%, 55% 60%, 65%, 70%, 75%, 80%, 85%, 90%, 95% accuracy or having a C-statistic of at least about 0.50, 0.55, 0.60, 0.65, 0.70, 0.75, 0.80, 0.85, 0.90, 0.95.

In the case of a positive likelihood ratio, a value of 1 indicates that a positive result is equally likely among subjects in both the “first condition” and “second condition” groups; a value greater than 1 indicates that a positive result is more likely in the first condition group; and a value less than 1 indicates that a positive result is more likely in the second condition group. In this context, “first condition” group is meant to refer to a group having one characteristic (e.g., one of the disclosed conditions or prognoses) and “second condition” group lacking the same characteristic. In the case of a negative likelihood ratio, a value of 1 indicates that a negative result is equally likely among subjects in both the “first condition” and “second condition” groups; a value greater than 1 indicates that a negative result is more likely in the “first condition” group; and a value less than 1 indicates that a negative result is more likely in the “second condition” group. In the case of an odds ratio, a value of 1 indicates that a positive result is equally likely among subjects in both the “first condition” and “second condition” groups; a value greater than 1 indicates that a positive result is more likely in the “first condition” group; and a value less than 1 indicates that a positive result is more likely in the “second condition” group. In the case of an AUC ROC value, this is computed by numerical integration of the ROC curve. The range of this value can be 0.5 to 1.0. A value of 0.5 indicates that a classifier (e.g., a miRNA biomarker profile) is no better than a 50% chance to classify unknowns correctly between two groups of interest (e.g., one of the conditions disclosed herein and another of the conditions disclosed herein), while 1.0 indicates the relatively best diagnostic accuracy. In certain embodiments, individual miRNA biomarkers and/or miRNA biomarker panels are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, individual miRNA biomarkers and/or miRNA biomarker panels are selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less.

In certain embodiments, individual miRNA biomarkers and/or miRNA biomarker panels are selected to exhibit an AUC ROC value of greater than 0.5, preferably at least 0.6, more preferably 0.7, still more preferably at least 0.8, even more preferably at least 0.9, and most preferably at least 0.95.

In some cases, multiple thresholds may be determined in so-called “tertile,” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” may be considered “thresholds.” A risk (of a particular diagnosis or prognosis for example) can be assigned based on which “bin” a test subject falls into.

In other embodiments, particular thresholds for the miRNA biomarker(s) measured are not relied upon to determine if the biomarker level(s) obtained from a subject are correlated to a particular diagnosis or prognosis. For example, a temporal change in the miRNA biomarker(s) can be used to rule in or out one or more particular diagnoses and/or prognoses. Alternatively, miRNA biomarker(s) may be correlated to a condition, disease, prognosis, etc., by the presence or absence of one or more miRNA biomarkers in a particular assay format. In the case of miRNA biomarker panels, the detection methods disclosed herein may utilize an evaluation of the entire population or subset of miRNA biomarkers disclosed herein to provide a single result value (e.g., a “panel response” value expressed either as a numeric score or as a percentage risk). In such embodiments, an increase, decrease, or other change (e.g., slope over time) in a certain subset of miRNA biomarkers may be sufficient to indicate a particular condition or future outcome in one patient, while an increase, decrease, or other change in a different subset of miRNA biomarkers may be sufficient to indicate the same or a different condition or outcome in another patient.

In certain embodiments, a panel of miRNA biomarkers is selected to assist in distinguishing a pair of groups (i.e., assist in assessing whether a subject has an increased likelihood of being in one group or the other group of the pair) selected from “HPV-positive OPC”, “OPC-negative HPV infection”, “HPV-negative OPC”, “HPV-agnostic OPC”, “OPC-negative”, “HPV-negative condition”, “OPC-negative condition” and “OPC-negative, HPV-negative condition”, or “low risk” and “high risk” with at least about 70%, 80%, 85%, 90% or 95% sensitivity, suitably in combination with at least about 70% 80%, 85%, 90% or 95% specificity. In some embodiments, both the sensitivity and specificity are at least about 75%, 80%, 85%, 90% or 95%.

The phrases “assessing the likelihood” and “determining the likelihood,” as used herein, refer to methods by which the skilled artisan can predict the presence or absence of a condition (e.g., a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition), or can predict survival in a patient. The skilled artisan will understand that this phrase includes within its scope an increased probability that a condition is present or absent in a patient; that is, that a condition is more likely to be present or absent in a subject. For example, the probability that an individual identified as having a specified condition actually has the condition may be expressed as a “positive predictive value” or “PPV.” Positive predictive value can be calculated as the number of true positives divided by the sum of the true positives and false positives. PPV is determined by the characteristics of the predictive methods disclosed herein as well as the prevalence of the condition in the population analyzed. The statistical algorithms can be selected such that the positive predictive value in a population having a condition prevalence is in the range of 70% to 99% and can be, for example, at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition or prognosis actually does not have that condition or prognosis may be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined as having a significant likelihood of having or not having a specified condition (e.g., HPV-positive OPC, OPC-negative HPV infection, HPV-negative OPC, HPV-agnostic OPC, OPC-negative, HPV-negative condition, OPC-negative condition or OPC-negative, HPV-negative condition) or survival prognosis (e.g., decreased or poor survival prognosis or increased or good survival prognosis). By “significant likelihood” is meant that the subject has a reasonable probability (0.6, 0.7, 0.8, 0.9 or more) of having, or not having, a specified condition or prognosis.

The miRNA biomarker analysis disclosed herein permits the generation of high-density data sets that can be evaluated using informatics approaches. High data density informatics analytical methods are known and software is available to those in the art, e.g., cluster analysis (Pirouette, Informetrix), class prediction (SIMCA-P, Umetrics), principal components analysis of a computationally modeled dataset (SIMCA-P, Umetrics), 2D cluster analysis (GeneLinker Platinum, Improved Outcomes Software), and metabolic pathway analysis (biotech.icmb.utexas.edu). The choice of software packages offers specific tools for questions of interest (Kennedy et al., Solving Data Mining Problems Through Pattern Recognition. Indianapolis: Prentice Hall PTR, 1997; Golub et al., (2999) Science 286:531-7; Eriksson et al., Multi and Megavariate Analysis Principles and Applications: Umetrics, Umea, 2001). In general, any suitable mathematic analyses can be used to evaluate at least one (e.g., 1, 2, 3, 4, 5, 6, 7, 8, 9, 10, etc.) miRNA biomarker in a population disclosed herein with respect to a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition, or to a survival prognosis. For example, methods such as multivariate analysis of variance, multivariate regression, and/or multiple regression can be used to determine relationships between dependent variables (e.g., clinical measures) and independent variables (e.g., levels of miRNA biomarkers). Clustering, including both hierarchical and non-hierarchical methods, as well as non-metric Dimensional Scaling can be used to determine associations or relationships among variables and among changes in those variables.

In addition, principal component analysis is a common way of reducing the dimension of studies, and can be used to interpret the variance-covariance structure of a data set. Principal components may be used in such applications as multiple regression and cluster analysis. Factor analysis is used to describe the covariance by constructing “hidden” variables from the observed variables. Factor analysis may be considered an extension of principal component analysis, where principal component analysis is used as parameter estimation along with the maximum likelihood method. Furthermore, simple hypothesis such as equality of two vectors of means can be tested using Hotelling's T squared statistic.

In some embodiments, the data sets corresponding to miRNA biomarker panels disclosed herein are used to create a diagnostic or predictive rule or model based on the application of a statistical and machine learning algorithm. Such an algorithm uses relationships between a miRNA biomarker panel and a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition, observed in control subjects or typically cohorts of control subjects (sometimes referred to as training data), which provides combined control or reference miRNA biomarker panels for comparison with miRNA biomarker panels of a subject. The data are used to infer relationships that are then used to predict the status of a subject, including the presence or absence of one of the conditions referred to herein.

Practitioners skilled in the art of data analysis recognize that many different forms of inferring relationships in the training data may be used without materially changing the detection methods disclosed herein. The data presented in the Tables, Examples and Figures herein have been used to generate illustrative minimal combinations of miRNA biomarkers (models) that differentiate between one of the disclosed conditions and another of the disclosed conditions using feature selection based on AUC maximization in combination with analytical model classification, including for example classification using one or more of: an additive model; a linear model; a support vector machine; a neural network model; a random forest model; a regression model; a genetic algorithm; an annealing algorithm; a weighted sum; a nearest neighbor model; and a probabilistic model. The miRNA biomarker tables disclosed herein provide illustrative lists of miRNA biomarkers ranked according to their p value. Illustrative models comprising at least 1, at least 2, at least 3, at least 4, at least 5, at least 6, at least 7 miRNA biomarkers were able to develop a classifier or generative algorithm for discriminating between two control groups as defined above with significantly improved positive predictive values compared to conventional methodologies. This algorithm can be advantageously applied to determine presence or probability of one of the conditions or prognoses disclosed herein in a patient, and thus diagnose the patient as having or as likely to have the condition, or prognose the patient as having decreased or poor survival prognosis, or as having increased or good survival prognosis.

In some embodiments, evaluation of miRNA biomarkers includes determining the levels of individual miRNA biomarkers, which correlate with the presence or absence of a condition, as defined above. In certain embodiments, the techniques used for detection of miRNA biomarkers may include internal or external standards to permit quantitative or semi-quantitative determination of those biomarkers, to thereby enable a valid comparison of the level of the miRNA biomarkers in a saliva sample with the corresponding miRNA biomarkers in a reference sample or samples. Such standards can be determined by the skilled practitioner using standard protocols. In specific examples, absolute values for the level or functional activity of individual expression products are determined.

In semi-quantitative methods, a threshold or cut-off value is suitably determined, and is optionally a predetermined value. In particular embodiments, the threshold value is predetermined in the sense that it is fixed, for example, based on previous experience with the assay and/or a population of affected and/or unaffected subjects. Alternatively, the predetermined value can also indicate that the method of arriving at the threshold is predetermined or fixed even if the particular value varies among assays or may even be determined for every assay run.

In some embodiments, the level of a miRNA biomarker is normalized against a housekeeping biomarker. The term “housekeeping biomarker” refers to a biomarker or group of biomarkers (e.g., polynucleotides and/or polypeptides), which are typically found at a constant level in the cell type(s) being analyzed and across the conditions being assessed. In some embodiments, the housekeeping biomarker is a “housekeeping gene.” A “housekeeping gene” may refer herein to a gene or group of genes which encode proteins whose activities are essential for the maintenance of cell function and which are typically found at a constant level in the cell type(s) being analyzed and across the conditions being assessed. In specific embodiments, the housekeeping biomarker is a housekeeping small nuclear RNA, which in illustrative examples is SNORD96A. A representative nucleic acid sequence for SNORD96A is: cctggtgatgacagatggcattgtcagccaatccccaagtgggagtgaggacatgtcctgcaattctgaagg [SEQ ID NO: 15]

There is no intended limitation on the methodology used to normalize the values of the measured biomarkers provided that the same methodology is used for testing a human subject sample as was used to generate the risk categorization table or threshold value. Many methods for data normalization exist and are familiar to those skilled in the art. These include methods such as background subtraction, scaling, MoM analysis, linear transformation, least squares fitting, etc. The goal of normalization is to equate the varying measurement scales for the separate biomarkers such that the resulting values may be combined according to a weighting scale as determined and designed by the user or by the machine learning system and are not influenced by the absolute or relative values of the miRNA biomarker found within nature.

Composite scores may be calculated using standard statistical analysis well known to one of skill in the art wherein the measurements of each miRNA biomarker in the panel are combined, optionally with clinical parameters, to provide a probability value. For example, generalized or multivariate logistic regression analysis may be used to derive a mathematical function with a set of variables corresponding to each miRNA biomarker and optional clinical parameter, which provides a weighting factor for each variable. The weighting factors are derived to optimize the agency of the function to predict the dependent variable, which is the dichotomy of a first condition (e.g., HPV-positive OPC) as compared to a second condition (e.g., OPC-negative HPV infection) disclosed herein. The weighting factors are specific to the particular variable combination (e.g., biomarker panel analyzed). The function can then be applied to the original samples to predict a probability of a disclosed condition. In this way, a retrospective data set may be used to provide weighting factors for a particular panel of salivary miRNA biomarkers, optionally in combination with clinical parameters, which is then used to calculate the probability of a disclosed condition in a patient where the outcome of the condition is unknown or indeterminate prior to screening using the present methods.

Composite scores may be calculated for example using the statistical methodology disclosed in US Publ. No. 2008/013314 for handling and interpreting data from a multiplex assay. In this methodology, the amount of any one biomarker is compared to a predetermined cut-off distinguishing positive from negative for that biomarker as determined from a control population study of patients with a condition (e.g., cancer) and suitably matched normal controls to yield a score for each biomarker based on that comparison; and then combining the scores for each biomarker to obtain a composite score for the biomarker(s) in the sample.

A predetermined cut-off can be based on ROC curves and the score for each biomarker can be calculated based on the specificity of the biomarker. Then, the total score can be compared to a predetermined total score to transform that total score to a qualitative determination of the likelihood or risk of having a condition as disclosed herein.

In certain embodiments, the miRNA biomarkers disclosed herein are measured and those resulting values normalized and then summed to obtain a composite score. In certain aspects, normalizing the measured biomarker values comprises determining the multiple of median (MoM) score. In other aspects, the present method further comprises weighting the normalized values before summing to obtain a composite score. In illustrative examples of this type, the median value of each biomarker is used to normalize all measurements of that specific biomarker, for example, as provided in Kutteh et al. (Obstet. Gynecol. 1994; 84:811-815) and Palomaki et al. (Clin. Chem. Lab. Med 2001; 39:1137-1145). Thus, any measured biomarker level is divided by the median value of a disclosed condition group (e.g., a group selected from HPV-positive OPC, OPC-negative HPV infection, HPV-negative OPC, HPV-agnostic OPC, OPC-negative, HPV-negative condition, OPC-negative condition and OPC-negative, HPV-negative condition), resulting in a MoM value. The MoM values can be combined (namely, summed or added) for each biomarker in the panel resulting in a panel MoM value or aggregate MoM score for each sample.

If desired, a machine learning system may be utilized to determine weighting of the normalized values as well as how to aggregate the values (e.g., determine which miRNA biomarkers are most predictive, and assign a greater weight to these markers).

In specific embodiments, a composite score for determining an indicator used in assessing a likelihood of having HPV-positive OPC in a group consist of HPV-positive OPC and HPV-positive controls is determined by a statistical model based on Generalized Regression where Logist(x)=1/(1+Exp(−x)) outlines the probability of having HPV-positive OPC which increases from 0 to 1. A probability above the cut-off 0.6383 indicates HPV-positive OPC (sensitivity−89.1% (77.0%, 95.3%), specificity−80.0% (54.8%, 93.0%)). Logist(x) is calculated using the following formula: [Logist (−3.037301948-0.358714177*ΔCT·Hsa-miR-194-5p−0.044825031*ΔCT·Hsa-miR-501-3p−0.504468334*ΔCT·Hsa-miR-548K)] where ΔCT is the relative expression of the target miRNA compared to the reference (SNORD96A).

In other specific embodiments, a composite score for determining an indicator used in assessing a likelihood of having HPV-negative OPC in a group consist of HPV-negative OPC and HPV-negative controls is determined by a statistical model based on Generalized Regression where Logist(x)=1/(1+Exp(−x)) outlines the probability of having HPV-negative OPC which increases from 0 to 1. A probability above the cut-off 0.6383 indicates HPV-negative OPC (sensitivity −84.6% (57.8%, 95.7%), specificity −93.5% (82.5%, 97.8%). Logist(x) is calculated using the following formula: [Logist (4.2853649881+0.087895243*ΔCT·Hsa-miR-215-3p−0.230394738*ΔCT·Hsa-miR-194-5p+0.0909199175*ΔCT·Hsa-miR-449a+0.6178016271*ΔCT·Hsa-miR-07-5p+0.5975870736*ΔCT·Hsa-miR-501-3p−0.001857522*ΔCT·Hsa-miR-1290-0.367553405*ΔCT·Hsa-miR-548K)] where ΔCT is the relative expression of the target miRNA compared to the reference (SNORD96A).

In still other specific embodiments, a composite score for determining an indicator used in assessing a likelihood of having OPC (either HPV+ or HPV−) in a group consist of OPC (either HPV+ or HPV−) and controls (either HPV+ and HPV−) is determined by a statistical model based on Generalized Regression where Logist(x)=1/(1+Exp(−x)) outlines the probability of having OPC (either HPV+ or HPV−) which increases from 0 to 1. A probability above the cut-off 0.5 indicates OPC (either HPV+ or HPV−) (sensitivity −74.6% (62.2%, 83.9%), specificity −66.1% (53.7%, 76.7%)). Logist(x) is determined using the following formula based on Generalized Regression: [Logist (−0.270402942-0.10690701*ΔCT·Hsa-miR-3614-5p−0.253253038*ΔCT·Hsa-miR-501-3p−126695534*ΔCT·Hsa-miR-1290-0.100609157*ΔCT·Hsa-miR-1246)] where ΔCT is the relative expression of the target miRNA compared to the reference (SNORD96A).

In some embodiments, composite scores include one or more clinical parameters or signs of the patient. Representative clinical parameters or signs include age, ethnicity, gender, diastolic blood pressure and systolic blood pressure, family history, height, weight, waist and hip circumference, body-mass index, resting heart rate, β-cell function, macrovascular function, microvascular function, atherogenic index, blood pressure, low-density lipoprotein/high-density lipoprotein ratio, intima-media thickness, soreness (e.g., throat, mouth or neck) lump presence (e.g., throat, mouth or neck), swallow function, mouth function, tongue function, weight loss, pain (e.g., ear), tissue discoloration (e.g., the tongue or lining of the mouth), and hemoptysis.

In certain embodiments, the detection methods utilize a risk categorization table to generate a risk score for a patient based on a composite score by comparing the composite score with a reference set derived from a cohort of patients with one of the conditions disclosed herein. The detection methods may further comprise quantifying the increased risk for the presence of the condition in the subject as a risk score, wherein the composite score (combined obtained biomarker value and optionally obtained clinical parameter values) is matched to a risk category of a grouping of stratified subject populations wherein each risk category comprises a multiplier (or percentage) indicating an increased likelihood of having the condition correlated to a range of composite scores. This quantification is based on the pre-determined grouping of a stratified cohort of subjects. In some embodiments, the grouping of a stratified population of subjects, or stratification of a disease cohort, is in the form of a risk categorization table. The selection of the disease cohort, the cohort of subjects that share disclosed condition risk factors, are well understood by those skilled in the art of cancer research. However, the skilled person would also recognize that the resulting stratification, may be more multidimensional and take into account further environmental, occupational, genetic, or biological factors (e.g., epidemiological factors).

After quantifying the increased risk for presence of a condition (e.g., HPV-positive OPC, OPC-negative HPV infection, HPV-negative OPC, HPV-agnostic OPC, OPC-negative, HPV-negative condition, OPC-negative condition or OPC-negative, HPV-negative condition) or survival prognosis (e.g., decreased or poor survival prognosis, or increased or good survival prognosis) in the form of a risk score, this score may be provided in a form amenable to understanding by a physician. In certain embodiments, the risk score is provided in a report. In certain aspects, the report may comprise one or more of the following: patient information, a risk categorization table, a risk score relative to a cohort population, one or more biomarker test scores, a biomarker composite score, a master composite score, identification of the risk category for the patient, an explanation of the risk categorization table, and the resulting test score, a list of biomarkers tested, a description of the disease cohort, environmental and/or occupational factors, cohort size, biomarker velocity, genetic mutations, family history, margin of error, and so on.

### 3. Kits

All the essential reagents required for detecting and quantifying the miRNA biomarkers disclosed herein may be assembled together in a kit. In some embodiments, the kit comprises a reagent that permits quantification of at least one miRNA biomarker or each miRNA biomarker of a biomarker panel disclosed herein. In the context of the present disclosure, “kit” is understood to mean a product containing the different reagents necessary for carrying out the methods of the disclosure packed so as to allow their transport and storage. Additionally, the kits of the present disclosure can contain instructions for the simultaneous, sequential or separate use of the different components contained in the kit. The instructions can be in the form of printed material or in the form of an electronic support capable of storing instructions such that they can be read by a subject, such as electronic storage media (magnetic disks, tapes and the like), optical media (CD-ROM, DVD) and the like. Alternatively or in addition, the media can contain internet addresses that provide the instructions. The kits may contain software for interpreting assay data to determine the likelihood of the presence or absence of a condition disclosed herein, or likelihood of an OPC-positive patient having a decreased or poor survival prognosis, or an increased or good survival prognosis. In some embodiments, the kits may provide a means to access a machine learning system provided, for example, as a software as a service (SaaS) deployment.

Reagents that allow quantification of a miRNA biomarker include compounds or materials, or sets of compounds or materials, which allow quantification of the miRNA biomarker. In specific embodiments, the compounds, materials or sets of compounds or materials permit determining the expression level of a gene (e.g., a miRNA gene disclosed herein) include without limitation the extraction of RNA material, the determination of the level of a corresponding RNA, etc., primers for the synthesis of a corresponding cDNA, primers for amplification of DNA, and/or probes capable of specifically hybridizing with the RNAs (or the corresponding cDNAs) encoded by the genes, TaqMan™ probes, etc.

Kit reagents can be in liquid form or can be lyophilized. Suitable containers for the reagents include, for example, bottles, vials, syringes, and test tubes. Containers can be formed from a variety of materials, including glass or plastic. The kit can also comprise a package insert containing written instructions for methods of diagnosing a condition disclosed herein or prognosis patient survival.

The kits may also optionally include appropriate reagents for detection of labels, positive and negative controls, washing solutions, blotting membranes, microtiter plates, dilution buffers and the like. For example, a nucleic acid-based detection kit may include (i) a miRNA biomarker (which may be used as a positive control), (ii) a primer or probe that specifically hybridizes to a miRNA biomarker. Also included may be enzymes suitable for amplifying nucleic acids including various polymerases (reverse transcriptase, Taq polymerase, Sequenase™, DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. The kit can also feature various devices (e.g., one or more) and reagents (e.g., one or more) for performing one of the assays described herein; and/or printed instructions for using the kit to quantify the expression of a miRNA biomarker gene and/or carry out an indicator-determining method, as broadly described above and elsewhere herein.

The reagents described herein, which may be optionally associated with detectable labels, can be presented in the format of a microfluidics card, a reaction vessel, a microarray or a kit adapted for use with the assays described in the examples or below, e.g., RT-PCR or Q PCR techniques described herein.

The reagents also have utility in compositions for detecting and quantifying the miRNA biomarkers of the present disclosure. For example, a reverse transcriptase may be used to reverse transcribe RNA transcripts, including mRNA, in a nucleic acid sample, to produce reverse transcribed transcripts, including reverse transcribed mRNA (also referred to as “cDNA”). In specific embodiments, the reverse transcribed mRNA is whole cell reverse transcribed mRNA (also referred to herein as “whole cell cDNA”). The nucleic acid sample is suitably derived from a salivary sample as disclosed for example herein.

The reagents are suitably used to quantify the reverse transcribed transcripts. For example, oligonucleotide primers that hybridize to the reverse transcribed transcript can be used to amplify at least a portion of the reverse transcribed transcript via a suitable nucleic acid amplification technique, e.g., RT-PCR or qPCR techniques described herein. Alternatively, oligonucleotide probes may be used to hybridize to the reverse transcribed transcript for the quantification, using a nucleic acid hybridization analysis technique (e.g., microarray analysis). Thus, in some embodiments, a respective oligonucleotide primer or probe is hybridized to a complementary nucleic acid sequence of a reverse transcribed miRNA transcript in the compositions of the present disclosure. The compositions typically comprise labeled reagents for detecting and/or quantifying the reverse transcribed transcripts. Representative reagents of this type include labeled oligonucleotide primers or probes that hybridize to RNA transcripts or reverse transcribed RNA, labeled RNA, labeled reverse transcribed RNA as well as labeled oligonucleotide linkers or tags (e.g., a labeled RNA or DNA linker or tag) for labeling (e.g., end labeling such as 3′ end labeling) RNA or reverse transcribed RNA. The primers, probes, RNA or reverse transcribed RNA (i.e., cDNA) (whether labeled or non-labeled) may be immobilized or free in solution. Representative reagents of this type include labeled oligonucleotide primers or probes that hybridize to reverse transcribed and transcripts as well as labeled reverse transcribed transcripts. The label can be any reporter molecule as known in the art, illustrative examples of which are described above and elsewhere herein.

The kits disclosed herein also encompass non-reverse transcribed RNA embodiments in which cDNA is not made and the miRNA transcripts are directly the subject of the analysis. Thus, in other embodiments, reagents are suitably used to quantify miRNA transcripts directly. For example, oligonucleotide probes can be used to hybridize to transcripts for quantification of miRNA biomarkers of the present disclosure, using a nucleic acid hybridization analysis technique (e.g., microarray analysis). Thus, in some embodiments, a respective oligonucleotide probe is hybridized to a complementary nucleic acid sequence of a miRNA biomarker transcript in the disclosed compositions. In illustrative examples of this type, the compositions may comprise labeled reagents that hybridize to transcripts for detecting and/or quantifying the transcripts. Representative reagents of this type include labeled oligonucleotide probes that hybridize to transcripts as well as labeled transcripts. The primers or probes may be immobilized or free in solution.

### 4. Treatment Embodiments

Also disclosed herein are methods for treating or managing the development or progression of OPC in a human subject, suitably one with at least one clinical sign of OPC. A subject positively identified as having OPC may be exposed to a treatment regimen, illustrative examples of which include: surgery, radiotherapy, chemotherapy, hormone ablation therapy, pro-apoptosis therapy, cryotherapy and immunotherapy.

Radiotherapies include radiation and waves that induce DNA damage for example, γ-irradiation, X-rays, UV irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. Non-limiting examples of radiotherapies include conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine.

Chemotherapeutic agents may be cytostatic or cytotoxic. Non-limiting examples of chemotherapeutic agents for use in accordance with the treatment methods of the present disclosure include any one or more of those in the following categories:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyridines like 5-fluorouracil and tegafur, raltitrexed, methotrexate, cytosine arabinoside and hydroxyurea; anti-tumor antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and docetaxel; and topoisomerase inhibitors (for example epipodophyllotoxins like etoposide and teniposide, amsacrine, topotecan and camptothecin);
(ii) cytostatic agents such as antiestrogens (for example tamoxifen, toremifene, raloxifene, droloxifene and idoxifene), oestrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorozole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazoli-n-4-amine (CI 1033)), for example inhibitors of the platelet-derived growth factor family and for example inhibitors of the hepatocyte growth factor family;
(v) anti-angiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO0/40529, WO 00/41669, WO01/92224, WO02/04434 and WO02/08213; [0226](vii) antisense therapies, for example those which are directed to the targets listed above, such as ISIS 2503, an anti-ras antisense; and
(viii) gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

(ix) immunotherapy approaches, including for example immune checkpoint such as: those that target CTLA-4 and thus block or inhibit the interaction between CTLA-4 and CD80/CD86 (i.e. CTLA-4 inhibitors, such as ipilimumab or tremelimumab); those that target PD-1 and thus block or inhibit the interaction between PD-1 and PD-L1 (i.e. PD-1 inhibitors, representative examples of which include pembrolizumab, pidilizumab, nivolumab, REGN2810, CT-001, AMP-224, BMS-936558, MK-3475, MEDIO680 and PDR001); and those that target PD-L1 and thus block or inhibit the interaction between PD-1 and PD-L1 (i.e. PD-L1 inhibitors such as atezolizumab, durvalumab, avelumab, BMS-936559 and MEDI4736). Alternatively, or in addition, ex vivo and in vivo approaches may be used to increase the immunogenicity of patient tumor cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumor cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

Typically, cancer therapeutic agents are administered in pharmaceutical (or veterinary) compositions together with a pharmaceutically acceptable carrier and in an effective amount to achieve their intended purpose. The dose of active compounds administered to a subject should be sufficient to achieve a beneficial response in the subject over time, such as a reduction in tumor burden and the like. The quantity of the pharmaceutically active compounds(s) to be administered may depend on the subject to be treated inclusive of the age, sex, weight and general health condition thereof. In this regard, precise amounts of the active compound(s) for administration will depend on the judgment of the practitioner. In determining the effective amount of the active compound(s) to be administered in the treatment of OPC, the medical practitioner may evaluate one or more clinical signs associated with the presence of OPC, including the severity of clinical signs. In any event, those of skill in the art may readily determine suitable dosages of the therapeutic agents and suitable treatment regimens without undue experimentation.

The OPC therapy may be administered in concert with an adjunctive cancer therapy, representative examples of which include agents to reduce pain, hair loss, vomiting, immune suppression, nausea, diarrhea, rash, sensory disturbance, anemia, fatigue, stomatitis, or hand foot syndrome.

In embodiments relating to an indication that an OPC patient has decreased or poor survival prognosis, the patient may be administered an alternative cancer therapy including combination therapy, or with an increased dosage of a cancer therapy agent, or may be placed into palliative care.

### 5. Device Embodiments

Also contemplated herein are embodiments in which the indicator-determining method of the invention is implemented using one or more processing devices. In representative embodiments of this type, the method that is implemented by the processing device(s) determines an indicator used in assessing a likelihood of a human subject having a presence or absence a condition disclosed herein (e.g., HPV-positive OPC, OPC-negative HPV infection, HPV-negative OPC, HPV-agnostic OPC, OPC-negative, HPV-negative condition, OPC-negative condition or OPC-negative, HPV-negative condition) or has a survival prognosis (e.g., decreased or poor survival prognosis or increased or good survival prognosis), wherein the method comprises: (1) determining a biomarker value for at least one miRNA biomarker (e.g., 1, 2, 3, 4, 5, 6, 7, or more biomarkers) disclosed herein in a saliva sample obtained from the subject; (2) determining the indicator using the biomarker value(s); (3) retrieving previously determined indicator references from a database, the indicator references being determined based on indicators determined from a reference population consisting of individuals diagnosed with the condition or individual having the survival prognosis; (4) comparing the indicator to the indicator references to thereby determine a probability indicative of the subject having or not having the condition, or having or not having the survival prognosis; and (5) generating a representation of the probability, the representation being displayed to a user to allow the user to assess the likelihood of the subject having the condition or survival prognosis.

In specific embodiments, an apparatus is provided for determining the likelihood of a human subject having a condition disclosed herein (e.g., HPV-positive OPC, OPC-negative HPV infection, HPV-negative OPC, HPV-agnostic OPC, OPC-negative, HPV-negative condition, OPC-negative condition or OPC-negative, HPV-negative condition) or having a survival prognosis (e.g., decreased or poor survival prognosis or increased or good survival prognosis). The apparatus typically includes at least one electronic processing device that:
determines a biomarker value for at least one miRNA biomarker (e.g., 1, 2, 3, 4, 5, 6, 7, 8, 9, 10, 11, 12 or more biomarkers) disclosed herein in a saliva sample obtained from the subject; and
determines the indicator using the biomarker value(s).

The apparatus may further include any one or more of:
(A) a sampling device that obtains a sample taken from a subject, the sample including at least one miRNA biomarker (e.g., 1, 2, 3, 4, 5, 6, 7, or more biomarkers) disclosed herein;
(B) a measuring device that quantifies for each of the miRNA biomarkers a corresponding a biomarker value;
(C) at least one processing device that:
   (i) receives the biomarker value(s) from the measuring device;
   (ii) determines an indicator that is indicative of the presence or absence of the condition or survival prognosis using the biomarker values optionally in combination with one or more clinical parameters or signs of the subject;
   (iii) compares the indicator to at least one indicator reference;
   (iv) determines a likelihood of the subject having or not having condition or survival prognosis using the results of the comparison; and
   (v) generates a representation of the indicator and the likelihood for display to a user.

In some embodiments, the apparatus comprises a processor configured to execute computer readable media instructions (e.g., a computer program or software application, e.g., a machine learning system, to receive the biomarker values from the evaluation of miRNA biomarkers in a sample and, in combination with other risk factors (e.g., medical history of the patient, publically available sources of information pertaining to a risk of OPC) may determine a master composite score and compare it to a grouping of stratified cohort population comprising multiple risk categories (e.g., a risk categorization table) and provide a risk score. Methods and techniques for determining a master composite score and a risk score are known in the art.

The apparatus can take any of a variety of forms, for example, a handheld device, a tablet, or any other type of computer or electronic device. The apparatus may also comprise a processor configured to execute instructions (e.g., a computer software product, an application for a handheld device, a handheld device configured to perform the method, a world-wide-web (WWW) page or other cloud or network accessible location, or any computing device. In other embodiments, the apparatus may include a handheld device, a tablet, or any other type of computer or electronic device for accessing a machine learning system provided as a software as a service (SaaS) deployment. Accordingly, the correlation may be displayed as a graphical representation, which, in some embodiments, is stored in a database or memory, such as a random access memory, read-only memory, disk, virtual memory, etc. Other suitable representations, or exemplifications known in the art may also be used.

The apparatus may further comprise a storage means for storing the correlation, an input means, and a display means for displaying the status of the subject in terms of the particular medical condition (e.g., HPV-positive OPC, OPC-negative HPV infection, HPV-negative OPC, HPV-agnostic OPC, OPC-negative, HPV-negative condition, OPC-negative condition or OPC-negative, HPV-negative condition). The storage means can be, for example, random access memory, read-only memory, a cache, a buffer, a disk, virtual memory, or a database. The input means can be, for example, a keypad, a keyboard, stored data, a touch screen, a voice-activated system, a downloadable program, downloadable data, a digital interface, a hand-held device, or an infrared signal device. The display means can be, for example, a computer monitor, a cathode ray tube (CRT), a digital screen, a light-emitting diode (LED), a liquid crystal display (LCD), an X-ray, a compressed digitized image, a video image, or a hand-held device. The apparatus can further comprise or communicate with a database, wherein the database stores the correlation of factors and is accessible to the user.

In certain embodiments, the apparatus is a computing device, for example, in the form of a computer or hand-held device that includes a processing unit, memory, and storage. The computing device can include, or have access to a computing environment that comprises a variety of computer-readable media, such as volatile memory and non-volatile memory, removable storage and/or non-removable storage. Computer storage includes, for example, RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or other medium known in the art to be capable of storing computer-readable instructions. The computing device can also include or have access to a computing environment that comprises input, output, and/or a communication connection. The input can be one or several devices, such as a keyboard, mouse, touch screen, or stylus. The output can also be one or several devices, such as a video display, a printer, an audio output device, a touch stimulation output device, or a screen reading output device. If desired, the computing device can be configured to operate in a networked environment using a communication connection to connect to one or more remote computers. The communication connection can be, for example, a Local Area Network (LAN), a Wide Area Network (WAN) or other networks and can operate over the cloud, a wired network, wireless radio frequency network, and/or an infrared network.

### Representative Embodiments

1. A method for determining an indicator used in assessing a likelihood that human papillomavirus (HPV)-positive oropharyngeal cancer (OPC) or OPC-negative HPV infection is present or absent in a human subject, the method comprising, consisting or consisting essentially of:
(1) determining a biomarker value for at least one miRNA biomarker (e.g., 1, 2 or 3 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-501-3p and Hsa-miR-548K; and
(2) determining the indicator using the biomarker value(s).

2. A method for determining an indicator used in assessing a likelihood that HPV-positive OPC or OPC-negative HPV infection is present or absent in a human subject, the method comprising, consisting or consisting essentially of:
(1) determining a biomarker value for at least one miRNA biomarker (e.g., 1, 2, 3, 4, 5, 6, 7, 8 or 9 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K; and
(2) determining the indicator using the biomarker value(s).

3. A method for determining an indicator used in assessing a likelihood that HPV-negative OPC or an OPC-negative, HPV-negative condition is present or absent in a human subject, the method comprising, consisting or consisting essentially of:
(1) determining a biomarker value for at least one miRNA biomarker (e.g., 1, 2, 3, 4, 5, 6 or 7 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K; and
(2) determining the indicator using the biomarker value(s).

4. A method for determining an indicator used in assessing a likelihood that HPV-negative OPC or an OPC-negative, HPV-negative condition is present or absent in a human subject, the method comprising, consisting or consisting essentially of:
(1) determining a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-449a, Hsa-miR-07-5p, Hsa-miR-501-3p, Hsa-miR-1290 and Hsa-miR-548K; and
(2) determining the indicator using the biomarker value(s).

5. A method for determining an indicator used in assessing a likelihood that HPV-agnostic OPC or an OPC-negative condition is present or absent in a human subject, the method comprising, consisting or consisting essentially of:
(1) determining a biomarker value for at least one miRNA biomarker (e.g., 1, 2, 3 or 4 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-3614-5p, Hsa-miR-501-3p, Hsa-miR-1290 and Hsa-miR-1246; and
(2) determining the indicator using the biomarker value(s).

6. A method for determining an indicator used in assessing a likelihood that HPV-agnostic OPC or an OPC-negative condition (e.g., HPV-positive, OPC-negative condition, HPV-negative, OPC-negative condition) is present or absent in a human subject, the method comprising, consisting or consisting essentially of:
(1) determining a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-449a, Hsa-miR-3614-5p, Hsa-miR-07-5p, Hsa-miR-501-3p, Hsa-miR-1290 and Hsa-miR-548K; and
(2) determining the indicator using the biomarker value(s).

7. The method of any one of embodiments 1 to 6, wherein biomarker values are determined for at least two miRNA biomarkers.

8. The method of any one of embodiments 1 to 6, wherein biomarker values are determined for at least three miRNA biomarkers.

9. The method of any one of embodiments 2 to 6, wherein biomarker values are determined for at least four biomarkers.

10. The method of any one of embodiments 2 to 4 and 6, wherein biomarker values are determined for at least five biomarkers.

11. The method of any one of embodiments 2 to 4 and 6, wherein biomarker values are determined for at least six biomarkers.

12. The method of embodiment 2 or embodiment 3, wherein biomarker values are determined for at least seven biomarkers.

13. The method of embodiment 2, wherein biomarker values are determined for at least eight biomarkers.

14. The method of embodiment 2, wherein biomarker values are determined for at least nine biomarkers.

15. The method of embodiment 1, wherein a biomarker value is obtained for each of Hsa-miR-194-5p, Hsa-miR-501-3p and Hsa-miR-548K.

16. The method of embodiment 2, wherein a biomarker value is obtained for each of Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K.

17. The method of embodiment 3, wherein a biomarker value is obtained for each of Hsa-miR-215-3p, Hsa-miR-194-5p, Hsa-miR-449a, Hsa-miR-07-5p, Hsa-miR-501-3p, Hsa-miR-1290 and Hsa-miR-548K.

18. The method of embodiment 4, wherein a biomarker value is obtained for each of Hsa-miR-194-5p, Hsa-miR-449a, Hsa-miR-07-5p, Hsa-miR-501-3p, Hsa-miR-1290 and Hsa-miR-548K.

19. The method of embodiment 5, wherein a biomarker value is obtained for each of Hsa-miR-3614-5p, Hsa-miR-501-3p, Hsa-miR-1290 and Hsa-miR-1246.

20. The method of embodiment 6, wherein a biomarker value is obtained for each of Hsa-miR-449a, Hsa-miR-3614-5p, Hsa-miR-07-5p, Hsa-miR-501-3p, Hsa-miR-1290 and Hsa-miR-548K.

21. The method of any one of embodiments 1 to 20, wherein the subject has at least one clinical sign of OPC.

22. The method of embodiment 21, wherein the at least one clinical sign is selected from sore throat that does not go away, a lump in the throat, mouth or neck, trouble swallowing, trouble opening the mouth fully, trouble moving the tongue, weight loss for no reason, ear pain, a change in voice, a white patch on the tongue or lining of the mouth that does not go away, and coughing up blood).

23. The method of any one of embodiments 1 to 22, further comprising applying a function to biomarker values to yield at least one functionalized biomarker value and determining the indicator using the at least one functionalized biomarker value.

24. The method of embodiment 23, wherein the function includes at least one of: (a) multiplying biomarker values; (b) dividing biomarker values; (c) adding biomarker values; (d) subtracting biomarker values; (e) a weighted sum of biomarker values; (f) a log sum of biomarker values; (g) a geometric mean of biomarker values; (h) a sigmoidal function of biomarker values; and (i) normalization of biomarker values.

25. The method of any one of embodiments 1 to 24, further comprising combining the biomarker values to provide a composite score and determining the indicator using the composite score.

26. The method of embodiment 25, wherein the biomarker values are combined by adding, multiplying, subtracting, and/or dividing biomarker values.

27. The method of any one of embodiments 1 to 26, further comprising analyzing the biomarker value(s) or composite score with reference to one or more reference biomarker values, value ranges or cut-off values, or reference composite scores, composite score ranges or composite score cut-offs, to determine the indicator.

28. The method of embodiment 27, wherein a respective reference biomarker value, value range or cut-off value, or reference composite score, composite score range or composite score cut-off is a biomarker value, value range or cut-off value, or reference composite score, composite score range or composite score cut-off corresponding to a control subject or control population of subjects.

29. The method of embodiment 28, wherein the condition of the control subject or control population of subjects is the condition analyzed by the indicator.

30. The method of embodiment 28, wherein the condition of the control subject or control population of subjects is a different condition than the condition analyzed by the indicator.

31. The method of embodiment 30, wherein the condition analyzed by the indicator represents a first condition assessed by the method of any one of claims 1 to 27, and the condition of the control subject or control population of subjects represents a second condition assessed by said method.

32. The method of any one of embodiments 27 to 31, wherein the indicator indicates a likelihood of a presence of a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition, if the biomarker value(s) or composite score is indicative of the level of the biomarker(s) in the sample that correlates with an increased likelihood of a presence of the condition relative to a predetermined reference biomarker value, value range or cut-off value, or to a predetermined reference composite score, composite score range or composite score cut-off.

33. A method for determining an indicator used in assessing a likelihood of a human subject with OPC having a survival prognosis selected from a decreased or poor survival prognosis and an increased or good survival prognosis, the method comprising, consisting or consisting essentially of:
(1) determining a biomarker value for Hsa-miR-07-5p in a saliva sample obtained from the subject, wherein the biomarker value is indicative of a level of Hsa-miR-07-5p in the sample; and
(2) determining the indicator using the biomarker value.

34. The method of embodiment 33, further comprising analyzing the biomarker value with reference to a reference biomarker value, value range or cut-off value, to determine the indicator.

35. The method of embodiment 34, wherein the indicator indicates a likelihood of a decreased or poor survival prognosis if the biomarker value for Hsa-miR-07-5p is indicative of a level of Hsa-miR-07-5p in the sample that correlates with an increased likelihood of a decreased or poor survival prognosis relative to a predetermined reference biomarker value, value range or cut-off value.

36. The method of embodiment 34, wherein the indicator indicates a likelihood of an increased or good survival prognosis if the biomarker value for Hsa-miR-07-5p is indicative of a level of Hsa-miR-07-5p in the sample that correlates with an increased likelihood of an increased or good survival prognosis relative to a predetermined reference biomarker value, value range or cut-off value.

37. The method of any one of embodiments 1 to 36, wherein individual biomarker values represent a measured amount or concentration of a corresponding miRNA biomarker in the sample.

38. The method of any one of embodiments 1 to 36, wherein individual biomarker values are a logarithmic representation of a measured amount or concentration of a corresponding miRNA biomarker in the sample.

39. An apparatus for determining an indicator used in assessing a likelihood that HPV-positive OPC or OPC-negative HPV infection is present or absent in a human subject, the apparatus comprising at least one electronic processing device that:
determines a biomarker value for at least one miRNA biomarker (e.g., 1, 2 or 3 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-501-3p and Hsa-miR-548K; and
determines the indicator using the derived biomarker value(s).

40. An apparatus for determining an indicator used in assessing a likelihood that HPV-positive OPC or an OPC-negative, HPV-positive condition is present or absent in a human subject, the apparatus comprising at least one electronic processing device that:
determines a biomarker value for at least one miRNA biomarker (e.g., 1, 2, 3, 4, 5, 6, 7, 8 or 9 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K; and
determines the indicator using the derived biomarker value(s).

41. An apparatus for determining an indicator used in assessing a likelihood that HPV-negative OPC or an OPC-negative, HPV-negative condition is present or absent in a human subject, the apparatus comprising at least one electronic processing device that:
determines a biomarker value for at least one miRNA biomarker (e.g., 1, 2, 3, 4, 5, 6 or 7 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K; and
determines the indicator using the derived biomarker value(s).

42. An apparatus for determining an indicator used in assessing a likelihood that HPV-negative OPC or an OPC-negative, HPV-negative condition is present or absent in a human subject, the apparatus comprising at least one electronic processing device that:
determines a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-449a, Hsa-miR-07-5p, Hsa-miR-501-3p, Hsa-miR-1290 and Hsa-miR-548K; and
determines the indicator using the derived biomarker value(s).

43. An apparatus for determining an indicator used in assessing a likelihood that HPV-agnostic OPC or an OPC-negative condition is present or absent in a human subject, the apparatus comprising at least one electronic processing device that:
determines a biomarker value for at least one miRNA biomarker (e.g., 1, 2, 3 or 4 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-3614-5p, Hsa-miR-501-3p, Hsa-miR-1290 and Hsa-miR-1246; and
determines the indicator using the derived biomarker value(s).

44. An apparatus for determining an indicator used in assessing a likelihood that HPV-agnostic OPC or an OPC-negative condition (e.g., HPV-positive, OPC-negative condition, HPV-negative, OPC-negative condition) is present or absent in a human subject, the apparatus comprising at least one electronic processing device that:
determines a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-449a, Hsa-miR-3614-5p, Hsa-miR-07-5p, Hsa-miR-501-3p, Hsa-miR-1290 and Hsa-miR-548K; and
determines the indicator using the derived biomarker value(s).

45. An apparatus for determining an indicator used in assessing a likelihood of a human subject with OPC having a survival prognosis selected from a decreased or poor survival prognosis and an increased or good survival prognosis, the apparatus comprising at least one electronic processing device that:
determines a biomarker value for Hsa-miR-07-5p in a saliva sample obtained from the subject, wherein the biomarker value is indicative of a level of Hsa-miR-07-5p in the sample; and
determines the indicator using the derived biomarker value.

46. A composition comprising a mixture of a DNA polymerase, saliva cDNA from a human subject, suitably with at least one clinical sign of OPC, wherein the saliva cDNA comprises at least one cDNA biomarker (e.g., 1, 2 or 3 cDNA biomarkers) selected from Hsa-miR-194-5p cDNA, Hsa-miR-501-3p cDNA and Hsa-miR-548K cDNA, and wherein the composition further comprises for a respective cDNA biomarker at least one oligonucleotide primer or probe that hybridizes to the cDNA biomarker.

47. A composition comprising a mixture of a DNA polymerase, saliva cDNA from a human subject, suitably with at least one clinical sign of OPC, wherein the saliva cDNA comprises at least one cDNA biomarker (e.g., 1, 2, 3, 4, 5, 6, 7, 8 or 9 cDNA biomarkers) selected from Hsa-miR-194-5p cDNA, Hsa-miR-449a cDNA, Hsa-miR-3614-5p cDNA, Hsa-miR-07-5p cDNA, Hsa-miR-3529-3p cDNA, Hsa-miR-99A-3p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

48. A composition comprising a mixture of a DNA polymerase, saliva cDNA from a human subject, suitably with at least one clinical sign of OPC, wherein the saliva cDNA comprises at least one cDNA biomarker (e.g., 1, 2, 3, 4, 5, 6 or 7 cDNA biomarkers) selected from Hsa-miR-215-3p cDNA, Hsa-miR-194-5p cDNA, Hsa-miR-449a cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

49. A composition comprising a mixture of a DNA polymerase, saliva cDNA from a human subject, suitably with at least one clinical sign of OPC, wherein the saliva cDNA comprises at least one cDNA biomarker (e.g., 1, 2, 3, 4, 5 or 6 cDNA biomarkers) selected from Hsa-miR-194-5p cDNA, Hsa-miR-449a cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

50. A composition comprising a mixture of a DNA polymerase, saliva cDNA from a human subject, suitably with at least one clinical sign of OPC, wherein the saliva cDNA comprises at least one cDNA biomarker (e.g., 1, 2, 3 or 4 miRNA biomarkers) selected from Hsa-miR-3614-5p cDNA, Hsa-miR-501-3p cDNA, Hsa-miR-1290 cDNA and Hsa-miR-1246 cDNA, and wherein the composition further comprises for a respective cDNA biomarker at least one oligonucleotide primer or probe that hybridizes to the cDNA biomarker.

51. A composition comprising a mixture of a DNA polymerase, saliva cDNA from a human subject, suitably with at least one clinical sign of OPC, wherein the saliva cDNA comprises at least one cDNA biomarker (e.g., 1, 2, 3, 4, 5 or 6 cDNA biomarkers) selected from Hsa-miR-449a cDNA, Hsa-miR-3614-5p cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

52. A composition comprising a mixture of a DNA polymerase, saliva cDNA from a human subject, suitably with at least one clinical sign of OPC, wherein the saliva cDNA comprises a cDNA biomarker corresponding to Hsa-miR-07-5p, and at least one oligonucleotide primer or probe that hybridizes to the cDNA biomarker.

53. The composition of any one of embodiments 46 to 52, wherein the composition comprises for respective cDNA biomarkers two oligonucleotide primers that hybridize to opposite complementary strands of a corresponding cDNA biomarker.

54. The composition of any one of embodiments 46 to 53, wherein the composition comprises for respective cDNA biomarkers an oligonucleotide probe that hybridizes to a corresponding cDNA or a polynucleotide corresponding to the cDNA biomarker (e.g., a polynucleotide product resulting from nucleic acid amplification of the cDNA biomarker).

55. The composition of embodiment 54, wherein the oligonucleotide probe comprises a heterologous reporter molecule.

56. The composition of embodiment 55, wherein the reporter molecule comprises a fluorescent label.

57. The composition of any one of embodiments 46 to 56, wherein the oligonucleotide probe is a real-time polymerase chain reaction probe.

58. The composition of any one of embodiments 46 to 57, wherein the composition comprises for each of at least 1, 2, 3, 4, 5, 6, 7, 8 or 9 cDNA biomarkers at least one oligonucleotide primer and/or probe that hybridizes to the cDNA biomarker.

59. The composition of any one of embodiments 46 to 57, wherein the composition comprises for each of up to 1, 2, 3, 4, 5, 6, 7, 8 or 9 cDNA biomarkers at least one oligonucleotide primer and/or probe that hybridizes to the cDNA biomarker.

60. The composition of any one of embodiments 46 to 51 and 53 to 59, wherein individual cDNA biomarkers and their corresponding oligonucleotide primer(s) and/or probe(s) are present in separate reaction vessels.

61. The composition of any one of embodiments 46 to 51 and 53 to 60, wherein two or more (e.g., 2, 3, 4, 5, 6, 7, 8 or 9) cDNA biomarkers and their corresponding oligonucleotide primer(s) and/or probe(s) are present in the same reaction vessel.

62. The composition of any one of embodiments 46 to 61, wherein the DNA polymerase is a thermostable DNA polymerase.

63. A device for nucleic acid amplification of saliva cDNA, the device comprising a plurality of reaction vessels, individual reaction vessels comprising the composition of any one of embodiments 46 to 62.

64. The device of embodiment 63, consisting of 1 to 12, 1 to 10, 1 to 8, 1 to 6 or 1 to 4 reaction vessels (and all integer reaction vessels in between).

65. The device of embodiment 63 or embodiment 64, consisting of 1, 2, 3, 4, 5, 6, 7, 8 or 9 reaction vessels.

66. The device of any one of embodiments 63 to 65, wherein one or more reaction vessels are used for single-plex amplification of cDNA.

67. The device of embodiment any one of embodiments 63 to 66, wherein one or more reaction vessels are used for multiplex amplification of cDNA.

68. The device of embodiment 67, wherein the multiplex amplification is 2-plex, 3-plex, 4-plex, 5-plex or 6-plex.

69. A method for inhibiting the development or progression of OPC in a human subject, the method comprising: exposing the subject to a treatment regimen for OPC at least in part on the basis that the subject is determined by the indicator-determining method of any of embodiments 1 to 38 as having a likelihood of a presence of an OPC condition selected from HPV-positive OPC, HPV-negative OPC and HPV-agnostic OPC.

70. The method of embodiment 69, further comprising: taking a sample from the subject and determining an indicator indicative of a likelihood of a presence of the OPC condition using the indicator-determining method.

71. The method of embodiment 69 or embodiment 70, further comprising: sending a sample obtained from the subject to a laboratory at which the indicator is determined according to the indicator-determining method.

72. The method of embodiment 71, further comprising: receiving the indicator from the laboratory.

73. A kit for determining an indicator used in assessing a likelihood that HPV-positive OPC or OPC-negative HPV infection is present or absent in a human subject, the kit comprising: for each of at least one miRNA biomarker (e.g., 1, 2 or 3 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-194-5p, Hsa-miR-501-3p and Hsa-miR-548K.

74. A kit for determining an indicator used in assessing a likelihood that HPV-positive OPC or OPC-negative HPV infection is present or absent in a human subject, the kit comprising: for each of at least one miRNA biomarker (e.g., 1, 2, 3, 4, 5, 6, 7, 8 or 9 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K.

75. A kit for determining an indicator used in assessing a likelihood that HPV-negative OPC or an OPC-negative, HPV-negative condition is present or absent in a human subject, the kit comprising: for each of at least one miRNA biomarker (e.g., 1, 2, 3, 4, 5, 6 or 7 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K.

76. A kit for determining an indicator used in assessing a likelihood that HPV-negative OPC or an OPC-negative, HPV-negative condition is present or absent in a human subject, the kit comprising: for each of at least one miRNA biomarker (e.g., 1, 2, 3, 4, 5 or 6 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-194-5p, Hsa-miR-449a, Hsa-miR-07-5p, Hsa-miR-501-3p, Hsa-miR-1290 and Hsa-miR-548K.

77. A kit for determining an indicator used in assessing a likelihood that HPV-agnostic OPC or an OPC-negative condition is present or absent in a human subject, the kit comprising: for each of at least one miRNA biomarker (e.g., 1, 2, 3 or 4 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-3614-5p, Hsa-miR-501-3p, Hsa-miR-1290 and Hsa-miR-1246.

78. A kit for determining an indicator used in assessing a likelihood that HPV-agnostic OPC or an OPC-negative condition (e.g., HPV-positive, OPC-negative condition, HPV-negative, OPC-negative condition) is present or absent in a human subject, the kit comprising: for each of at least one miRNA biomarker (e.g., 1, 2, 3, 4, 5 or 6 miRNA biomarkers) at least one oligonucleotide primer and/or at least one oligonucleotide probe that hybridizes to the miRNA biomarker or to a cDNA corresponding to the miRNA biomarker, wherein the at least one biomarker is selected from Hsa-miR-449a, Hsa-miR-3614-5p, Hsa-miR-07-5p, Hsa-miR-501-3p, Hsa-miR-1290 and Hsa-miR-548K.

79. A kit for determining an indicator used in assessing a likelihood of a human subject with OPC having a survival prognosis selected from a decreased or poor survival prognosis and an increased or good survival prognosis, the kit comprising: at least one oligonucleotide primer and/or at least one oligonucleotide probe that hybridizes to Hsa-miR-07-5p or to a cDNA corresponding to Hsa-miR-07-5p.

80. The kit of any one of embodiments 73 to 79, wherein the kit comprises for each of at least 2, 3, 4, 5, 6, 7, 8, 9, 10, 11, 12, 13 or 14 of the miRNA biomarkers at least one oligonucleotide primer and/or probe that hybridizes to the miRNA biomarker or a cDNA corresponding to the miRNA biomarker.

81. The kit of any one of embodiments 73 to 79, wherein the kit comprises for each of up to 2, 3, 4, 5, 6, 7, 8, 9, 10, 11, 12, 13 or 14 of the miRNA biomarkers at least one oligonucleotide primer and/or probe that hybridizes to the miRNA biomarker or a cDNA corresponding to the miRNA biomarker.

82. The kit of any one of embodiments 73 to 81, further comprising: a DNA polymerase.

83. The kit of embodiment 82, wherein the DNA polymerase is a thermostable DNA polymerase.

84. The kit of any one of embodiments 73 to 83, further comprising: for each miRNA biomarker a pair of forward and reverse oligonucleotide primers that permit nucleic acid amplification of at least a portion of a cDNA corresponding to the miRNA biomarker, to produce an amplicon.

85. The kit of any one of embodiments 73 to 84, further comprising: for each miRNA biomarker an oligonucleotide probe that comprises a heterologous label and hybridizes to a cDNA corresponding to the miRNA biomarker or to an amplicon of the cDNA.

86. The kit of any one of embodiments 73 to 85, wherein the components of the kit when used to determine the indicator are combined to form a mixture.

87. The kit of any one of embodiments 73 to 86, further comprising: one or more reagents for preparing mRNA from a cell or cell population from a saliva sample.

88. The kit of any one of embodiments 73 to 87, further comprising: one or more reagents for preparing cDNA from the mRNA.

89. The kit of any one of embodiments 73 to 88, further comprising: one or more reagents for amplifying cDNA.

90. The kit of any one of embodiments 73 to 89, further comprising one or more of deoxynucleotides, buffer(s), positive and negative controls, and reaction vessel(s).

91. The kit of any one of embodiments 73 to 90, further comprising instructions for performing the indicator-determining method of any one of embodiments 1 to 38.

In order that the disclosure may be readily understood and put into practical effect, particular preferred embodiments will now be described by way of the following non-limiting examples.

### EXAMPLES

### Example 1

Identification of MIRNA Biomarkers for Discriminating HPV-Positive OPC from Cancer-Free Individuals

Global profiling of salivary miRNA was carried out by small RNA sequencing in order to discover candidate miRNAs that are capable of discriminating HPV-positive OPC from cancer-free individuals. Salivary small RNA isolated from HPV-positive OPC patients (N=6), HPV-negative controls (N=6), and HPV-positive controls (N=4) were considered in the discovery phase.

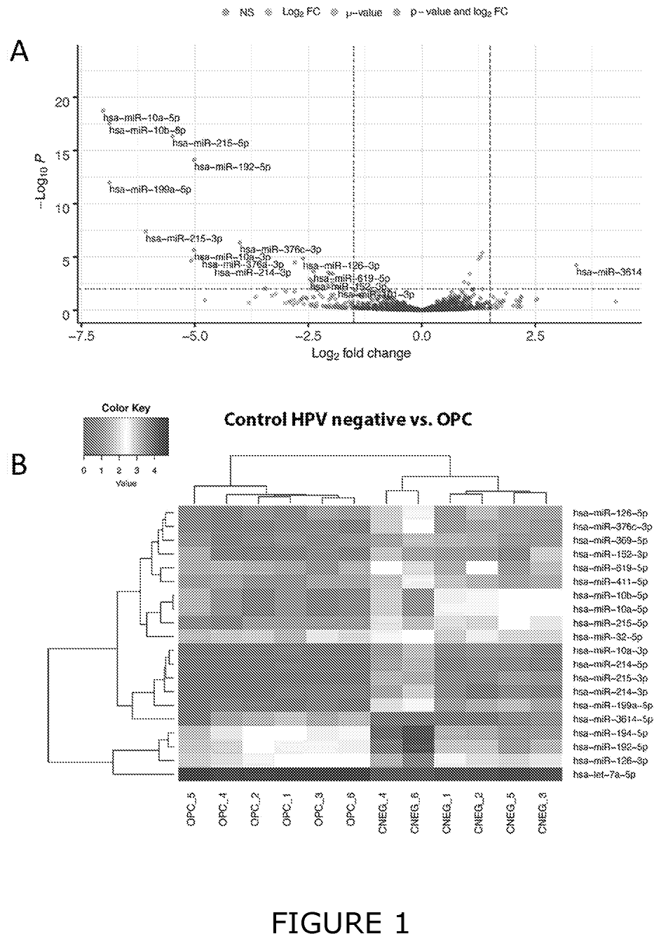

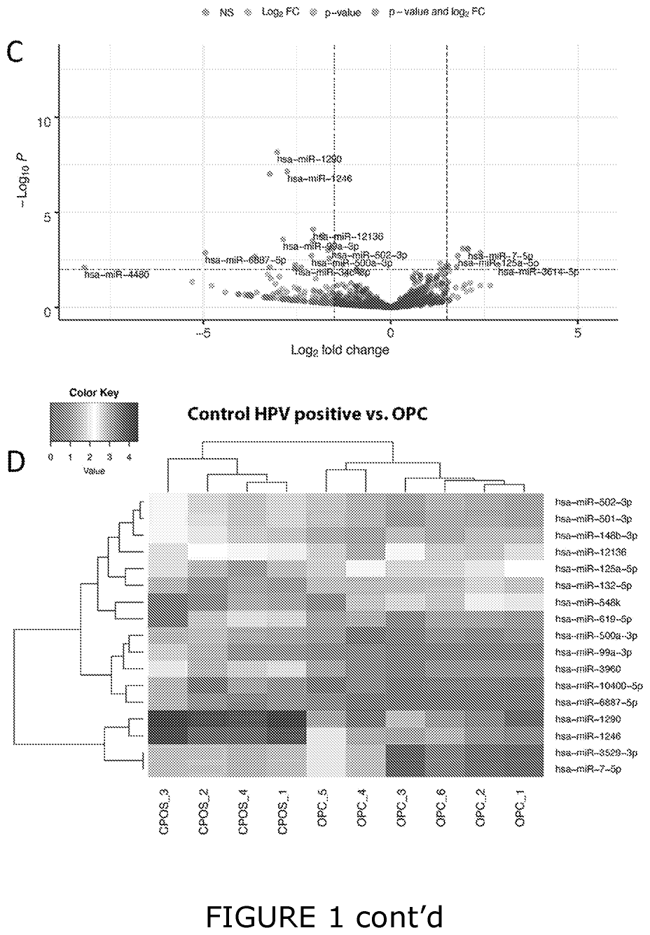

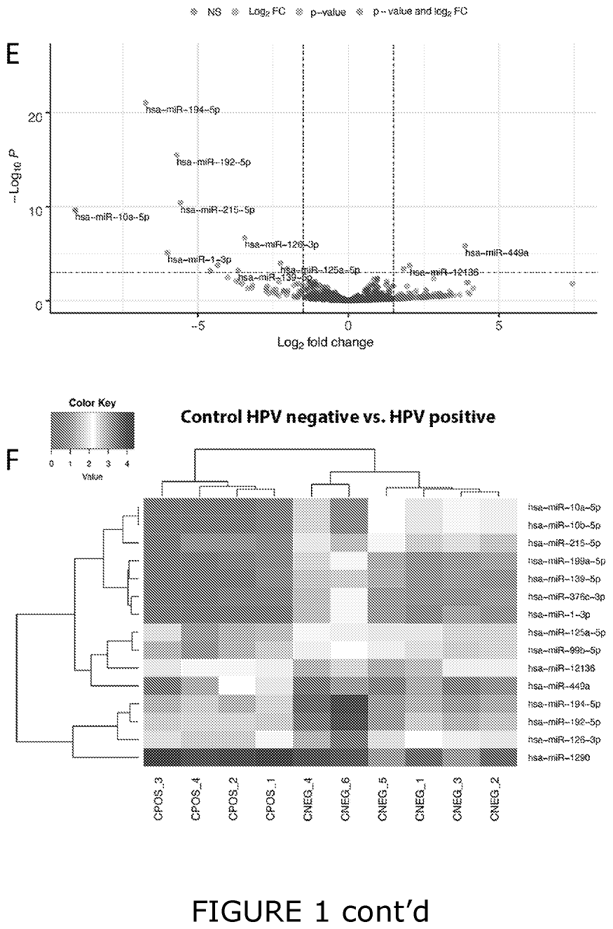

Differently expressed miRNAs discovered by small RNA sequencing are summarized in FIG. 1. DESeq2 algorithm identified 17 differentially expressed miRNAs between HPV-positive OPC and HPV-positive controls (FIG. 1 C-D, TABLE 1).

TABLE 1
Differentially expressed miRNAs comparing Control (HPV-positive) vs OPC. DESeq
package was used to identify differentially expressed miRNAs and estimated normalized
expression counts. Only miRNAs showing an adjusted FDR < 0.05 are shown.

| miRNA | CPOS_1 | CPOS_2 | CPOS_3 | CPOS_4 | OPC_1 | OPC_2 | OPC_3 | OPC_4 | OPC_5 | OPC_6 |
|---|---|---|---|---|---|---|---|---|---|---|
| hsa-miR-1290 | 20571.65 | 10839.35 | 27571.79 | 8260.03 | 4167.56 | 2134.13 | 648.05 | 2943.73 | 1027.55 | 1420.88 |
| hsa-miR-1246 | 8215.91 | 4184.11 | 13808.17 | 4230.35 | 2009.51 | 1606.63 | 1023.69 | 997.59 | 285.55 | 826.81 |
| hsa-miR-3960 | 61.56 | 23.31 | 89.08 | 43.39 | 5.61 | 6.22 | 1.43 | 2.80 | 10.82 | 6.64 |
| hsa-miR-12136 | 261.63 | 142.92 | 334.07 | 206.09 | 67.61 | 32.23 | 126.65 | 15.41 | 45.43 | 38.17 |
| hsa-miR-501-3p | 48.37 | 59.80 | 111.36 | 35.25 | 11.91 | 13.57 | 9.40 | 21.02 | 30.29 | 14.93 |
| hsa-miR-99a-3p | 6.60 | 18.24 | 44.54 | 5.42 | 1.40 | 1.70 | 1.12 | 2.80 | 4.33 | 1.66 |
| hsa-miR-619-5p | 48.37 | 33.45 | 0.00 | 67.79 | 7.71 | 12.63 | 3.50 | 16.81 | 10.82 | 12.03 |
| hsa-miR-132-5p | 8.79 | 5.07 | 22.27 | 10.85 | 34.33 | 51.45 | 27.56 | 23.82 | 25.96 | 29.04 |
| hsa-miR-3529-3p | 670.55 | 617.28 | 623.59 | 799.97 | 4296.84 | 3871.93 | 5596.37 | 638.91 | 298.53 | 2353.89 |
| hsa-miR-502-3p | 54.96 | 54.73 | 111.36 | 32.54 | 12.61 | 16.58 | 12.59 | 18.21 | 34.61 | 21.57 |
| hsa-miR-7-5p | 670.55 | 617.28 | 623.59 | 799.97 | 4296.84 | 3871.93 | 5596.37 | 638.91 | 298.53 | 2353.89 |
| hsa-miR-148b-3p | 35.18 | 88.18 | 133.63 | 37.96 | 24.17 | 26.01 | 28.67 | 9.81 | 15.14 | 14.52 |
| hsa-miR-548k | 13.19 | 4.05 | 0.00 | 16.27 | 85.13 | 99.88 | 56.39 | 30.82 | 4.33 | 36.09 |
| hsa-miR-6887-5p | 5.50 | 5.07 | 22.27 | 2.71 | 0.00 | 0.00 | 0.00 | 0.00 | 4.33 | 0.00 |
| hsa-miR-125a-5p | 28.58 | 22.30 | 66.81 | 8.14 | 167.81 | 79.72 | 50.18 | 116.29 | 38.94 | 56.84 |
| hsa-miR-500a-3p | 13.19 | 27.37 | 22.27 | 13.56 | 5.96 | 3.96 | 2.39 | 0.00 | 8.65 | 5.81 |
| hsa-miR-10400-5p | 6.60 | 0.00 | 22.27 | 10.85 | 0.35 | 0.38 | 0.32 | 2.80 | 0.00 | 0.41 |

| | | | | | | | OPC |
|---|---|---|---|---|---|---|---|
| miRNA | baseMean | log2FoldChange | lfcSE | stat | pvalue | padj | expression? |
| hsa-miR-1290 | 7958.47 | −3.030 | 0.523 | −5.796590138 | 6.77E−09 | 2.36E−06 | Down |
| | | | | | | | regulated |
| hsa-miR-1246 | 3718.83 | −2.754 | 0.511 | −5.38969967 | 7.06E−08 | 1.08E−05 | Down |
| | | | | | | | regulated |
| hsa-miR-3960 | 25.09 | −3.223 | 0.604 | −5.340080476 | 9.29E−08 | 1.08E−05 | Down |
| | | | | | | | regulated |
| hsa-miR-12136 | 127.02 | −2.068 | 0.524 | −3.944262071 | 8.00E−05 | 0.006984021 | Down |
| | | | | | | | regulated |
| hsa-miR-501-3p | 35.59 | −1.816 | 0.481 | −3.775196193 | 0.000159882 | 0.011159748 | Down |
| | | | | | | | regulated |
| hsa-miR-99a-3p | 8.78 | −2.868 | 0.785 | −3.65097931 | 0.000261242 | 0.015195593 | Down |
| | | | | | | | regulated |
| hsa-miR-619-5p | 21.31 | −2.077 | 0.579 | −3.588574228 | 0.000332491 | 0.016577068 | Down |
| | | | | | | | regulated |
| hsa-miR-132-5p | 23.91 | 1.937 | 0.579 | 3.34831372 | 0.000813049 | 0.026698499 | Up regulated |
| hsa-miR-3529-3p | 1976.79 | 2.067 | 0.619 | 3.338770694 | 0.0008415 | 0.026698499 | Up regulated |
| hsa-miR-502-3p | 36.98 | −1.577 | 0.469 | −3.365746903 | 0.000763367 | 0.026698499 | Down |
| | | | | | | | regulated |
| hsa-miR-7-5p | 1976.79 | 2.067 | 0.619 | 3.338770694 | 0.0008415 | 0.026698499 | Up regulated |
| hsa-miR-148b-3p | 41.33 | −1.672 | 0.507 | −3.295583021 | 0.000982177 | 0.028564975 | Down |
| | | | | | | | regulated |
| hsa-miR-548k | 34.62 | 2.404 | 0.751 | 3.202461151 | 0.001362587 | 0.033973635 | Up regulated |
| hsa-miR-6887-5p | 3.99 | −4.950 | 1.546 | −3.202407794 | 0.001362839 | 0.033973635 | Down |
| | | | | | | | regulated |
| hsa-miR-125a-5p | 63.56 | 1.795 | 0.579 | 3.097943892 | 0.001948683 | 0.042573495 | Up regulated |
| hsa-miR-500a-3p | 10.32 | −2.114 | 0.682 | −3.097471217 | 0.001951793 | 0.042573495 | Down |
| | | | | | | | regulated |
| hsa-miR-10400-5p | 4.40 | −3.619 | 1.178 | −3.073050535 | 0.002118826 | 0.043498252 | Down |
| | | | | | | | regulated |

The strongest association was observed for hsa-miR-1290 (P=2.36*10⁻⁰⁶, logFC=−3.030). Similarly, expression differences were identified for 24 miRNAs between HPV-positive OPC and HPV-negative controls (FIG. 1A-B, TABLE 2) where hsa-miR-10a-5p (P=1.85*10⁻¹⁹, logFC=−7.018) was observed to be the leading candidate.

TABLE 2
Differentially expressed miRNAs comparing Control (HPV-negative) vs OPC. DESeq package was used to identify differentially
expressed miRNAs and estimated normalized expression counts. Only miRNAs showing an adjusted FDR < 0.05 are shown.

| miRNA | CNEG_1 | CNEG_2 | CNEG_3 | CNEG_4 | CNEG_5 | CNEG_6 | OPC_1 | OPC_2 | OPC_3 | OPC_4 |
|---|---|---|---|---|---|---|---|---|---|---|
| hsa-miR-10a-5p | 157.77 | 167.20 | 218.76 | 765.75 | 274.41 | 3103.02 | 6.51 | 0.98 | 2.30 | 6.91 |
| hsa-miR-10b-5p | 150.44 | 164.09 | 215.95 | 765.75 | 270.61 | 3096.41 | 6.51 | 0.98 | 2.30 | 6.91 |
| hsa-miR-194-5p | 1564.90 | 1686.57 | 2293.20 | 7993.04 | 2710.93 | 22781.90 | 244.60 | 219.57 | 142.41 | 119.72 |
| hsa-miR-215-5p | 80.72 | 65.43 | 119.66 | 496.84 | 251.12 | 1143.39 | 11.06 | 10.50 | 8.33 | 2.30 |
| hsa-miR-192-5p | 917.29 | 938.83 | 1319.08 | 4909.54 | 2456.89 | 16450.29 | 160.68 | 252.39 | 157.62 | 66.77 |
| hsa-miR-199a-5p | 9.17 | 20.77 | 15.89 | 99.88 | 34.55 | 363.51 | 0.00 | 0.66 | 1.44 | 2.30 |
| hsa-miR-215-3p | 3.67 | 3.12 | 7.48 | 25.61 | 9.75 | 19.83 | 0.00 | 0.00 | 0.00 | 0.00 |
| hsa-miR-376c-3p | 9.17 | 15.58 | 8.41 | 51.22 | 14.05 | 218.10 | 3.90 | 2.30 | 4.88 | 2.30 |
| hsa-miR-10a-3p | 3.67 | 6.23 | 2.80 | 10.24 | 5.95 | 36.35 | 0.65 | 0.33 | 0.00 | 0.00 |
| hsa-let-7e-5p | 11612.93 | 10908.68 | 9626.20 | 6515.32 | 14727.32 | 7032.19 | 26540.56 | 19192.13 | 24975.82 | 19155.99 |
| hsa-let-7f-5p | 17778.97 | 17976.89 | 16137.47 | 12095.85 | 27611.62 | 12574.00 | 39827.42 | 34502.31 | 46314.26 | 30343.37 |
| hsa-miR-376a-3p | 3.67 | 3.12 | 2.80 | 10.24 | 4.43 | 59.48 | 0.00 | 0.00 | 0.86 | 0.00 |
| hsa-let-7a-5p | 21248.17 | 19466.14 | 17740.74 | 13711.87 | 30769.85 | 13885.92 | 42683.86 | 36218.14 | 48818.42 | 34593.60 |
| hsa-miR-126-3p | 354.07 | 523.42 | 450.60 | 1516.14 | 642.35 | 4421.55 | 276.47 | 356.42 | 254.66 | 103.61 |
| hsa-miR-214-5p | 1.83 | 4.15 | 0.93 | 12.81 | 3.16 | 29.74 | 0.65 | 0.00 | 0.00 | 0.00 |
| hsa-miR-126-5p | 9.17 | 36.35 | 16.83 | 61.47 | 14.30 | 158.62 | 3.90 | 5.91 | 13.21 | 2.30 |
| hsa-miR-3614-5p | 0.00 | 0.00 | 6.54 | 0.00 | 3.29 | 0.00 | 45.54 | 22.97 | 17.51 | 16.12 |
| hsa-miR-214-3p | 3.67 | 0.00 | 2.80 | 15.37 | 3.80 | 59.48 | 0.65 | 0.00 | 0.86 | 0.00 |
| hsa-miR-619-5p | 20.18 | 203.55 | 37.39 | 235.62 | 10.76 | 82.61 | 14.31 | 21.99 | 6.32 | 27.63 |
| hsa-miR-411-5p | 42.20 | 27.00 | 19.63 | 40.98 | 16.07 | 158.62 | 5.85 | 7.55 | 15.79 | 13.81 |
| hsa-miR-32-5p | 51.37 | 136.05 | 624.48 | 361.11 | 76.45 | 234.63 | 26.02 | 53.17 | 127.19 | 46.05 |
| hsa-miR-369-5p | 29.35 | 18.69 | 11.22 | 12.81 | 8.61 | 39.66 | 5.20 | 3.28 | 4.31 | 2.30 |
| hsa-let-7c-5p | 27775.62 | 18305.07 | 15768.20 | 13591.50 | 25314.84 | 12289.80 | 37344.39 | 27435.19 | 36845.73 | 32641.16 |
| hsa-miR-152-3p | 11.01 | 11.42 | 57.03 | 43.54 | 3.54 | 13.22 | 1.95 | 0.98 | 4.88 | 2.30 |

| | | | | | | | | | OPC |
|---|---|---|---|---|---|---|---|---|---|
| miRNA | OPC_5 | OPC_6 | baseMean | log2FoldChange | lfcSE | stat | pvalue | padj | expression? |
| hsa-miR-10a-5p | 23.29 | 3.35 | 394.19 | −7.018 | 0.778 | −9.022 | 1.85E−19 | 1.24E−16 | Down |
| | | | | | | | | | regulated |
| hsa-miR-10b-5p | 27.17 | 3.35 | 392.54 | −6.885 | 0.789 | −8.724 | 2.70E−18 | 9.07E−16 | Down |
| | | | | | | | | | regulated |
| hsa-miR-194-5p | 34.93 | 180.09 | 3330.99 | −5.366 | 0.627 | −8.564 | 1.09E−17 | 2.45E−15 | Down |
| | | | | | | | | | regulated |
| hsa-miR-215-5p | 3.88 | 8.38 | 183.47 | −5.494 | 0.654 | −8.401 | 4.44E−17 | 7.47E−15 | Down |
| | | | | | | | | | regulated |
| hsa-miR-192-5p | 46.57 | 145.75 | 2318.48 | −5.014 | 0.644 | −7.781 | 7.22E−15 | 9.71E−13 | Down |
| | | | | | | | | | regulated |
| hsa-miR-199a-5p | 0.00 | 0.00 | 45.68 | −6.880 | 0.965 | −7.132 | 9.86E−13 | 1.11E−10 | Down |
| | | | | | | | | | regulated |
| hsa-miR-215-3p | 0.00 | 0.00 | 5.79 | −6.081 | 1.108 | −5.490 | 4.03E−08 | 3.87E−06 | Down |
| | | | | | | | | | regulated |
| hsa-miR-376c-3p | 3.88 | 1.68 | 27.96 | −4.006 | 0.792 | −5.057 | 4.25E−07 | 3.58E−05 | Down |
| | | | | | | | | | regulated |
| hsa-miR-108-3p | 0.00 | 0.00 | 5.52 | −5.015 | 1.060 | −4.733 | 2.21E−06 | 0.000165071 | Down |
| | | | | | | | | | regulated |
| hsa-let-7e-5p | 27874.23 | 34512.24 | 17722.80 | 1.333 | 0.290 | 4.600 | 4.23E−06 | 0.00028494 | Up regulated |
| hsa-let-7f-5p | 44423.35 | 58105.25 | 29807.90 | 1.283 | 0.290 | 4.426 | 9.59E−06 | 0.000586437 | Up regulated |
| hsa-miR-3768-3p | 0.00 | 0.84 | 7.12 | −4.841 | 1.103 | −4.388 | 1.14E−05 | 0.000641923 | Down |
| | | | | | | | | | regulated |
| hsa-let-7a-5p | 53392.63 | 62164.24 | 32891.13 | 1.250 | 0.290 | 4.315 | 1.60E−05 | 0.000768632 | Up regulated |
| hsa-miR-126-3p | 50.45 | 239.56 | 765.78 | −2.616 | 0.605 | −4.325 | 1.53E−05 | 0.000768632 | Down |
| | | | | | | | | | regulated |
| hsa-miR-214-5p | 0.00 | 0.00 | 4.44 | −5.076 | 1.197 | −4.242 | 2.21E−05 | 0.000993568 | Down |
| | | | | | | | | | regulated |
| hsa-miR-126-5p | 3.88 | 9.21 | 27.93 | −2.801 | 0.677 | −4.135 | 3.54E−05 | 0.001489855 | Down |
| | | | | | | | | | regulated |
| hsa-miR-3614-5p | 0.00 | 30.99 | 11.91 | 3.405 | 0.850 | 4.007 | 6.14E−05 | 0.002432549 | Up regulated |
| hsa-miR-214-3p | 0.00 | 0.84 | 7.29 | −4.567 | 1.147 | −3.983 | 6.81E−05 | 0.002544557 | Down |
| | | | | | | | | | regulated |
| hsa-miR-619-5p | 19.41 | 24.29 | 58.67 | −2.386 | 0.649 | −3.678 | 0.000234701 | 0.008313358 | Down |
| | | | | | | | | | regulated |
| hsa-miR-411-5p | 11.64 | 15.92 | 31.26 | −2.066 | 0.575 | −3.592 | 0.000327942 | 0.011035256 | Down |
| | | | | | | | | | regulated |
| hsa-miR-32-5p | 31.05 | 77.90 | 153.79 | −2.019 | 0.568 | −3.551 | 0.000383134 | 0.012278524 | Down |
| | | | | | | | | | regulated |
| hsa-miR-369-5p | 7.76 | 7.54 | 12.56 | −1.961 | 0.555 | −3.533 | 0.000410315 | 0.012551915 | Down |
| | | | | | | | | | regulated |
| hsa-let-7c-5p | 51715.98 | 48860.92 | 28990.70 | 1.055 | 0.301 | 3.507 | 0.000452635 | 0.013244485 | Up regulated |
| hsa-miR-152-3p | 19.41 | 2.51 | 14.32 | −2.461 | 0.767 | −3.208 | 0.001338044 | 0.037520994 | Down |
| | | | | | | | | | regulated |

Comparison between HPV-positive controls and HPV-negative controls identified 15 miRNAs discriminating these groups (FIG. 1E-F, TABLE 3). The strongest association was observed with hsa-miR-194-5p (P=3.20*10⁻¹⁹, logFC=−6.729).

TABLE 3
Differentially expressed miRNAs comparing Control (HPV-negative) versus Control (HPV-positive).
DESeq package was used to identify differentially expressed miRNAs and estimated normalized
expression counts. Only miRNAs showing an adjusted FDR < 0.05 are shown.

| miRNA | CNEG_1 | CNEG_2 | CNEG_3 | CNEG_4 | CNEG_5 | CNEG_6 | CPOS_1 | CPOS_2 | CPOS_3 | CPOS_4 |
|---|---|---|---|---|---|---|---|---|---|---|
| hsa-miR-194-5p | 723.73 | 838.02 | 1144.39 | 3978.01 | 1248.67 | 11614.78 | 43.86 | 17.82 | 18.27 | 36.97 |
| hsa-miR-192-5p | 424.23 | 466.48 | 658.27 | 2443.40 | 1131.66 | 8386.76 | 37.46 | 43.28 | 36.55 | 53.14 |
| hsa-miR-215-5p | 37.33 | 32.51 | 59.72 | 247.27 | 115.67 | 582.93 | 2.74 | 4.24 | 0.00 | 4.62 |
| hsa-miR-10a-5p | 72.97 | 83.08 | 109.17 | 381.10 | 126.39 | 1582.00 | 0.91 | 0.00 | 0.00 | 0.00 |
| hsa-miR-10b-5p | 69.57 | 81.53 | 107.77 | 381.10 | 124.65 | 1578.63 | 0.91 | 0.00 | 0.00 | 0.00 |
| hsa-miR-126-3p | 163.75 | 260.07 | 224.86 | 754.56 | 295.87 | 2254.22 | 112.38 | 36.49 | 54.82 | 34.66 |
| hsa-miR-449a | 12.73 | 4.64 | 1.87 | 0.00 | 1.52 | 8.42 | 73.09 | 165.47 | 0.00 | 13.86 |
| hsa-miR-1-3p | 3.39 | 6.19 | 9.80 | 28.04 | 6.88 | 119.62 | 0.00 | 0.00 | 0.00 | 0.00 |
| hsa-miR-125a-5p | 78.91 | 52.12 | 44.79 | 104.52 | 77.42 | 234.18 | 23.76 | 18.67 | 54.82 | 6.93 |
| hsa-miR-12136 | 23.76 | 93.92 | 83.51 | 17.84 | 12.83 | 47.17 | 217.45 | 119.65 | 274.12 | 175.60 |
| hsa-miR-376c-3p | 4.24 | 7.74 | 4.20 | 25.49 | 6.47 | 111.19 | 0.91 | 1.70 | 0.00 | 0.00 |
| hsa-miR-1290 | 3525.31 | 3518.23 | 1270.35 | 7902.49 | 1189.84 | 6124.12 | 17098.37 | 9074.77 | 22623.71 | 7037.85 |
| hsa-miR-99b-5p | 66.18 | 46.44 | 41.52 | 94.32 | 88.32 | 168.48 | 36.55 | 18.67 | 18.27 | 4.62 |
| hsa-miR-139-5p | 4.24 | 5.16 | 8.40 | 29.32 | 15.27 | 38.75 | 2.74 | 0.00 | 0.00 | 0.00 |
| hsa-miR-199a-5p | 4.24 | 10.32 | 7.93 | 49.71 | 15.92 | 185.32 | 3.65 | 0.85 | 0.00 | 0.00 |

| | | | | | | | CPOS |
|---|---|---|---|---|---|---|---|
| miRNA | baseMean | log2FoldChange | lfcSE | stat | pvalue | padj | expression? |
| hsa-miR-194-5p | 1966.45 | −6.729 | 0.702 | −9.587776722 | 9.00E−22 | 3.20E−19 | Down |
| | | | | | | | regulated |
| hsa-miR-192-5p | 1368.12 | −5.704 | 0.699 | −8.160606444 | 3.33E−16 | 5.92E−14 | Down |
| | | | | | | | regulated |
| hsa-miR-215-5p | 108.70 | −5.577 | 0.844 | −6.605088072 | 3.97E−11 | 4.70E−09 | Down |
| | | | | | | | regulated |
| hsa-miR-10a-5p | 235.56 | −9.074 | 1.432 | −6.3382615 | 2.32E−10 | 2.01E−08 | Down |
| | | | | | | | regulated |
| hsa-miR-10b-5p | 234.42 | −9.067 | 1.437 | −6.307821062 | 2.83E−10 | 2.01E−08 | Down |
| | | | | | | | regulated |
| hsa-miR-126-3p | 419.17 | −3.445 | 0.663 | −5.200145317 | 1.99E−07 | 1.18E−05 | Down |
| | | | | | | | regulated |
| hsa-miR-449a | 28.16 | 3.879 | 0.806 | 4.812074856 | 1.49E−06 | 7.58E−05 | Up regulated |
| hsa-miR-1-3p | 17.39 | −6.003 | 1.343 | −4.46828518 | 7.88E−06 | 3.50E−04 | Down |
| | | | | | | | regulated |
| hsa-miR-125a-5p | 69.61 | −2.253 | 0.580 | −3.88491068 | 0.000102368 | 0.00403783 | Down |
| | | | | | | | regulated |
| hsa-miR-12136 | 106.58 | 2.044 | 0.545 | 3.749322375 | 1.77E−04 | 0.005722375 | Up regulated |
| hsa-miR-376c-3p | 16.19 | −4.339 | 1.155 | −3.758177283 | 0.000171156 | 0.005722375 | Down |
| | | | | | | | regulated |
| hsa-miR-1290 | 7936.50 | 1.831 | 0.519 | 3.528264928 | 0.000418293 | 0.011422624 | Up regulated |
| hsa-miR-99b-5p | 58.34 | −2.018 | 0.569 | −3.548467363 | 0.00038748 | 0.011422624 | Down |
| | | | | | | | regulated |
| hsa-miR-139-5p | 10.39 | −3.659 | 1.068 | −3.426327585 | 0.000611802 | 0.015513552 | Down |
| | | | | | | | regulated |
| hsa-miR-199a-5p | 27.79 | −4.593 | 1.355 | −3.390198223 | 0.000698421 | 0.016529298 | Down |
| | | | | | | | regulated |

### Example 2

### Validation of MIRNA Biomarkers by Quantitative PCR

Following initial screening for the expression levels in saliva and based on the availability of miScript™ primer PCR Assays (QIAGEN, MD, USA), 14 miRNAs were selected for further validation by qPCR. The selected miRNAs were tested in a separate cohort of HPV-positive OPC patients (N=46), HPV-negative controls (N=46), and HPV-positive controls (N=16). In order to compare the expression patterns of these miRNAs between HPV-positive OPC and HPV-negative OPC, an additional cohort of HPV-negative OPC samples (N=14) was also tested. Demographic and clinical information of the study participants is listed in TABLE 4.

TABLE 4
Demographic and clinical characteristics of participants considered for qPCR validation
using miScript ™ primer PCR Assays.

| | | | | HPV-positive | HPV-negative |
|---|---|---|---|---|---|
| | | HPV-positive | HPV-negative | controls | controls |
| | | OPC (N = 46) | OPC (N = 14) | (N = 16) | (N = 46) |
| Age | <=55 | 8 (17.4%) | 3 (21.4%) | 8 (50.0%) | 17 (37.0%) |
| | 56-65 | 21 (45.7%) | 7 (50.0%) | 4 (25.0%) | 14 (30.4%) |
| | >=66 | 17 (37.0%) | 4 (28.6%) | 4 (25.0%) | 15 (32.6%) |
| | Mean (SD) | 61.87 (8.24) | 59.64 (7.92) | 56.56 (12.73) | 58.48 (12.72) |
| Gender | Male | 44 (95.7%) | 10 (71.4%) | 13 (81.2%) | 39 (84.8%) |
| | Female | 2 (4.3%) | 4 (28.6%) | 3 (18.8%) | 7 (15.2%) |
| OPC site | Tonsil | 27 (58.7%) | 4 (28.6%) | — | — |
| | BOT | 13 (28.3%) | 4 (28.6%) | | |
| | Tonsil & BOT | 5 (10.9%) | 0 | | |
| | Other | 1 (2.2%) | 6 (42.9%) | | |
| AJCC stage | Stage 01 | 4 (8.7%) | 0 | — | — |
| (8^{th} Edition) | Stage 02 | 28 (60.9%) | 1 (7.1%) | | |
| | Stage 03 | 12 (26.1%) | 5 (35.7%) | | |
| | Stage 04 | 0 | 6 (42.9%) | | |
| | Not Available | 2 (4.3%) | 2 (14.3%) | | |

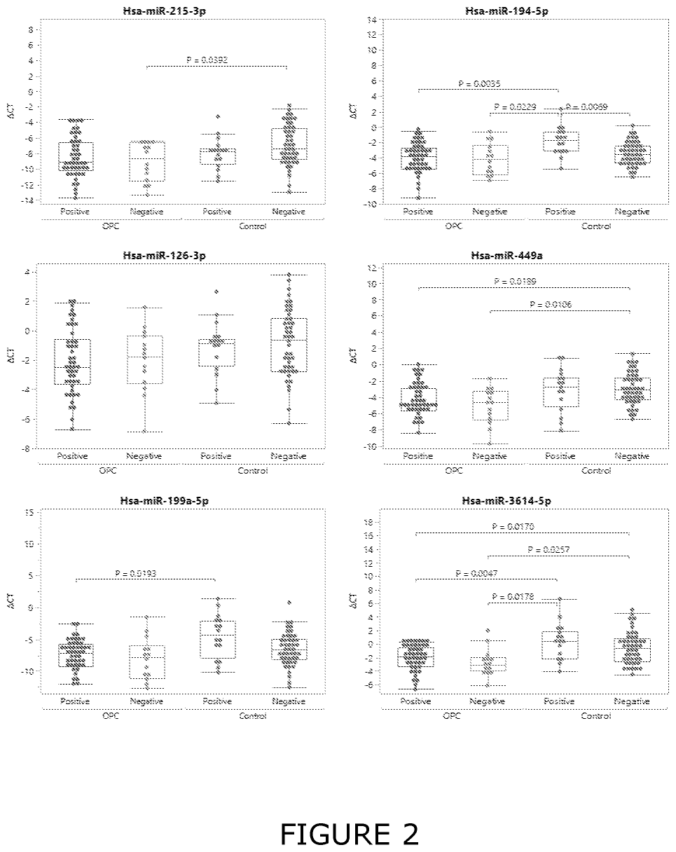

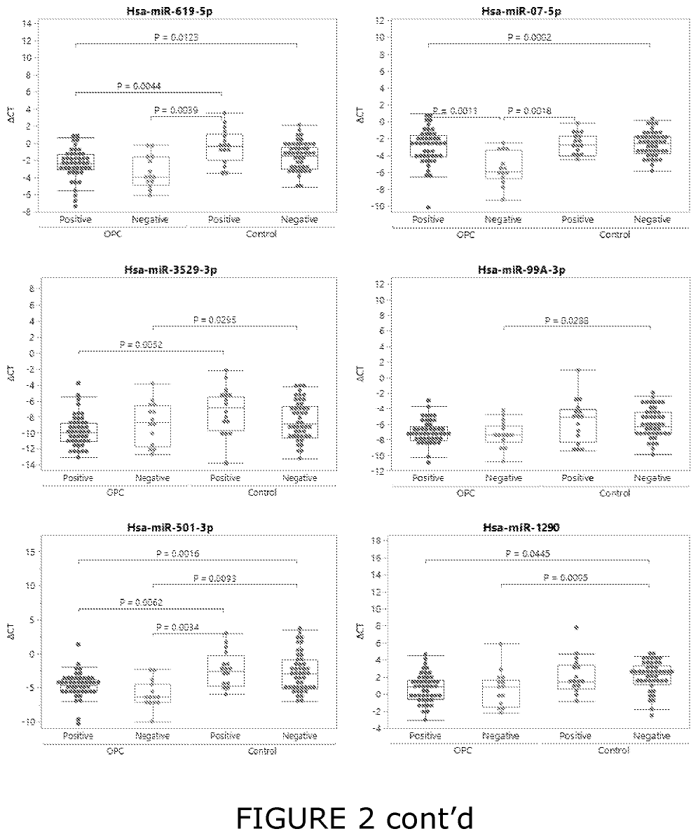

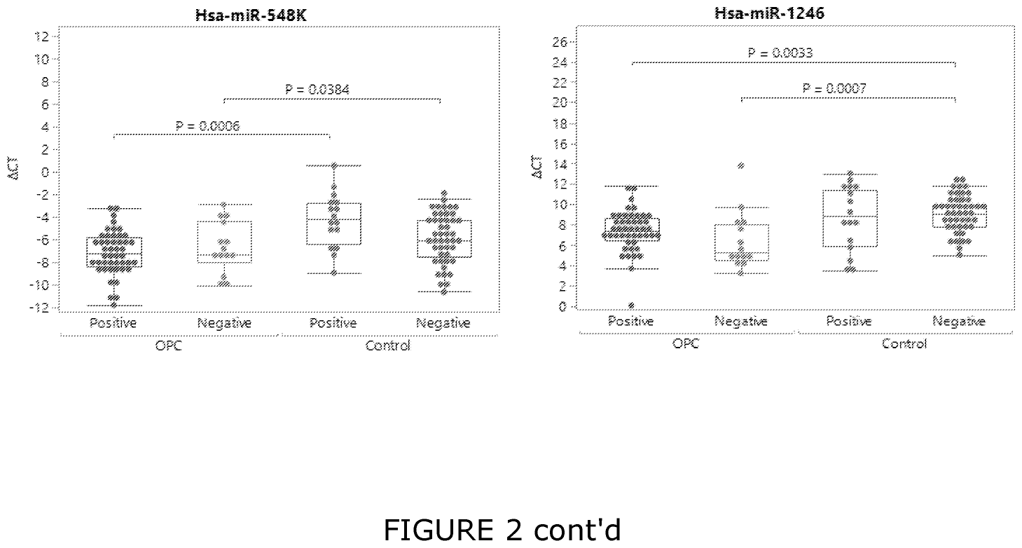

Several miRNAs were validated to be markedly dysregulated in the saliva of OPC patients compared to controls (FIG. 2, TABLE 5). Indicating that the majority of these miRNA expression changes are cancer-driven, a limited number of miRNAs were identified as being differentially expressed between subjects with HPV-positive OPC and OPC-negative, HPV-positive subjects, between subjects with HPV-negative OPC and OPC-negative, HPV-negative subjects, or between subjects with HPV-agnostic OPC and OPC-negative subjects. Significant salivary expression changes were limited to Hsa-miR-07-5p (P=0.0011) between HPV-positive OPC and HPV-negative OPC, and Hsa-miR-194-5p (P=0.0069) between HPV-positive controls and HPV-negative controls.

TABLE 5
One way Kruskal-Wallis analysis with Steel-Dwass pairwise comparison of
salivary miRNA expression - miScript ™ primer PCR Assays.

| | | | | Steel Dwass multiple comparison procedure P VALUES - valid | | | | | |
|---|---|---|---|---|---|---|---|---|---|
| | | | | only if Kruskal Wallis is significant. | | | | | |
| | | | | HPV | HPV | HPV | HPV | HPV | HPV |
| | | | | Positive | Positive | Negative | Positive | Positive | Negative |
| | | | | OPC vs | OPC vs | OPC vs | Control | OPC vs | OPC vs |
| | Kruskal Wallis Test | | | HPV | HPV | HPV | vs HPV | HPV | HPV |
| | Chi- | | Prob > | Negative | Positive | Positive | Negative | Negative | Negative |
| miRNA | square | DF | ChiSq | OPC | Control | Control | Control | Control | Control |
| Hsa-miR-215-3p | 12.9156 | 3 | 0.0250* | 0.9137 | 0.8251 | 0.8861 | 0.5764 | 0.0392* | 0.1202 |
| Hsa-miR-194-5p | 11.29 | 3 | 0.0020* | 0.9510 | 0.0035* | 0.0229* | 0.0069* | 0.7913 | 0.6835 |
| Hsa-miR-126-3p | 6.2508 | 3 | 0.0492* | 0.9971 | 0.3511 | 0.6885 | 0.9407 | 0.0583 | 0.4095 |
| Hsa-miR-449a | 14.3276 | 3 | 0.0024* | 0.8048 | 0.3423 | 0.1403 | 1.0000 | 0.0106* | 0.0189* |
| Hsa-miR-199a-5p | 18.228 | 3 | 0.0084* | 0.9008 | 0.0193* | 0.0576 | 0.1694 | 0.3286 | 0.3992 |
| Hsa-miR-3614-5p | 22.1134 | 3 | 0.0002* | 0.4515 | 0.0047* | 0.0178* | 0.5352 | 0.0257* | 0.0170* |
| Hsa-miR-619-5p | 18.2069 | 3 | 0.0001* | 0.3401 | 0.0044* | 0.0039* | 0.1199 | 0.1541 | 0.0123* |
| Hsa-miR-07-5p | 10.0129 | 3 | 0.0003* | 0.0011* | 1.0000 | 0.0018* | 1.0000 | 0.9932 | 0.0002* |
| Hsa-miR-3529-3p | 16.3767 | 3 | 0.0025* | 0.5504 | 0.0052* | 0.4522 | 0.3251 | 0.0295* | 0.9963 |
| Hsa-miR-99A-3p | 20.5783 | 3 | 0.0101* | 0.9737 | 0.2322 | 0.3105 | 0.9858 | 0.0288* | 0.0833 |
| Hsa-miR-501-3p | 28.1669 | 3 | <.0001* | 0.0984 | 0.0062* | 0.0034* | 0.8251 | 0.0093* | 0.0016* |
| Hsa-miR-1290 | 23.0891 | 3 | 0.0003* | 0.9988 | 0.0890 | 0.3105 | 0.9876 | 0.0005* | 0.0445* |
| Hsa-miR-548K | 23.5909 | 3 | 0.0004* | 0.9198 | 0.0006* | 0.0641 | 0.0856 | 0.6507 | 0.0384* |
| Hsa-miR-1246 | 20.5791 | 3 | 0.0002* | 0.2221 | 0.3782 | 0.2895 | 0.9998 | 0.0007* | 0.0033* |

### Example 3

### Salivary MIRNA Expression—HPV-Positive OPC Vs HPV-Positive Controls

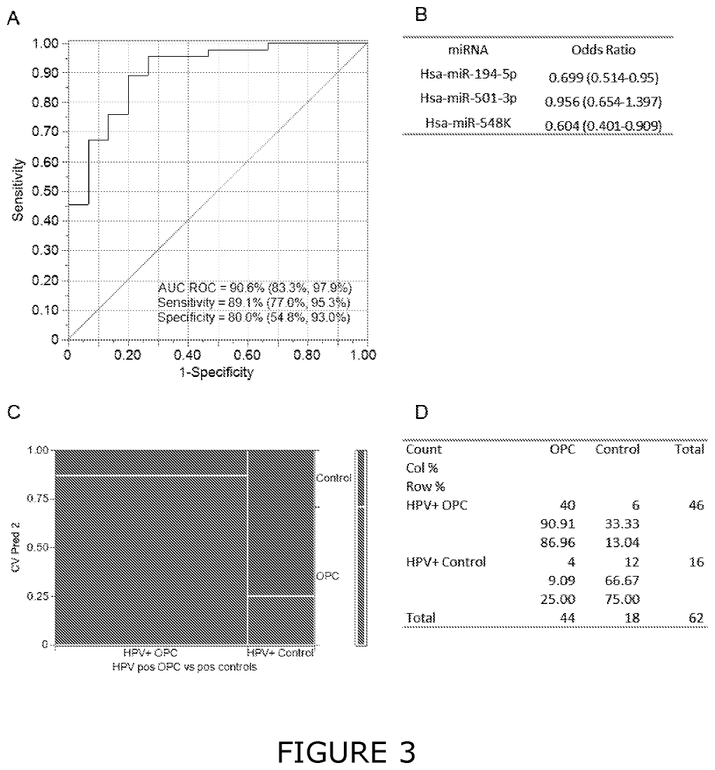

Regression modeling identified a salivary diagnostic biomarker panel consisting of three miRNAs (i.e., Hsa-miR-194-5p, Hsa-miR-501-3p and Hsa-miR-501-3p) with the potential to differentiate HPV-positive OPC patients from HPV-positive controls. The assay had a sensitivity of 89.1% (77.0%, 95.3%) and a specificity of 80.0% (54.8%, 93.0%) (FIG. 3). ROC analysis revealed that the panel has an AUC value of 90.6% (83.3%, 97.9%) (FIG. 3). Leave One Out cross-validation revealed a sensitivity of 87.0% (74.3%, 93.9%) and specificity of 75.0% (50.5%, 89.8%) for the panel to distinguish HPV-positive OPC patients from HPV-positive controls.

### Example 4

### Salivary MIRNA Expression—HPV-Negative OPC Vs HPV-Negative Controls

Although salivary miRNA changes in HPV-negative OPC patients were not investigated at the discovery phase, several miRNAs considered in the validation phase showed differential expression in the saliva of HPV-negative OPC patients compared to HPV-negative controls (TABLE 5). Eight of these miRNA were observed to be significantly dysregulated between these groups including Hsa-miR-07-5p (P=0.0002), displaying the most significant differential expression.

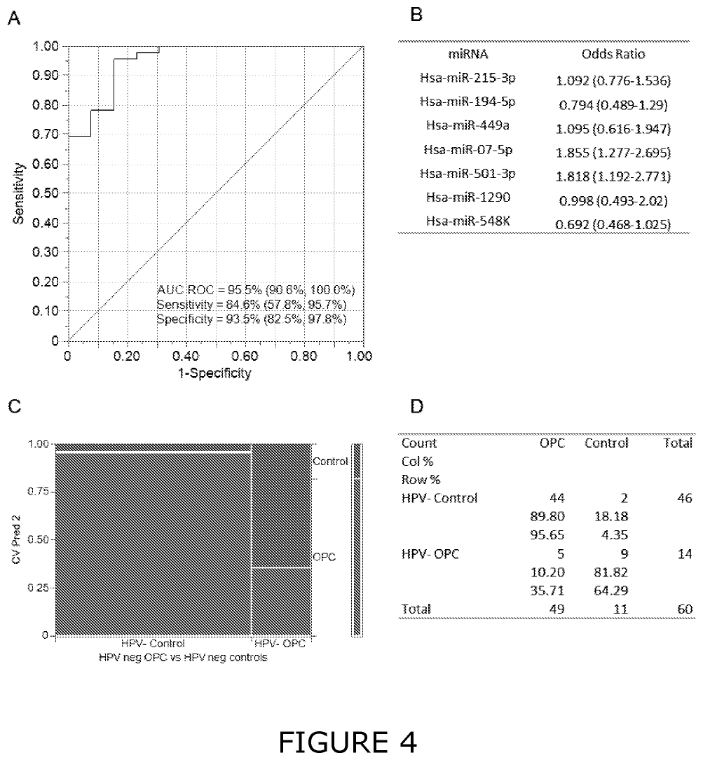

Regression analysis revealed that a salivary biomarker panel consisting of seven miRNAs (i.e., Hsa-miR-215-3p, Hsa-miR-194-5p, Hsa-miR-449a, Hsa-miR-07-5p, Hsa-miR-501-3p, Hsa-miR-1290 and Hsa-miR-548K) could distinguish HPV-negative OPC patients from controls with a sensitivity of 84.6% (57.8%, 95.7%) and a specificity of 93.5% (82.5%, 97.8%) (FIG. 4). The AUC value of the panel was 95.5% (90.6%, 100.0%) (FIG. 4). Cross validation revealed a sensitivity of 84.6% (57.8%, 95.7%) and specificity of 93.5% (82.5%, 97.8%) for the panel to distinguish HPV-negative OPC patients from HPV-negative controls.

### Example 5

### Salivary MIRNA Expression—OPC (HPV-Positive and Negative) Vs Controls (HPV-Positive and Negative)

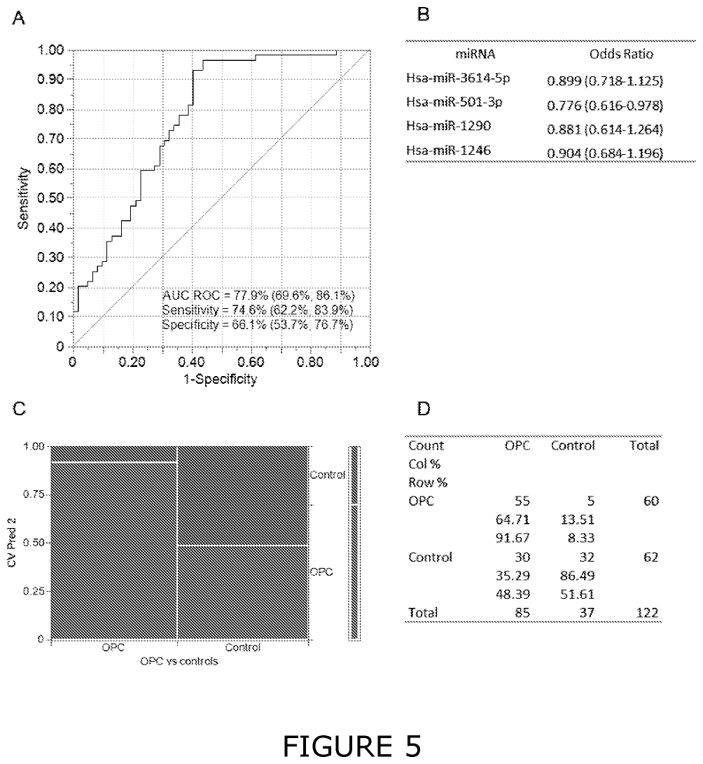

Considering that several miRNAs are associated with both HPV-positive and HPV-negative OPC, overall results were analyzed to identify salivary miRNA candidates capable of predicting OPC irrespective of the HPV status. The analysis identified a panel consisting of four miRNA (i.e., Hsa-miR-3614-5p, Hsa-miR-501-3p, Hsa-miR-1290 and Hsa-miR-1246) (FIG. 5) that is capable of differentiating OPC patients from controls with a sensitivity of 74.6% (62.2%, 83.9%) and a specificity of 66.1% (53.7%, 76.7%) The AUC of the panel was 77.9% (69.6%, 86.1%). Cross-validation revealed a sensitivity of 91.7% (81.9%, 96.4%) and specificity of 51.6% (39.4%, 63.6%) for the assay to distinguish OPC patients from controls.

### Example 6

### Salivary MIRNA and OPC Patient Survival

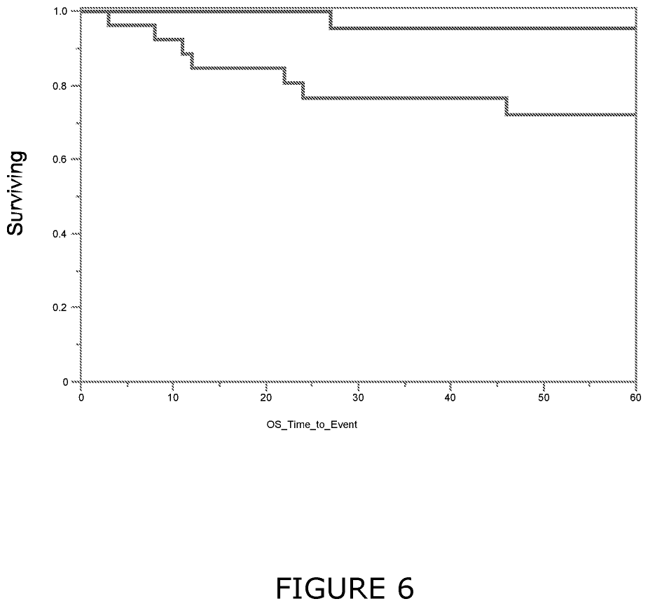

The prognostic potential of these miRNAs was evaluated using Cox proportional hazard modeling. Survival data (up to 5 years) were available for 50 OPC patients and survival characteristics were evaluated in terms of overall survival (OS). Among the miRNA investigated, hsa-miR-07-5p (HR=0.638, P=0.001) was identified to have a significant association with OPC prognosis whereas down-regulation was associated with poor prognosis. Kaplan Meier analysis considering the median split confirmed the association (Log-rank test; X² (1, N=50)=4.555, P=0.033, Wilcoxon test X² (1, N=50)=4.670, P=0.031) (FIG. 6).

### Discussion of Examples 1-6

Considering the rising incidence of HPV-associated OPC and the lack of early detection methods, salivary miRNA changes in HPV-associated OPC were investigated to identify candidate miRNAs with diagnostic potential. Traditionally, liquid biopsy-based cancer biomarker studies underpin biomarker discovery on changes in the tumor tissue and subsequent detection of such changes in body fluids. Conversely, a distinct discovery approach was employed by the present inventors, in which salivary samples of HPV-associated OPC patients and controls were directly analyzed using next-generation sequencing to discover dysregulated miRNA in saliva. This approach allowed collective identification of detectable salivary miRNA expression changes that are associated with either miRNA changes in the tumor, tumor microenvironment or in the surrounding normal tissue in response to the developing tumor.

The present inventors also investigated miRNA expression changes that can discriminate HPV-associated OPC patients from HPV-positive controls. Viral infections, such as HPV, itself trigger active and passive changes of miRNA expression (Cullen et al., 2013, supra). Even though infection associated miRNA changes can play a role in virus-driven cancer detection, these markers hold limited value in HPV driven cancers as the vast majority of infections spontaneously resolve within one to two years leaving only a small percentage (approximately 1-2%) to ever develop HPV driven cancers (Kang et al., Cancer Sci. 2020; 111(10):3854-3861; Kreimer et al., Cancer. 2018; 124(9):1859-66). By comparing salivary miRNA expression patterns between HPV-positive OPC patients and HPV-positive controls, this present disclosure reveals that certain salivary miRNA expression changes are capable of effectively discriminating those who have developed HPV-associated cancers from the HPV-positive controls. In the discovery stage, 17 miRNA were identified to be differently expressed between these groups. 11 miRNAs were considered for the validation and among them, seven were confirmed to be differentially expressed between these groups. Furthermore, a panel consisting of three miRNAs (Hsa-miR-194-5p, Hsa-miR-501-3p, Hsa-miR-548K) were identified to have an AUC of 90.6% (83.3%, 97.9%) for identification of HPV-positive OPC patients from HPV-positive controls.

Even though the main objective of the present disclosure was to investigate miRNA changes associated with HPV-positive OPC, several HPV-negative OPC salivary samples were also evaluated for the 14 miRNA targets that were considered in the validation stage. Among them, 8/14 miRNAs were observed to be differentially expressed between HPV-negative OPC and HPV-negative controls. A panel consisting of seven miRNA (Hsa-miR-215-3p, Hsa-miR-194-5p, Hsa-miR-449a, Hsa-miR-07-5p, Hsa-miR-501-3p, Hsa-miR-1290 and Hsa-miR-548K) could differentiate HPV-negative OPC from HPV-negative controls with an AUC of 95.5% (90.6%, 100.0%). Considering the presence of common OPC-driven miRNA changes the efficacy of these miRNA was further evaluated to differentiate OPC from controls regardless of their HPV status. Statistical modeling identified a panel consisting of 4 miRNAs (Hsa-miR-3614-5p, Hsa-miR-501-3p, Hsa-miR-1290, Hsa-miR-1246) which can distinguish OPC from controls with an AUC of 77.9% (69.6%, 86.1%).

The current disclosure also points out that salivary miRNAs have the potential to predict OPC patient outcomes. Among the 14 miRNA targets considered in the validation, Hsa-miR-07-5p had a significant association with OPC prognosis where lower salivary expression of Hsa-miR-07-5p indicated a poor overall survival.

While providing insight into salivary miRNA expression changes associated with HPV-positive and negative OPC, it is proposed that these changes can be used to detect OPC in early-stage and predict patient outcomes in advance. More importantly, the present disclosure reveals that there are detectable salivary miRNA changes in HPV-positive OPC patients compared to HPV infected individuals uncovering a novel approach for early detection of HPV driven OPC by coupling salivary HPV detection and miRNA evaluation. Due to its non-invasive nature and being one of the most convenient specimens to collect, saliva is an ideal matrix for screening purposes. As such, the proposed biomarkers are considered to play a pivotal role in the early detection and management of OPC.

### Example 7

### Secondary Validation of Salivary MIRNA Biomarkers Using an Independent Cohort of Patients and an Independent Analysis Platform

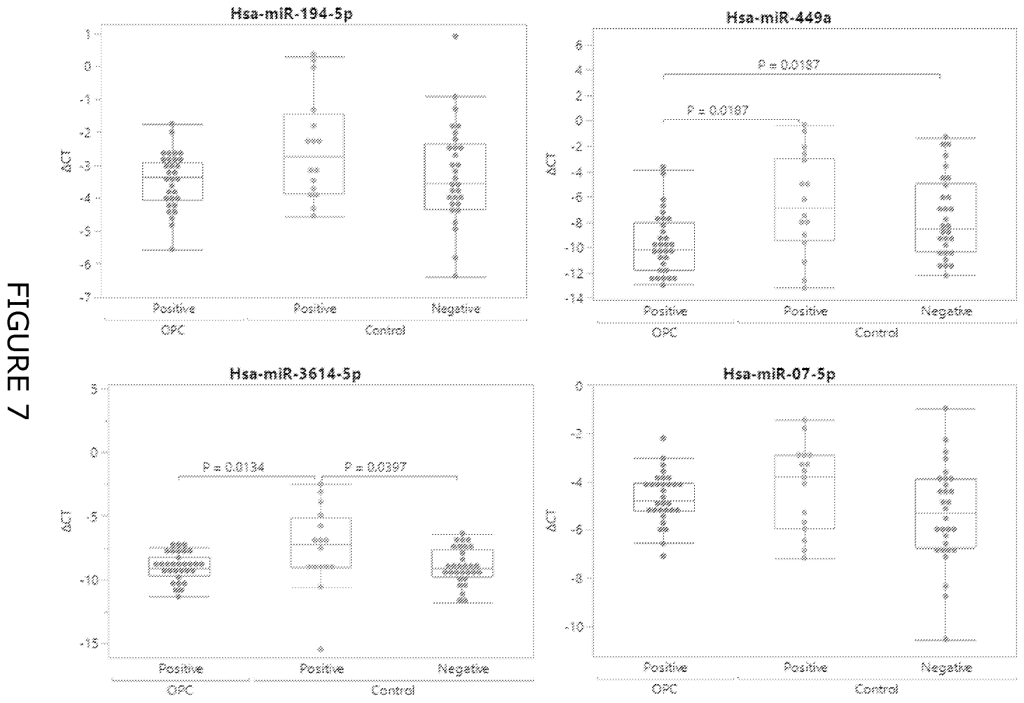

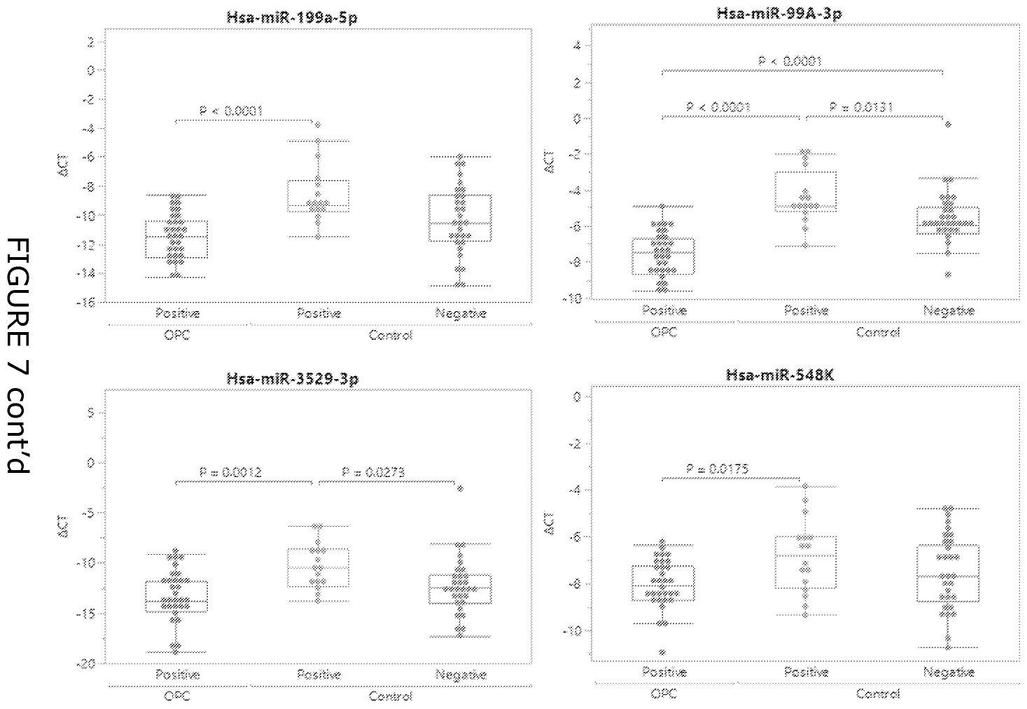

A secondary validation was performed using novel miRCURY™ LNA primer PCR assays (QIAGEN, MD, USA) in an additional cohort of HPV-positive OPC patients (N=31), HPV-negative controls (N=30) (Table 6). Due to the unavailability of additional HPV-positive controls, the same cohort was re-tested using the LNA based platform (Table 7, FIG. 7).

TABLE 6
Demographic and clinical characteristics of participants considered
for qPCR validation using miRCURY ™ LNA primer PCR assays.

| | | HPV-positive | HPV-positive | HPV-negative |
|---|---|---|---|---|
| | | OPC | controls | controls |
| | | (N = 31) | (N = 16) | (N = 30) |
| Age | <=55 | 9 (30.0%) | 8 (50.0%) | 6 (20.0%) |
| | 56-65 | 13 (43.3%) | 4 (25.0%) | 7 (23.3%) |
| | >=66 | 8 (26.7%) | 4 (25.0%) | 17 (56.7%) |
| | Mean | 61.23 (9.77) | 56.56 (12.73) | 66.33 (10.33) |
| | (SD) | | | |
| Gender | Male | 25 (80.6%) | 13 (81.2%) | 28 (93.3%) |
| | Female | 2 (6.5%) | 3 (18.8%) | 2 (6.7%) |
| OPC site | Tonsil | 21 (67.7%) | — | — |
| | BOT | 10 (32.3%) | | |
| AJCC | Stage 01 | 21 (67.7%) | — | — |
| stage (8^{th} | Stage 02 | 9 (29.0%) | | |
| Edition) | Stage 03 | 1 (3.2%) | | |

TABLE 7
One-way Kruskal-Wallis analysis with Steel-Dwass pairwise comparison of salivary
miRNA expression - Revalidation using miRCURY ™ LNA primer PCR assays.

| | | | | Steel Dwass multiple comparison procedure P | | |
|---|---|---|---|---|---|---|
| | | | | VALUES - valid only if Kruskal Wallis is significant | | |
| | | | | HPV Positive | HPV Positive | HPV Positive |
| | Kruskal Wallis Test | | | OPC vs HPV | Control vs HPV | OPC vs HPV |
| | | | Prob > | Positive | Negative | Negative |
| miRNA | Chi-square | DF | ChiSq | Control | Control | Control |
| Hsa-miR-194-5p | 3.8816 | 2 | 0.1436 | 0.1331 | 0.2751 | 0.9050 |
| Hsa-miR-449a | 10.8914 | 2 | 0.0043* | 0.0187* | 0.6040 | 0.0187* |
| Hsa-miR-199a-5p | 17.2772 | 2 | 0.0002* | <.0001* | 0.0534 | 0.1236 |
| Hsa-miR-3614-5p | 8.6950 | 2 | 0.0129* | 0.0134* | 0.0397* | 0.8924 |
| Hsa-miR-07-5p | 4.9813 | 2 | 0.0829 | 0.4392 | 0.1252 | 0.2942 |
| Hsa-miR-3529-3p | 13.0137 | 2 | 0.0015* | 0.0012* | 0.0273* | 0.4250 |
| Hsa-miR-99A-3p | 40.0824 | 2 | <.0001* | <.0001* | 0.0131* | <.0001* |
| Hsa-miR-501-3p | 21.3522 | 2 | <.0001* | 0.0001* | 0.5025 | 0.0009* |
| Hsa-miR-1290 | 20.4131 | 2 | <.0001* | 0.0004* | 0.0185* | 0.0062* |
| Hsa-miR-548K | 7.6204 | 2 | 0.0221* | 0.0175* | 0.3934 | 0.2608 |
| Hsa-miR-1246 | 19.8510 | 2 | <.0001* | <.0001* | 0.0131* | 0.0664 |

### Example 9

Salivary MIRNA Expression—HPV positive OPC Vs HPV Positive Controls

A comparison between HPV-positive OPC and HPV-positive controls identified seven miRNAs with significant differences in salivary expression. These miRNAs were downregulated in salivary samples of HPV-positive OPC patients compared to non-cancer patients with oral HPV infection. (FIG. 1, Table 7). Among them, Hsa-miR-548K was the most significantly dysregulated miRNA between these groups (P=0.0006).

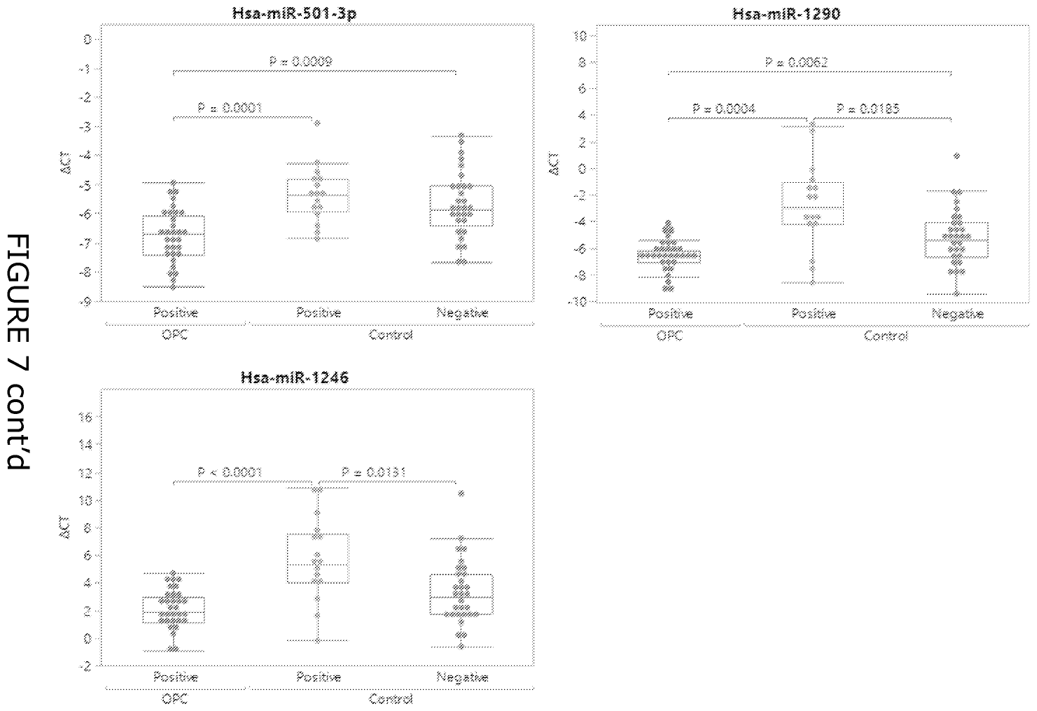

Regression modelling identified a salivary diagnostic biomarker panel consisting of 9 miRNAs (Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290, Hsa-miR-548K) with the potential to differentiate HPV-positive OPC patients from HPV-positive controls (FIG. 8 A-B). The panel had a sensitivity of 91.3% (79.7%, 96.6%) and a specificity of 86.7% (62.1%, 96.3%). Receiver operating characteristic (ROC) analysis revealed that the panel has an AUC value of 94.8% (89.6%, 100%). Secondary validation revealed this panel has an AUC of 98.0% (94.3%, 100%) to distinguish HPV-positive OPC patients from HPV-positive controls (FIG. 8 C-D).

### Example 10

### Salivary MIRNA Expression—HPV Negative OPC Vs HPV Negative Controls

Although salivary miRNA changes in HPV-negative OPC patients were not investigated at the discovery phase, several miRNAs considered in the validation phase showed differential expression in the saliva of HPV-negative OPC patients compared to HPV-negative controls (FIG. 2, Table 5). Eight of these miRNA were observed to be significantly dysregulated between these groups including Hsa-miR-07-5p (P=0.0002), displaying the most significant differential expression.

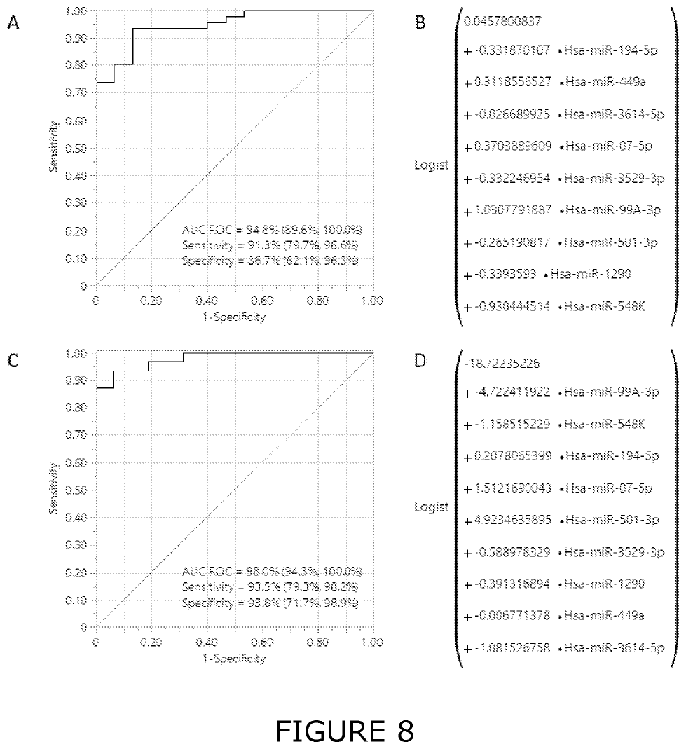

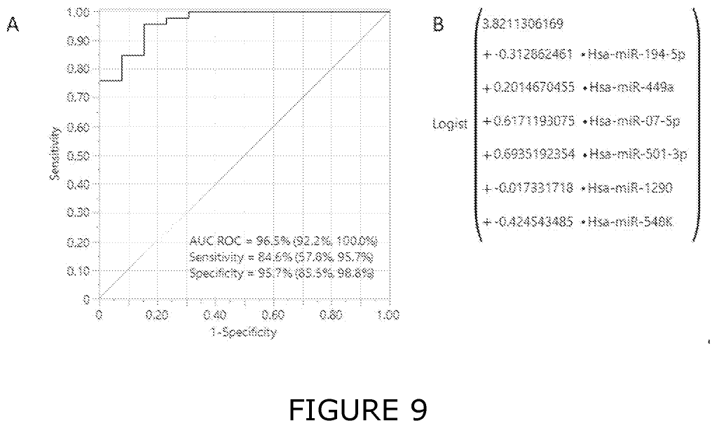

Regression analysis revealed that a salivary biomarker panel consisting of six miRNAs (Hsa-miR-194-5p, Hsa-miR-449a, Hsa-miR-07-5p, Hsa-miR-501-3p, Hsa-miR-1290, Hsa-miR-548K) could distinguish HPV-negative OPC patients from controls with a sensitivity of 84.6% (57.8%, 95.7%) and a specificity of 95.7% (85.5%, 98.8%) (FIG. 9 A-B). The AUC value of the panel was 96.5% (92.2%, 100.0%) (FIG. 9 A-B). As additional samples of HPV-negative OPCs were not available a secondary validation could not be performed. However, cross-validation revealed an AUC of 79.5% (67.7%, 91.3%) for the panel to distinguish HPV-negative OPC patients from HPV-negative controls.

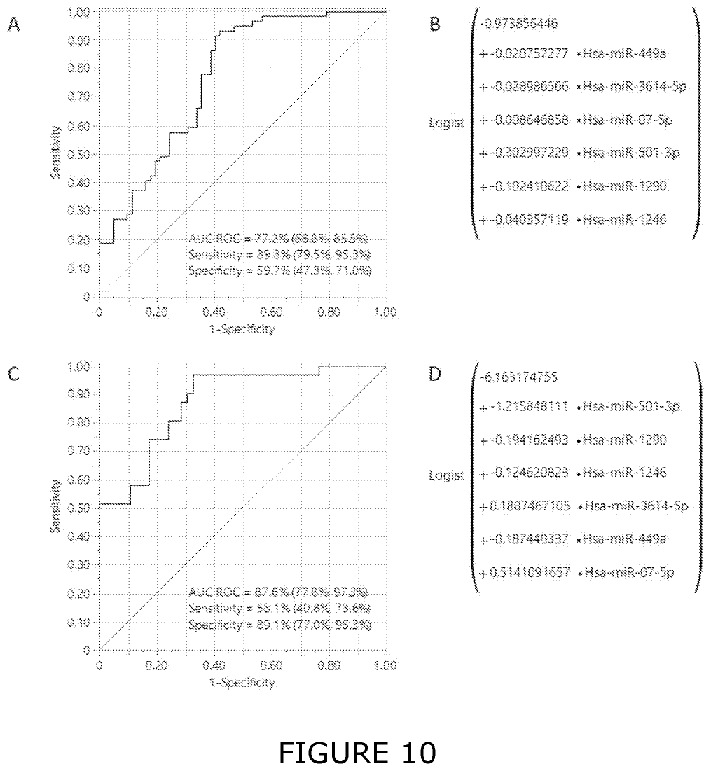

Considering that several miRNAs are associated with both HPV-positive and HPV-negative OPC, overall results were analyzed to identify salivary miRNA candidates capable of predicting OPC irrespective of the HPV status. The analysis identified a panel consisting of six miRNAs (Hsa-miR-449a, Hsa-miR-3614-5p, Hsa-miR-07-5p, Hsa-miR-501-3p, Hsa-miR-1290, Hsa-miR-548K) that is capable of differentiating OPC patients from controls with a sensitivity of 89.8% (79.5%, 95.3%) and a specificity of 59.7% (47.3%, 71.0%). The AUC of the panel was 77.2% (68.8%, 85.5%) (FIG. 10 A-B). Secondary validation revealed a sensitivity of 58.1% (40.8%, 73.6%), specificity of 89.1% (77.0%, 95.3%) and an AUC of 87.6% (77.8%, 97.3%) for the assay to distinguish OPC patients from controls (FIG. 10 C-D).

### Materials and Methods

### Ethical Considerations

Ethical clearance was obtained from the Metro South Human Research Ethics Committee [HREC/12/QPAH/381]. The study was also approved by Queensland University of Technology [HREC No: 1400000617, 1400000641 and 200000043], University of Queensland Medical Ethical Institutional Board [HREC No: 2014000862] and Royal Brisbane and Women's Hospital (RBWH) [HREC/16/QRBW/447].

### Patient Recruitment

Sample collection was carried out from 2012 to 2020 in Queensland, Australia. Treatment naive OPC patients were recruited from Princess Alexandra Hospital, Royal Brisbane and Women's Hospital, and Logan Hospital. Cancer-free individuals were recruited from The University of Queensland School of Dentistry (UQDS), The Queensland University of Technology Health Clinics (QUTHC), Logan Hospital, and Metro-North Sexual Health and HIV Service. OPC patients were clinically followed-up up to 5 years. Survival characteristics were evaluated in terms of overall survival (OS).

### Cyclin-Dependent Kinase Inhibitor 2A (P16) Immunohistochemistry

OPC tumor tissues/biopsies were tested for p16 immunohistochemistry by Queensland pathologists as a part of the routine clinical assessment (Ekanayake Weeramange et al., The Journal of Molecular Diagnostics. 2021; 23(10):1334-42). CINtec@p16INK4a Histology Kit (E6H4 clone) (Roche MTM Laboratories, Heidelberg, Germany) was used for the assay and strong diffuse nuclear and cytoplasmic staining present over 70% of tumor tissue was considered as positive for p16.

### Saliva Collection and Processing

Samples were collected according to a previously described method (Tang et al., Head & neck. 2019; 41(5):1484-9; Sun et al., Diagnostics (Basel). 2017; 7(1)). Briefly, participants were requested to refrain from eating or drinking for at least 1 hour prior to saliva collection. Following passive pooling, 2-5 mL of saliva was collected by expectorating into a collection container. Samples were transported on ice and immediately processed before storing at −80° C. Saliva for miRNA isolation was processed by mixing 200 μL of saliva with 800 μL of QIAzol (QIAGEN, MD, USA). The remaining saliva was aliquoted and stored as neat saliva.

### HPV Detection

DNA was isolated from neat saliva using QIAamp DNA Mini Kit (QIAGEN, MD, USA) according to a previously described procedure (Tang et al., 2019, supra). Salivary DNA was tested for HPV16 by quantitative PCR and HPV16-negative samples were tested for 17 high-risk types by iPlex MassARRAY (Agena Bioscience, CA, USA). Detailed procedure is described elsewhere (Ekanayake Weeramang et al., 2021, supra).

### MIRNA Isolation

miRNA isolation was carried out using NucleoSpin™ miRNA isolation kit (Machnery-Nagel, Düren, Germany) according to a previously published protocol with a few minor modifications (Salazar et al., Cellular Oncology. 2014; 37(5):331-8; Wan et al., Oncotarget. 2017; 8(59):99990-100001). The fraction containing 200 μL of saliva with 800 μL of QIAzol (QIAGEN, MD, USA) was used. The mixture was vortexed and incubated at room temperature for 5 minutes. 140 μL of Chloroform was added and the mixture was further incubated for 3 minutes at room temperature. Following the centrifugation at 12000×g for 15 minutes at 4° C., the clear supernatant was transferred to a separate vial. 200 μL of ethanol was added to the separated supernatant and large RNAs were isolated by column filtration. 800 μL of buffer MX was added to the filtrate obtained by the previous step and small RNAs were isolated separately using column filtration. Following 3 consecutive washing steps with 600 μL of MW1, 700 μL of MW2, and 250 μL of MW1, small RNAs were eluted using 20 μL of ultra-pure water. Qubit microRNA assay (Thermo Fisher Scientific, USA) was used for the quantification of isolated miRNA.

### Small RNA Sequencing

Library preparation and small RNA sequencing were carried out at BGI Genomics (New Territories, Hong Kong). DNBSEQ™ technology involving combinatorial probe-anchor synthesis (cPAS), linear isothermal rolling-circle replication, and DNA nanoballs (DNB™) technology was used for sequencing. Quantification accuracy was improved by using unique molecular identifiers (UMIs).

### MIRNA Quantification by Quantitative Reverse Transcription PCR (RT-QPCR)

Complementary DNA (cDNA) synthesis was carried out using miScript II™ RT Kit (QIAGEN, MD, USA) as per the manufacturer's protocol. 300 ng of isolated small RNA was used as the input. Briefly, 4 μL of 5× miScript HiSpec™ Buffer, 2 μL of 10× miScript Nucleics™ Mix & 2 μL of miScript Reverse Transcriptase Mix was added to the template miRNA and total volume was adjusted to 20 μL using RNase-free water. The mixture was incubated for 60 minutes at 37° C. and further 5 minutes at 95° C. to heat inactivate the enzymes. The cDNA synthesis process involves polyadenylation of mature miRNAs by poly(A) polymerase and reverse transcription of miRNA using oligo-dT primers containing a universal tag sequence.

Custom miScript™ primer PCR Assays (QIAGEN, MD, USA) were used for qPCR amplification of the selected miRNAs. The assay employs a target-specific forward primer and a universal reverse primer directed towards the universal tag incorporated in the cDNA synthesis step. 5 μL of 2× QuantiTect™ SYBR Green PCR master mix, 1 μL of 10× miScript universal primer, 1 μL of 10× miScript primer assay (target-specific primer), and 6 ng of cDNA adjusted to 3 μL using RNase-free water were used per each reaction. Amplification was performed using QuantStudio 7 Flex Real-Time PCR System (Applied Biosystems). PCR conditions were 95° C. for 15 minutes for the initial activation of HotStart™ Taq DNA polymerase, 40 cycles at 94° C. for 15 seconds, 55° C. for 30 seconds, and 70° C. for 30 seconds followed by a melt curve analysis at 95° C. for 15 seconds, 60° C. for 1 minute and 95° C. for 15 seconds. All samples were tested in duplicate.

### MiRCURY™ Locked Nucleic Acid (LNA) MIRNA PCR Assays

miRCURY® LNA® RT Kit was used for cDNA synthesis and 48 ng of small RNA was used in each reaction. Two microliters of 5× miRCURY RT reaction buffer, 1 μL of 10× miRCURY RT enzyme mix, 48 ng of small RNA adjusted to 7 μL using RNA free water was used per each reverse transcription reaction. Reaction mixture was incubated for 60 minutes at 42° C. and subsequently 5 minutes at 95° C. for heat inactivation of reverse transcriptase enzyme.

Custom miRCURY™ Locked Nucleic Acid (LNA) miRNA PCR Assays (QIAGEN, MD, USA) were used for qPCR amplification of the selected miRNAs. The assay employs LNA based target-specific forward primer and reverse primer for efficient miRNA quantification. 5 μL of 2× miRCURY SYBR Green PCR master mix, 0.5 μL of ROX reference dye, 1 μL of PCR primer mix, and 1.5 ng of cDNA adjusted to 3.5 μL using RNase-free water were used per each reaction. PCR amplification was performed using QuantStudio 6 Flex Real-Time PCR System (Applied Biosystems). PCR conditions were 95° C. for 2 minutes for the heat activation of HotStart Taq DNA polymerase, 40 cycles at 95° C. for 10 seconds, 56° C. for 60 seconds, followed by a melt curve analysis at 95° C. for 15 seconds, 60° C. for 1 minute and 95° C. for 15 seconds. All samples were tested in duplicate.

### Statistical Methods

### Bioinformatics Analysis of Sequencing Data

The overall quality of raw single-end reads was assessed using FastQC (Andrews S. FastQC [https://www.bioinformatics.babraham.ac.uk/projects/fastqc/]. 0.11.9 ed2010). Adaptor sequences and poor-quality reads were then removed using Trim Galore (Krueger F. Trim Galore [https://www.bioinformatics.babraham.ac.uk/projects/trim galore/]. 0.6.5 ed2012). Human miRNA sequences were downloaded from miRbase release 22.1 (Kozomara et al., Nucleic Acids Res. 2019; 47(D1):D155-D62). High-quality reads were mapped onto reference human mature and complementary miRNA sequences using bowtie (Langmead et al., Genome Biol. 2009; 10(3):R25) allowing up to a single mismatch. Feature counts for individual miRNA sequences were determined using SAMtools idxstats (Li et al., Bioinformatics. 2009; 25(16):2078-9). Differentially expressed miRNAs were determined using DESeq2 (Love et al., Genome Biol. 2014; 15(12):550).

qPCR Data Analysis

The relative expression (ΔCt) was calculated considering SNORD-96A as the reference miRNA (Salazar et al., 2014, supra; Wan et al., 2017, supra; Manfe et al., PLoS ONE. 2013; 8(3):e59390). MiRNA expression patterns between groups were investigated using Kruskal-Wallis analysis. Steel Dwass multiple comparison method was employed for investigating pair-wise associations. Performance characteristics of miRNA were evaluated using a generalized regression model using LASSO penalized regression for model selection and Leave-One-Out cross-validation. The cut-offs for sensitivity and specificity were chosen to balance sensitivity and specificity with a slight preference for better sensitivity in the training set. Survival characteristics were evaluated using Cox Proportional Hazards modelling with LASSO model selection as before. Statistical analysis was conducted using JMP Pro software version 17.0.0 (SAS Institute, Cary, NC, USA). Survival characteristics were evaluated using Cox Proportional Hazards modeling.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

What is claimed is:

1. A method for determining an indicator used in assessing a likelihood that human papillomavirus (HPV)-positive oropharyngeal cancer (OPC) or OPC-negative HPV infection is present or absent in a human subject, the method comprising, consisting or consisting essentially of:
(1) determining a biomarker value for at least one miRNA biomarker (e.g., 1, 2 or 3 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-501-3p and Hsa-miR-548K; and
(2) determining the indicator using the biomarker value(s).

2. A method for determining an indicator used in assessing a likelihood that HPV-positive OPC or OPC-negative HPV infection is present or absent in a human subject, the method comprising, consisting or consisting essentially of:
(1) determining a biomarker value for at least one miRNA biomarker (e.g., 1, 2, 3, 4, 5, 6, 7, 8 or 9 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K; and
(2) determining the indicator using the biomarker value(s).

3. A method for determining an indicator used in assessing a likelihood that HPV-negative OPC or an OPC-negative, HPV-negative condition is present or absent in a human subject, the method comprising, consisting or consisting essentially of:
(1) determining a biomarker value for at least one miRNA biomarker (e.g., 1, 2, 3, 4, 5, 6 or 7 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K; and
(2) determining the indicator using the biomarker value(s).

4. A method for determining an indicator used in assessing a likelihood that HPV-negative OPC or an OPC-negative, HPV-negative condition is present or absent in a human subject, the method comprising, consisting or consisting essentially of:
(1) determining a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-194-5p, Hsa-miR-449a, Hsa-miR-07-5p, Hsa-miR-501-3p, Hsa-miR-1290 and Hsa-miR-548K; and
(2) determining the indicator using the biomarker value(s).

5. A method for determining an indicator used in assessing a likelihood that HPV-agnostic OPC or an OPC-negative condition is present or absent in a human subject, the method comprising, consisting or consisting essentially of:
(1) determining a biomarker value for at least one miRNA biomarker (e.g., 1, 2, 3 or 4 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-3614-5p, Hsa-miR-501-3p, Hsa-miR-1290 and Hsa-miR-1246; and
(2) determining the indicator using the biomarker value(s).

6. A method for determining an indicator used in assessing a likelihood that HPV-agnostic OPC or an OPC-negative condition (e.g., HPV-positive, OPC-negative condition, HPV-negative, OPC-negative condition) is present or absent in a human subject, the method comprising, consisting or consisting essentially of:
(1) determining a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, and wherein the at least one miRNA biomarker is selected from Hsa-miR-449a, Hsa-miR-3614-5p, Hsa-miR-07-5p, Hsa-miR-501-3p, Hsa-miR-1290 and Hsa-miR-548K; and
(2) determining the indicator using the biomarker value(s).

7. The method of any one of claims 1 to 6, wherein the subject has at least one clinical sign of OPC, which is suitably selected from sore throat that does not go away, a lump in the throat, mouth or neck, trouble swallowing, trouble opening the mouth fully, trouble moving the tongue, weight loss for no reason, ear pain, a change in voice, a white patch on the tongue or lining of the mouth that does not go away, and coughing up blood).

8. The method of any one of claims 1 to 7, further comprising combining the biomarker values to provide a composite score and determining the indicator using the composite score.

9. The method of any one of claims 1 to 8, further comprising analyzing the biomarker value(s) or composite score with reference to one or more reference biomarker values, value ranges or cut-off values, or reference composite scores, composite score ranges or composite score cut-offs, to determine the indicator.

10. The method of claim 9, wherein the indicator indicates a likelihood of a presence of a condition selected from HPV-positive OPC, OPC-negative HPV infection, HPV-negative OPC, HPV-agnostic OPC, OPC-negative, HPV-negative condition, OPC-negative condition and OPC-negative, HPV-negative condition, if the biomarker value(s) or composite score is indicative of the level of the biomarker(s) in the sample that correlates with an increased likelihood of a presence of the condition relative to a predetermined reference biomarker value, value range or cut-off value, or to a predetermined reference composite score, composite score range or composite score cut-off.

11. A method for determining an indicator used in assessing a likelihood of a human subject with OPC having a survival prognosis selected from a decreased or poor survival prognosis and an increased or good survival prognosis, the method comprising, consisting or consisting essentially of:
(1) determining a biomarker value for Hsa-miR-07-5p in a saliva sample obtained from the subject, wherein the biomarker value is indicative of a level of Hsa-miR-07-5p in the sample; and
(2) determining the indicator using the biomarker value.

12. The method of claim 11, further comprising analyzing the biomarker value with reference to a reference biomarker value, value range or cut-off value, to determine the indicator.

13. The method of claim 12, wherein the indicator indicates a likelihood of a decreased or poor survival prognosis if the biomarker value for Hsa-miR-07-5p is indicative of a level of Hsa-miR-07-5p in the sample that correlates with an increased likelihood of a decreased or poor survival prognosis relative to a predetermined reference biomarker value, value range or cut-off value.

14. The method of claim 12, wherein the indicator indicates a likelihood of an increased or good survival prognosis if the biomarker value for Hsa-miR-07-5p is indicative of a level of Hsa-miR-07-5p in the sample that correlates with an increased likelihood of an increased or good survival prognosis relative to a predetermined reference biomarker value, value range or cut-off value.

15. An apparatus for determining an indicator used in assessing a likelihood that HPV-positive OPC or OPC-negative HPV infection is present or absent in a human subject, the apparatus comprising at least one electronic processing device that:
determines a biomarker value for at least one miRNA biomarker (e.g., 1, 2 or 3 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-501-3p and Hsa-miR-548K; and
determines the indicator using the derived biomarker value(s).

16. An apparatus for determining an indicator used in assessing a likelihood that HPV-positive OPC or an OPC-negative, HPV-positive condition is present or absent in a human subject, the apparatus comprising at least one electronic processing device that:
determines a biomarker value for at least one miRNA biomarker (e.g., 1, 2, 3, 4, 5, 6, 7, 8 or 9 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-449a, Hsa-miR-3614-5p, Hsa-miR-07-5p, Hsa-miR-3529-3p, Hsa-miR-99A-3p, Hsa-miR-501-3p, Hsa-miR-1290 and Hsa-miR-548K; and
determines the indicator using the derived biomarker value(s).

17. An apparatus for determining an indicator used in assessing a likelihood that HPV-negative OPC or an OPC-negative, HPV-negative condition is present or absent in a human subject, the apparatus comprising at least one electronic processing device that:
determines a biomarker value for at least one miRNA biomarker (e.g., 1, 2, 3, 4, 5, 6 or 7 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-215-3p, Hsa-miR-194-5p, Hsa-miR-449a, Hsa-miR-07-5p, Hsa-miR-501-3p, Hsa-miR-1290 and Hsa-miR-548K; and
determines the indicator using the derived biomarker value(s).

18. An apparatus for determining an indicator used in assessing a likelihood that HPV-negative OPC or an OPC-negative, HPV-negative condition is present or absent in a human subject, the apparatus comprising at least one electronic processing device that:
determines a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-194-5p, Hsa-miR-449a, Hsa-miR-07-5p, Hsa-miR-501-3p, Hsa-miR-1290 and Hsa-miR-548K; and
determines the indicator using the derived biomarker value(s).

19. An apparatus for determining an indicator used in assessing a likelihood that HPV-agnostic OPC or an OPC-negative condition is present or absent in a human subject, the apparatus comprising at least one electronic processing device that:
determines a biomarker value for at least one miRNA biomarker (e.g., 1, 2, 3 or 4 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-3614-5p, Hsa-miR-501-3p, Hsa-miR-1290 and Hsa-miR-1246; and
determines the indicator using the derived biomarker value(s).

20. An apparatus for determining an indicator used in assessing a likelihood that HPV-agnostic OPC or an OPC-negative condition (e.g., HPV-positive, OPC-negative condition, HPV-negative, OPC-negative condition) is present or absent in a human subject, the apparatus comprising at least one electronic processing device that:
determines a biomarker value for at least one miRNA biomarker (e.g., 1, 2, 3, 4, 5 or 6 miRNA biomarkers) in a saliva sample obtained from the subject, wherein a respective biomarker value is indicative of a level of a corresponding miRNA biomarker in the sample, wherein the at least one miRNA biomarker is selected from Hsa-miR-449a, Hsa-miR-3614-5p, Hsa-miR-07-5p, Hsa-miR-501-3p, Hsa-miR-1290 and Hsa-miR-548K; and
determines the indicator using the derived biomarker value(s).

21. An apparatus for determining an indicator used in assessing a likelihood of a human subject with OPC having a survival prognosis selected from a decreased or poor survival prognosis and an increased or good survival prognosis, the apparatus comprising at least one electronic processing device that:
determines a biomarker value for Hsa-miR-07-5p in a saliva sample obtained from the subject, wherein the biomarker value is indicative of a level of Hsa-miR-07-5p in the sample; and
determines the indicator using the derived biomarker value.

22. A composition comprising a mixture of a DNA polymerase, saliva cDNA from a human subject, suitably with at least one clinical sign of OPC, wherein the saliva cDNA comprises at least one cDNA biomarker (e.g., 1, 2 or 3 cDNA biomarkers) selected from Hsa-miR-194-5p cDNA, Hsa-miR-501-3p cDNA and Hsa-miR-548K cDNA, and wherein the composition further comprises for a respective cDNA biomarker at least one oligonucleotide primer or probe that hybridizes to the cDNA biomarker.

23. A composition comprising a mixture of a DNA polymerase, saliva cDNA from a human subject, suitably with at least one clinical sign of OPC, wherein the saliva cDNA comprises at least one cDNA biomarker (e.g., 1, 2, 3, 4, 5, 6, 7, 8 or 9 cDNA biomarkers) selected from Hsa-miR-194-5p cDNA, Hsa-miR-449a cDNA, Hsa-miR-3614-5p cDNA, Hsa-miR-07-5p cDNA, Hsa-miR-3529-3p cDNA, Hsa-miR-99A-3p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

24. A composition comprising a mixture of a DNA polymerase, saliva cDNA from a human subject, suitably with at least one clinical sign of OPC, wherein the saliva cDNA comprises at least one cDNA biomarker (e.g., 1, 2, 3, 4, 5, 6 or 7 cDNA biomarkers) selected from Hsa-miR-215-3p cDNA, Hsa-miR-194-5p cDNA, Hsa-miR-449a cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

25. A composition comprising a mixture of a DNA polymerase, saliva cDNA from a human subject, suitably with at least one clinical sign of OPC, wherein the saliva cDNA comprises at least one cDNA biomarker (e.g., 1, 2, 3, 4, 5 or 6 cDNA biomarkers) selected from Hsa-miR-194-5p cDNA, Hsa-miR-449a cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

26. A composition comprising a mixture of a DNA polymerase, saliva cDNA from a human subject, suitably with at least one clinical sign of OPC, wherein the saliva cDNA comprises at least one cDNA biomarker (e.g., 1, 2, 3 or 4 miRNA biomarkers) selected from Hsa-miR-3614-5p cDNA, Hsa-miR-501-3p cDNA, Hsa-miR-1290 cDNA and Hsa-miR-1246 cDNA, and wherein the composition further comprises for a respective cDNA biomarker at least one oligonucleotide primer or probe that hybridizes to the cDNA biomarker.

27. A composition comprising a mixture of a DNA polymerase, saliva cDNA from a human subject, suitably with at least one clinical sign of OPC, wherein the saliva cDNA comprises at least one cDNA biomarker (e.g., 1, 2, 3, 4, 5 or 6 cDNA biomarkers) selected from Hsa-miR-449a cDNA, Hsa-miR-3614-5p cDNA, Hsa-miR-07-5p cDNA, Hsa-miR-501-3p cDNA, Hsa-miR-1290 cDNA and Hsa-miR-548K cDNA, and wherein the composition further comprises for a respective cDNA biomarker at least one oligonucleotide primer or probe that hybridizes to the cDNA biomarker.

28. A composition comprising a mixture of a DNA polymerase, saliva cDNA from a human subject, suitably with at least one clinical sign of OPC, wherein the saliva cDNA comprises a cDNA biomarker corresponding to Hsa-miR-07-5p, and at least one oligonucleotide primer or probe that hybridizes to the cDNA biomarker.

29. A device for nucleic acid amplification of saliva cDNA, the device comprising a plurality of reaction vessels, individual reaction vessels comprising the composition of any one of claims 22 to 28.

30. A method for inhibiting the development or progression of OPC in a human subject, the method comprising: exposing the subject to a treatment regimen for OPC at least in part on the basis that the subject is determined by the indicator-determining method of any of claims 1 to 15 as having a likelihood of a presence of an OPC condition selected from HPV-positive OPC, HPV-negative OPC and HPV-agnostic OPC.
